(19) United States
(12) Patent Application Publication
(10) Pub. No.: US 2003/0157084 A1
(43) Pub. Date: Aug. 21, 2003

(54) Methods for preparing purified prostaglandin E synthase

(76) Inventors: Per-Johan Jakobsson, Stockholm (SE); Rolf Weinander, Karlstad (SE); Ralf Morgenstern, Stockholm (SE); Ralph H. Lambalot, Wrentham, MA (US)

Correspondence Address: FOLEY HOAG, LLP, PATENT GROUP, WORLD TRADE CENTER WEST, 155 SEAPORT BLVD, BOSTON, MA, 02110, US

(21) Appl. No.: 10/227,617

(22) Filed: Aug. 23, 2002

Related U.S. Application Data
(60) Provisional application No. 60/314,853, filed on Aug. 24, 2001.
(60) Provisional application No. 60/315,405, filed on Aug. 28, 2001.

(51) Int. Cl. A61K 38/44; C12N 9/02

(52) U.S. Cl. 424/94.4; 435/189

(57) ABSTRACT

The invention provides purified preparations of prostaglandin E synthase (PGES), analogs and variants thereof, such as portions thereof, and fusion polypeptides comprising these. The invention also provides methods for obtaining these purified preparations; methods for identifying modulators of PGES, e.g., small molecules inhibiting or stimulating the activity of PGES; and methods for preparing prostaglandins. Nucleic acids encoding PGES polypeptides of the invention are also provided.

### CROSS-REFERENCE TO RELATED APPLICATIONS

This application claims the benefit of U.S. Provisional Application No. 60/314,853, filed Aug. 24, 2001 and U.S. Provisional Application No. 60/315,405, filed Aug. 28, 2001, the contents of which are specifically incorporated by reference herein.

### FIELD OF THE INVENTION

The present invention relates to pure preparations of prostaglandin E synthase (PGES), analogs and variants thereof, and novel methods for preparing purified PGES. The invention also provides screening methods for identifying modulators of PGES and methods for preparing prostaglandins and analogs thereof.

### BACKGROUND OF THE INVENTION

Prostaglandins (which include PGE₂, PGD₂, PGF_{2α}, PGI₂ and other related compounds) represent a diverse group of autocrine and paracrine hormones that are derived from the metabolism of fatty acids. They belong to a family of naturally occurring eicosanoids (prostaglandins, thromboxanes and leukotrienes) which are not stored as such in cells, but are biosynthesized on demand from arachidonic acid, a 20-carbon fatty acid that is derived from the breakdown of cell-membrane phospholipids. Under normal circumstances, the eicosanoids are produced at low levels to serve as important mediators of many and diverse cellular functions which can be very different in different types of cells. However, the prostaglandins also play critical roles in pathophysiology. In particular, inflammation is both initiated and maintained, at least in part, by the overproduction of prostaglandins in injured cells. The central role that prostaglandins play in inflammation is underscored by the fact that those aspirin-like non-steroidal anti-inflammatory drugs (NSAIDS) that are most effective in the therapy of many pathological inflammatory states all act by inhibiting prostaglandin synthesis.

A widespread super-family designated MAPEG (Membrane Associated Proteins in Eicosanoid and Glutathione metabolism) has been defined according to enzymatic activities, sequence motifs and structural properties (Jakobsson et al. Prot. Sci., 8, 689, 1999 and Jakobsson et al. Am. J. Respir. Crit. Care Med., 161, S20, 2000). The family consists of six human proteins including 5-lipoxygenase activating protein (FLAP), LTC₄ synthase, microsomal glutathione S-transferase (MGST)1, MGST2 (Jakobsson, et al., J. Biol. Chem., 271, 22203, 1996 and Scoggan et al., J. Biol. Chem., 272, 10182, 1997), MGST3 Jakobssen et al., J. Biol. Chem., 272, 22934, 1997) and MGST1-like 1, now identified as prostaglandin (PG) E synthase (Jakobssen et al., Proc. Natl. Acad. Sci. USA, 96(13), 7220, 1999).

FIG. 1 illustrates the metabolic routes of arachidonic acid, which are relevant for the MAPEG members, i.e. the 5-lipoxygenase and cyclooxygenase pathways. In the presence of FLAP, 5-lipoxygenase catalyzes the formation of the unstable epoxide leukotriene (LT) A₄ (Samuelsson, B., Science, 220, 568, 1983). This reaction proceeds via the intermediate 5-hydroperoxy-eicosatetraenoic acid (5-HpETE). At an excess of 5-HpETE production, MGST2 and/or MGST3 may metabolize this product by reduction to the corresponding alcohol, 5-HETE (Jakobsson et al., J. Biol. Chem., 272, 22934, 1997). One important metabolic route of LTA₄ is the conjugation with reduced glutathione, leading to the formation of LTC₄. There are three known enzymes that may catalyze this reaction, i.e. LTC₄ synthase, MGST2 or MGST3 (Jakobsson et al, J. Biol. Chem., 271, 22203, 1996; Scoggan et al., J. Biol. Chem., 272, 10182, 1997; and Jakobsson et al., J. Biol. Chem., 272, 22934, 1997). The product, LTC₄ and its metabolites LTD₄ and LTE₄ are potent constrictors of human bronchi and during the last year Singulair™ (a CysLT1 receptor antagonist) has been introduced for the treatment of patients suffering from various types of asthma. In essence, both MGST2 and MGST3 have been characterized to constitute microsomal glutathione S-transferases and peroxidases (Jakobsson et al, J. Biol. Chem., 271, 22203, 1996 and Jakobsson et al., J. Biol. Chem., 272, 22934, 1997). Their importance for the production of LTC₄ is currently being investigated by persons of skill in the art. Since MGST2 and MGST3, in comparison to LTC₄ synthase, possess a broader substrate specificity as well as an additional catalytic property (peroxidase activity) these enzymes may also be involved in the detoxification of various xenobiotics. Their role in physiology is being further investigated.

Prostaglandin endoperoxide H₂ (PGH₂) is formed from arachidonic acid by the action of cyclooxygenases (COX)-1 or -2 (FIG. 1). COX-1 is constitutively expressed in many cells and tissues whereas the COX-2 protein can be induced by proinflammatory cytokines such as interleukin-1β at sites of inflammation. Downstream of the cyclooxygenases, the product PGH₂ can be further metabolized into various physiologically important eicosanoids e.g. PGF_{2α}, PGE₂, PGD₂, PGI₂ (prostacyclin) and thromboxane (TX) A₂ (Smith, W. L., Am. J. Physiol., 263, F181, 1992). Regarding inflammation, prostaglandin E₂ is considered to be the most important prostaglandin mediator. This fact has recently been supported by experiments with knock out mice lacking one of the PGE receptors (EP3). These mice completely failed to respond with fever triggered by interleukin-1β and lipopolysaccharide (Ushikubi et al., Nature, 395, 281, 1998). In addition, PGE₂ seems to play a key role in inflammatory pain since a selective anti-PGE₂ antibody was shown to inhibit, equally potent as indomethacin (a NSAID), phenylbenzoquinone-induced writhing in mice and carrageenan-induced paw oedema and hyperalgesia in rats (Mnich et al., J. Immunol., 155(9), 4437, 1995 and Portanova et al., J. Exp. Med., 184(3), 883, 1996).

PGE₂-sensitive receptors have been sub-divided into four subtypes, EP1, EP2, EP3 and EP4, and these receptors have a wide distribution in various tissues. The effects associated with EP1 and EP3 receptors may be considered as excitatory, and are believed to be mediated by stimulation of phosphatidylinositol turnover or inhibition of adenyl cyclase activity, with resulting decrease in intracellular levels of cyclic AMP. In contrast, the effects associated with EP2 and EP4 receptors may be considered as inhibitory, and are believed to be associated with a stimulation of adenyl cyclase and an increase in levels of intracellular cyclic AMP. Especially, EP4 receptor may be considered to be associated with smooth muscle relaxation, anti-inflammatory or pro-inflammatory activities, lymphocyte differentiation, antiallergic activities, mesangial cell relaxation or proliferation, gastric or enteric mucus secretion, or the like.

Accordingly, it would be desirable to have methods and compositions for controlling the production and activity of prostaglandins, and to be able to prepare prostaglandins in vitro. However, despite many attempts to purify and characterize PGE synthase, the enzyme has not been purified sufficiently to permit these uses.

The contents of all cited references including literature references, issued patents, published patent applications as cited throughout this application, including the foregoing, are hereby expressly incorporated by reference in their entirety.

### SUMMARY OF THE INVENTION

The invention provides substantially pure preparations of prostaglandin E synthase (PGES) and methods of obtaining these pure preparations. PGES can be fused to a heterologous polypeptide. In a preferred embodiment, the PGES is a human PGES, e.g., having SEQ ID NO: 2, or an analog or variant thereof, such as a portion of a PGES. The invention also provides nucleic acids encoding PGES polypeptides.

Purified polypeptides or fusion polypeptides have preferably less than about 10% contaminating cellular material; even more preferably less than about 5% contaminating cellular material; less than about 1% and even more preferably less than about 0.1% contaminating cellular material. Purified polypeptides or fusion polypeptides are preferably substantially free of other cellular proteins, and have, e.g., less than about 10% contaminating proteins; preferably less than about 5% contaminating proteins; and even more preferably, less than about 1% or 0.1% contaminating proteins.

Purified PGES polypeptides or fusion polypeptides or analogs or variants thereof are preferably capable of catalyzing the conversion of a cyclic endoperoxide substrate into the 9-keto, 11α hydroxy form of the substrate, e.g., the converstion of PGH₁ into PGE₁; PGH₂ into PGE₂ and PGH₃ into PGE₃. Purified polypeptides preferably have a specific activity of at least about 100 μmol/min/mg, when the activity is measured at about 37° C. or at least about 10 μmol/min/mg, when the activity is measured at about 0° C.

In a preferred embodiment, PGES polypeptides or analogs or variants thereof are fused in frame to one or more heterologous polypeptides which mediate binding of the fusion polypeptides to affinity matrices, such as a metal ion affinity matrix, e.g., a Ni²⁺ chelate affinity matrix. A heterologous polypeptide can be, e.g., a polypeptide comprising or consisting of from one to twelve consecutive histidine residues, and preferably six histidine residues. A preferred fusion polypeptide comprises the amino acid sequence set forth in SEQ ID NO: 4 and shown in FIG. 2A.

Preparations of PGES or fusion polypeptides or analogs or variants thereof can comprise about 10% glycerol and/or about 1-5 mM reduced glutathione. Preparations can be in a form of a liquid or frozen.

The invention further provides methods for obtaining substantially pure preparations of PGES fusion polypeptides or analogs or variants thereof, comprising, e.g., (i) providing a lysate comprising a PGES fusion polypeptide comprising a PGES polypeptide, analog or fragment thereof fused to a heterologous polypeptide capable of binding to a metal ion affinity chromatography; (ii) subjecting the lysate of (i) to hydroxyapatite (HA) chromatography; (iii) obtaining the unbound fraction from the HA chromatography; (iv) subjecting the unbound fraction from the HA chromatography to metal ion affinity chromatography, in conditions appropriate for the fusion polypetide to bind to the metal ion; and (v) eluting the metal ion affinity chromatography, to thereby obtain a substantially pure preparation of a PGES fusion polypeptide. The lysate can be a whole cell lysate or a solubilized membrane fraction of a cell. The lysate can be from a prokaryotic cell, e.g., an E. coli bacterium, or from a eukaryotic cell.

One or more of the steps of the method, e.g., the HA chromatography or the metal affinity chromatography, can be performed in the presence of about 10% glycerol and/or about 1-5 mM reduced glutathione. In a preferred embodiment, each step of the method is performed in solutions comprising about 10% glycerol and/or about 1-5 mM reduced glutathione.

In a preferred embodiment of the invention, the metal affinity chromatography is a Ni²⁺ affinity chromatography, and PGES polypeptides are fused to tags comprising from one to six consecutive histidine residues.

Fusion polypeptides optionally comprise a protease recognition site between the PGES polypeptides or analogs or variants thereof and the heterologous polypeptides, and the method further comprises subjecting the fusion polypeptides to the protease recognizing the protease recognition site after the metal ion affinity chromatography, such that the heterologous polypeptides are cleaved from the PGES polypeptides or analogs or fragments thereof.

Purified PGES fusion polypeptides or purified PGES polypeptides or analogs or fragments thereof prepared by the methods of the invention are also within the scope of the invention. Pharmaceutical compositions comprising the above-described PGES preparations are also provided by the invention.

The invention further provide methods for identifying an agent which modulates the interaction between a PGES polypeptide or analog or fragment thereof and a PGES-binding partner, comprising contacting a substantially pure PGES polypeptide or analog or fragment thereof; a PGES-binding partner; and a test agent, in conditions under which, but for the presence of the test agent, the PGES polypeptide or analog or fragment thereof and the PGES binding partner interact, wherein a difference in the interaction between the PGES polypeptide or analog or fragment thereof and the PGES-binding partner indicates that the test agent modulates their interaction. PGES polypeptides or analogs or fragments thereof can be fused to one or more heterologous polypeptides which bind a metal ion affinity chromatography to form fusion polypeptides.

Also provided by the invention are methods for in vitro preparing a prostaglandin or analog thereof. The invention provides methods for converting any molecule having a cyclic endoperoxide into a molecule having a 9-keto, 11-alpha-hyddroxy. For example, PGH₁, PGH₂ and PGH₃ and analogs thereof, can be converted into PGE₁, PGE₂ and PGE₃ and analogs thereof, respectively. The methods may comprise contacting a PGH molecule and a substantially pure preparation of PGES or analog of fragment thereof or fusion polypeptide and reduced glutathione, under conditions suitable for the catalysis of the conversion of PGH into PGE by PGES.

PGE or purified PGES or modulators thereof can be used for treating a subject in need of modulating prostaglandin effects. For example, purified preparations of the invention, e.g., PGE or purified PGES or modulators thereof can be used to treat PGE or PGE-related diseases, including, e.g., inflammatory diseases, pain, blood pressure, uterine contractile activity, and bone resorption. An antagonist of PGES can be, e.g., a dominant negative mutant of naturally-occurring PGES.

Yet also provided by the invention are methods for determining the three dimensional structure of a PGES polypeptide or analog or fragment thereof, comprising crystallizing a preparation of purified PGES or fusion polypeptide; determining the coordinates of the crystal structure; and introducing the coordinates into a computer program allowing the determination of three dimensional structures from coordinates. The knowledge of the three dimensional structure of PGES allows the selection of drugs that interact with PGES.

### BRIEF DESCRIPTION OF THE DRAWINGS

FIG. 1 shows the MAPEG members (in bold) and their role in eicosinoid metabolism.

FIG. 2A shows a preferred embodiment of a PGES polynucleotide and polypeptide consisting of the nucleotide and amino acid sequences of His₆-PGES used in the Examples.

FIG. 2B (sheets 1-4) shows a preferred embodiment of a vector comprising a nucleotide sequence of the invention consisting of the nucleotide sequence of the vector h6pSP19T7LThPGES containing the nucleotide sequence of FIG. 2B encoding His₆-hPGES.

FIG. 2C represents a Western blot of a membrane preparation from bacteria expressing recombinant PGES suspended in 50 mM sodium phosphate buffer, pH 8, 150 mM NaCl, and solubilized in 4% Triton X-100. The membrane suspension was solubilized for 30 minutes on ice and insoluble particles were removed by ultracentrifugation. Samples were subjected to gel electrophoresis and Western blotting in the amounts indicated in the figure. Unsolubilized sample was compared with the solubilized sample as indicated before and after removal of insoluble material in order to estimate the degree of extraction of PGES.

FIG. 3 is a graph showing the PGES activity in bacterial membrane fractions expressing recombinant PGES solubilized in different buffer compositions relative to the amount of membrane protein included in the reaction. Membranes were solubilized by 4% Triton X-100 in a buffer consisting of 50 mM sodium phosphate, pH 8, 150 mM NaCl (filled circles), buffer+10% glycerol (open circles), buffer+1 mM reduced glutathione (GSH) (filled triangles) and buffer+1 mM GSH+10% glycerol (open triangles).

FIG. 4 shows the stability over time of PGES activity in solubilized bacterial membranes stored at 4° C. in the presence of 1 mM GSH and 10% glycerol. Membrane fraction from bacteria expressing recombinant PGES was solubilized by 4% Triton X-100 in 50 mM sodium phosphate, pH 8, 150 mM NaCl, 1 mM GSH, 10% glycerol and stored at 4° C. Samples were withdrawn at the indicated time points and PGES activity assayed with 0.25 mg/ml membrane protein in the reaction.

FIG. 5A is a chromatogram from Ni-affinity purification of his₆-hPGES, which was eluted with 350 mM imidazole.

FIG. 5B is a silver stained 15% SDS-PAGE gel with protein samples from an experiment with purification of PGES from solubilized bacterial membrane extract. Membrane fractions from bacteria expressing recombinant, histidine-tagged PGES were solubilized and PGES was purified as described in Example 3 below.

Lane 1: Molecular weight markers, from bottom: 9.0, 16.2, 21.8, 27.7, 39.8, 52.8 and 64.9 kDa.

Lane 2: Positive control, purified PGES from an earlier experiment, 0.125 μg

Lane 3: Protein peak eluted with 60 mM imidazole from first Ni-column, 10 μl

Lane 4: Protein peak eluted with 300 mM imidazole from first Ni-column, 0.19 μg

Lane 5: Protein peak eluted with 300 mM imidazole from second Ni-column, 0.13 μg

Lane 6: Protein peak eluted with 300 mM imidazole from second Ni-column, desalted on a PD10 column, 0.14 μg

FIG. 6 shows a quantitative Western blot where indicated amounts of purified PGES were blotted as a standard curve for determination of the PGES content in bacterial membranes. The band intensity was determined by densitometry and the increase was linear up to 20 ng of purified PGES.

FIG. 7 shows the enzymatic activity of purified PGES and the effect of storage under different conditions. The purpose of the experiment was to investigate the stability of purified PGES and the possibilities of storage. Purified PGES at the indicated concentrations was incubated with 10 μM PGH₂ and 2.5 mM GSH at 0° C. and the enzymatic formation of PGE₂ was analyzed as described in Example 2 below. Reactions were performed with freshly purified PGES (open triangles), protein stored for 5 days at 4° C. (filled circles), stored frozen at −20° C. and thawed before the experiment (open circles) and corresponding amounts of unsolubilized PGES (filled triangles) (bacterial membrane fraction containing 0.56% recombinant PGES as determined by quantitative Western blots).

FIG. 8 is a silver stained 15% SDS-PAGE gel with protein samples from an experiment with purification of PGES from bacterial whole cell lysate. A whole cell lysate was prepared from E. coli bacteria expressing recombinant, histidine-tagged PGES. The lysate was solubilized and PGES purified as described in Example 7 below.

Lane 1: Molecular weight markers, from bottom: 9.0, 16.2, 21.8, 27.7, 39.8, 52.8 and 64.9 kDa.

Lane 2: Solubilized whole cell extract, 0.1 μl Lane 3: Unbound fraction from the hydroxyapatite chromatography step, 0.11 μl

Lane 4: Flow-through fraction from the Ni-column, 0.2 μl

Lane 5: Protein peak eluted with 60 mM imidazole from the Ni-column, 0.2 μl

Lane 6: Protein peak eluted with 300 mM imidazole from the Ni-column, 0.16 μg

Lane 7: Protein peak eluted with 500 mM imidazole from the Ni-column, 0.2 μg

Lane 8: Positive control, purified PGES from an earlier experiment, 0.125 μg

FIG. 9 shows a silver stained 15% SDS-PAGE gel with protein samples from a purification scale-up test from bacterial whole cell lysate. A whole cell lysate consisting of 5

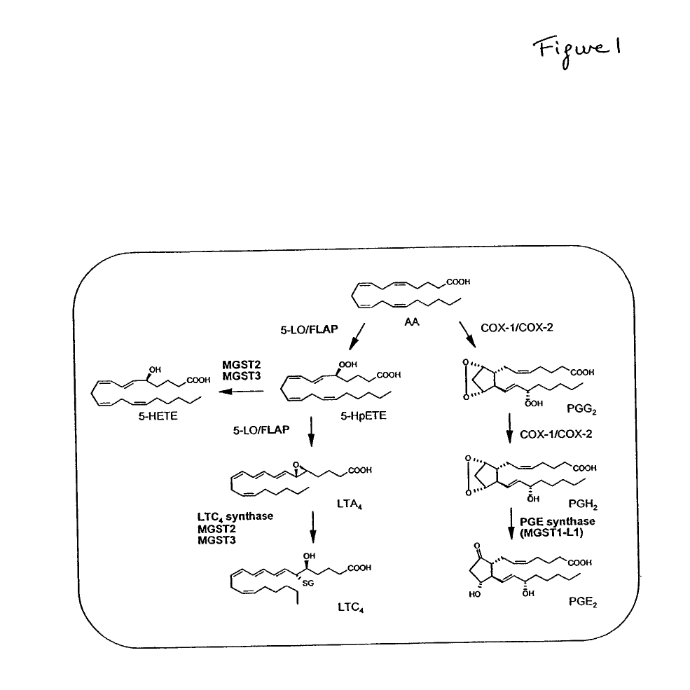

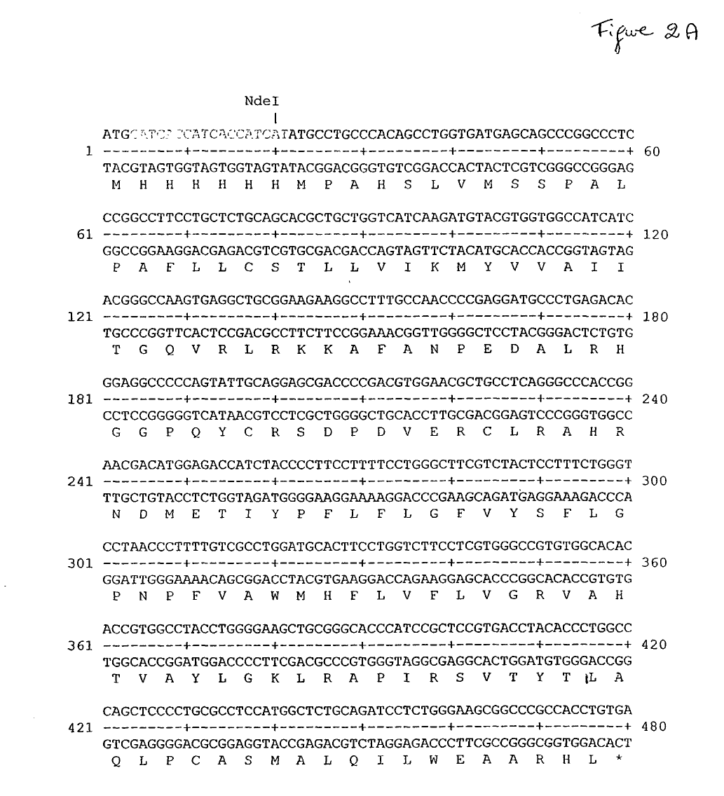

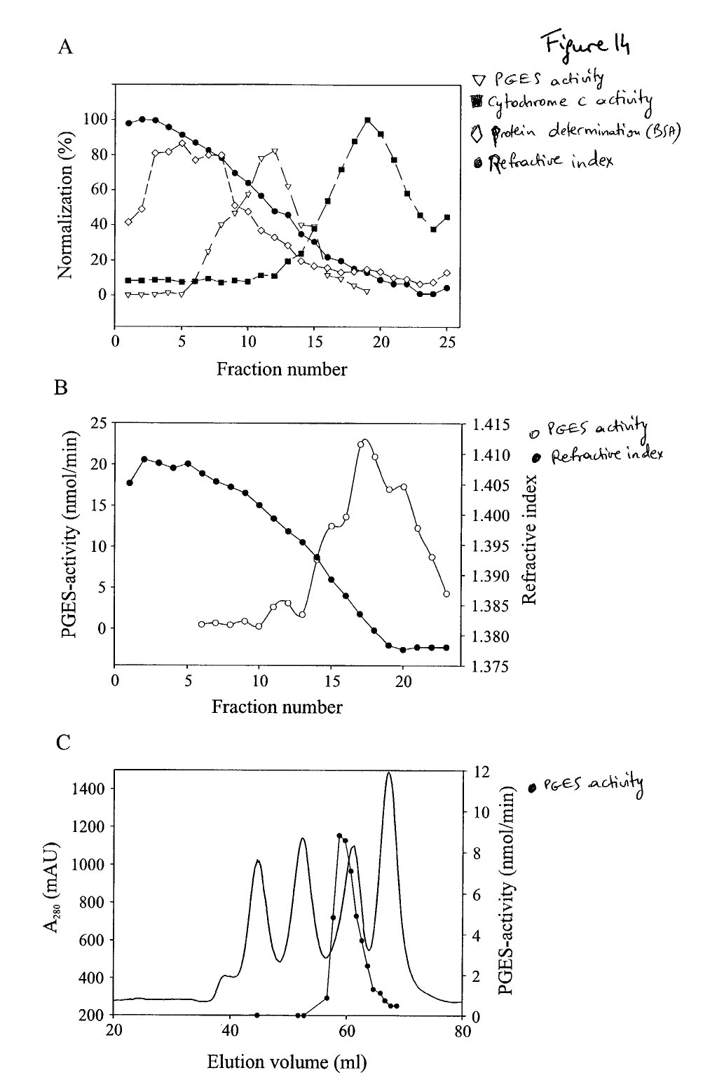

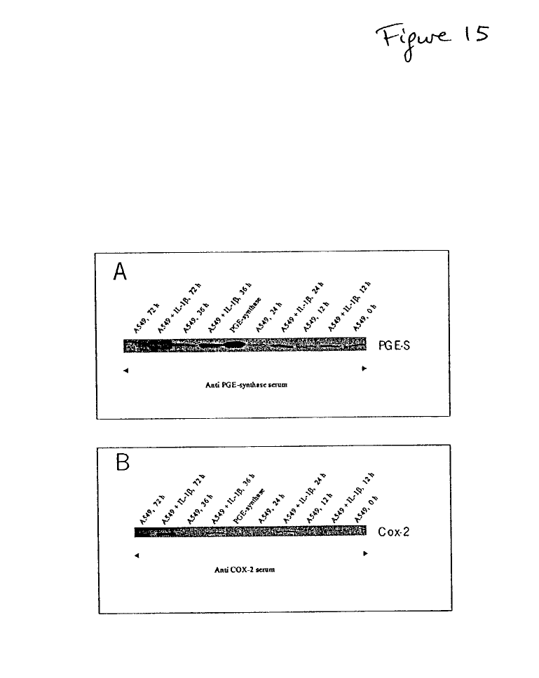

times more material than is described in Example 7 and FIG. 8 was prepared from E. coli bacteria expressing recombinant, histidine-tagged PGES (51 culture). The lysate was solubilized and PGES purified using 5 times larger column sizes as described in Example 8 below.

Lane 1: Molecular weight markers, from bottom: 9.3, 13.1, 19.2, 24.7, 36.4, 49.0 and 61.3 kDa.

Lane 2: Positive control, purified PGES from an earlier experiment, 0.125 μg

Lane 3: Protein peak eluted with 350 mM imidazole from the first, larger (5 ml), Ni-column, 0.4 μg

Lane 4: Protein peak eluted with 350 mM imidazole from a second, (1 ml size), Ni-column, 0.2 μg

Lane 5: Protein peak eluted with 350 mM imidazole from a third, (ml size), Ni-column, 0.21 μg

Lane 6: Flow-through fraction from a fourth, (1 ml size), Ni-column, 0.1 μl

Lane 7: Protein peak eluted with 350 mM imidazole from the fourth Ni-colunm, 0.2 μg

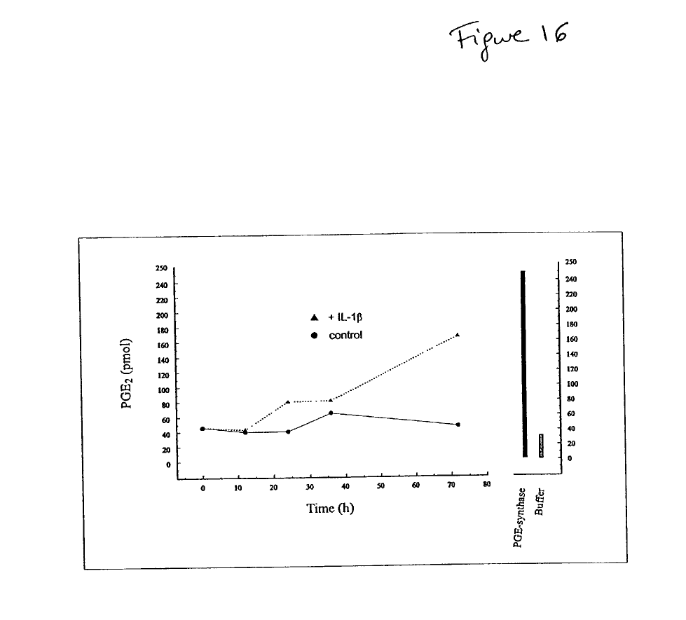

FIG. 10 is a Western blot of protein peaks from the purification scale-up test described in Example 8 and silver stained as shown in FIG. 9. Proteins were separated by SDS-PAGE, transferred to a PVDF membrane and the PGES was immunodetected as described in Example 1.

Lane 1: Molecular weight markers, from bottom: 13.1, 19.2, 24.7, 36.4, 49.0 and 61.3 kDa.

Lane 2: Positive control, purified PGES from an earlier experiment, 50 ng

Lane 3: Solubilized whole cell lysate, 10 μg

Lane 4: Protein peak eluted with 350 mM imidazole from the first, larger (5 ml), Ni-column, 50 ng

Lane 5: Protein peak eluted with 350 mM imidazole from the second, (1 ml size), Ni-column, 50 ng

Lane 6: Protein peak eluted with 350 mM imidazole from the third, (1 ml size), Ni-column, 50 ng

Lane 7: Flow-through fraction from the fourth, (1 ml size), Ni-column, 0.05 μl

Lane 8: Protein peak eluted with 350 mM imidazole from the fourth Ni-column, 50 ng

Lane 9: Flow-through fraction from the first Ni-column, 0.1 μl

FIG. 11A shows V vs. [s] curves determined with 1 μg/ml purified PGES in the presence of 2.5 mM GSH and varying PGH₂ concentrations at 0° C. Km for PGH₂ was determined to be 50±8.9 μM and Vₘₐₓ was 0.25±0.013 μM s⁻¹.

FIG. 11B shows V vs. [s] curves determined with 1 μg/ml purified PGES in the presence of 2.5 mM GSH and varying PGH₂ concentrations at 37° C. Km for PGH₂ was determined to be 163±421M at 37° C. and Vₘₐₓ was 1.39±0.16 μM s⁻¹ at 37° C.

FIG. 11C shows V vs. [s] curves determined with 1 μg/ml purified PGES in the presence of 2.5 mM GSH and varying PGG₂ concentrations at 37° C.

FIG. 12 shows chromatograms of 15-HP PGE₂ synthase activity at 195 nm by purified His₆-hPGES (20 ng) and buffer controls after incubation with 10 μM PGG₂ in presence of 2.5 mM GSH at 37° C. FIG. 12A shows an exemplary chromatogram containing known prostaglandins, as a control. FIG. 12B shows the products of a reaction mixture containing purified His₆-hPGES after 30 seconds incubation. FIG. 12C shows the products of a reaction mixture containing a buffer control after 30 seconds incubation. FIG. 12D shows the products of a reaction mixture containing purified His₆-hPGES after 10 minutes incubation. FIG. 12E shows the products of a reaction mixture containing a buffer control after 10 minutes incubation. FIG. 12F shows the products of a reaction mixture containing His₆-hPGES after 30 seconds incubation in the presence of 10 μM PGG₂, but in the absence of GSH.

FIG. 13 shows a projection map of his₆-hPGES calculated from a data set merged from 18 unstained crystalline areas. For one one of the found unit cells the glide lines along the a- and b-axes and the two-fold rotation of the pgg projection symmetry have been depicted. Two protein units corresponding to two trimers of the protein in opposite orientations relatively to the membrane plane have been encircled.

FIG. 14A shows the determination of the sedimentation coefficient of the PGES-Triton complex by sucrose gradient centrifugation (5-20% sucrose).

FIG. 14B represents equilibrium density gradient centrifugation fo the PGES-Triton X-100 complex.

FIG. 14C represents the elution of the PGES-Triton X-100 complex and marker enzymes from Sephacryl X-300 HR. 1 ml fractions were assayed for PGES activity. The absorbance at 280 nm was monitored continuously as demonstrated by a plain line.

FIG. 15 is a Western blot analysis of the expression of PGES (FIG. 12A) and COX-2 (FIG. 12B) in microsomal fractions of A549 cells cultured without or in the presence of IL-1β (1 ng/ml) for the indicated time as described in Example 13 below.

FIG. 16 shows a preferred embodiment of PGES enzymatic activity, which is a time-course of PGES enzymatic activity in microsomes isolated from A549 cells cultured in the absence or presence of IL-1β (1 ng/ml) for the indicated time. The black bar indicates the corresponding PGE₂ formation measured in membranes from bacteria expressing recombinant PGES. The gray bar indicates non-enzymatic PGE₂ formation in the buffer. Details are described in Example 13.

### DETAILED DESCRIPTION OF THE INVENTION

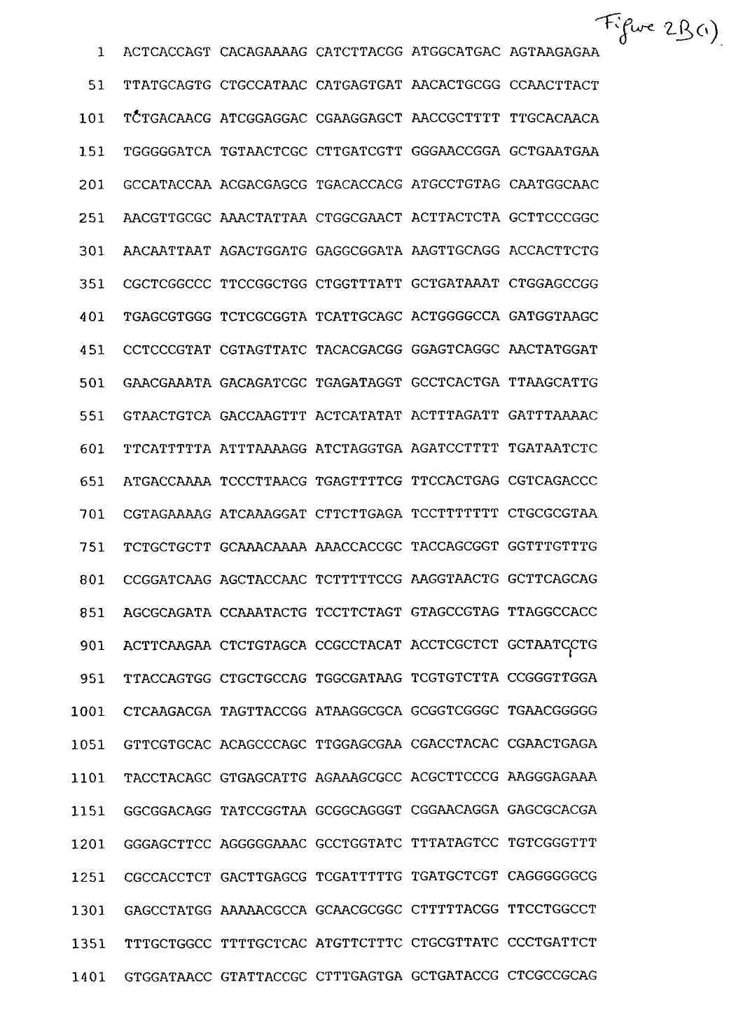

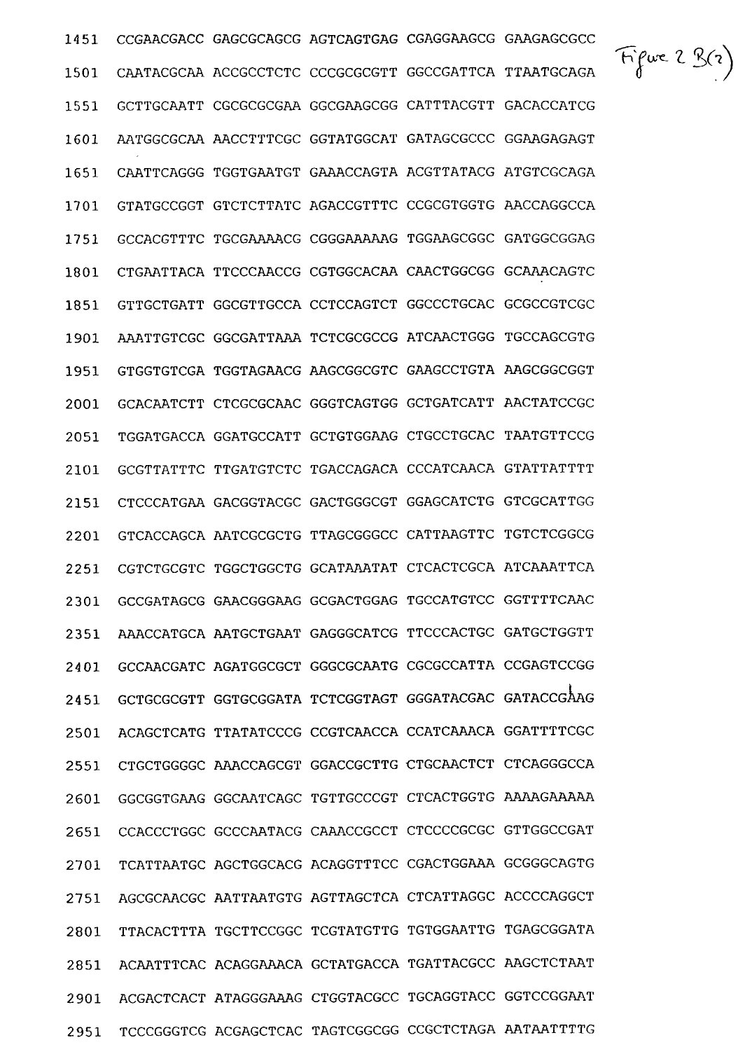

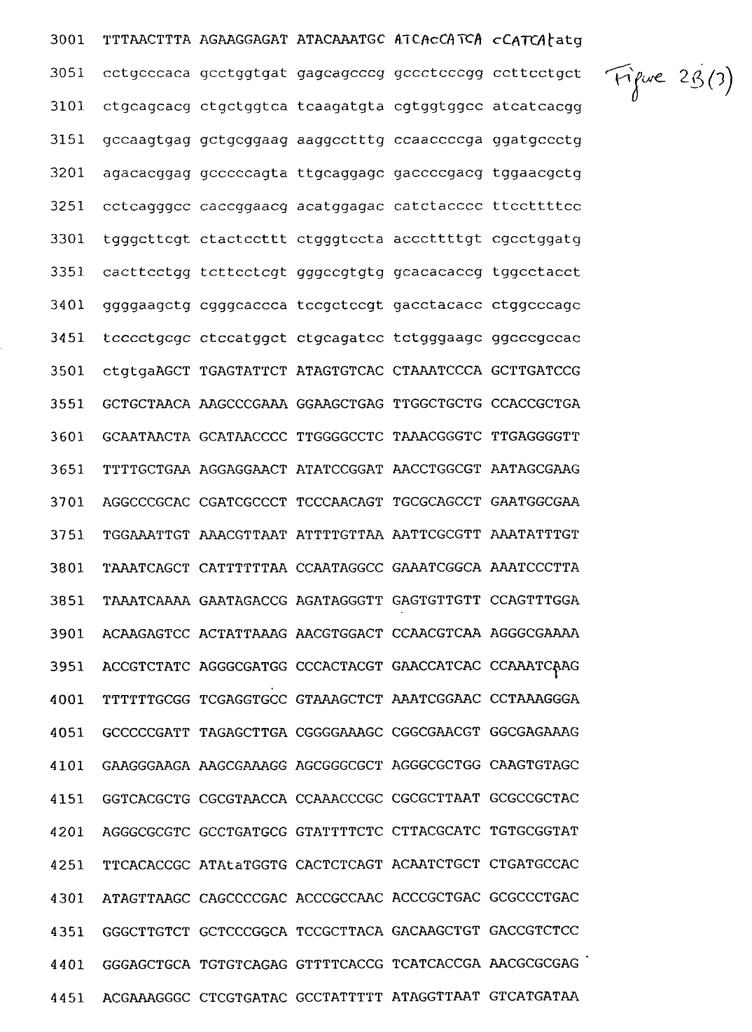

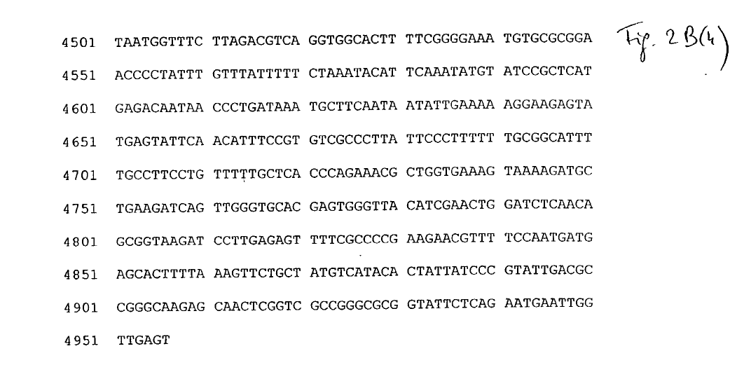

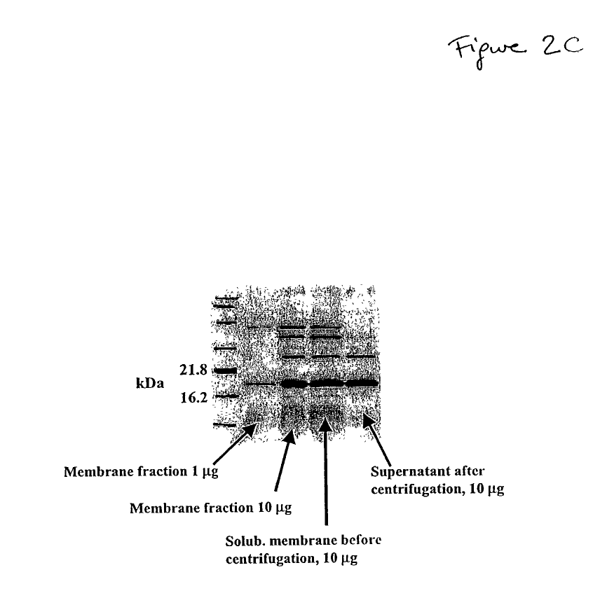

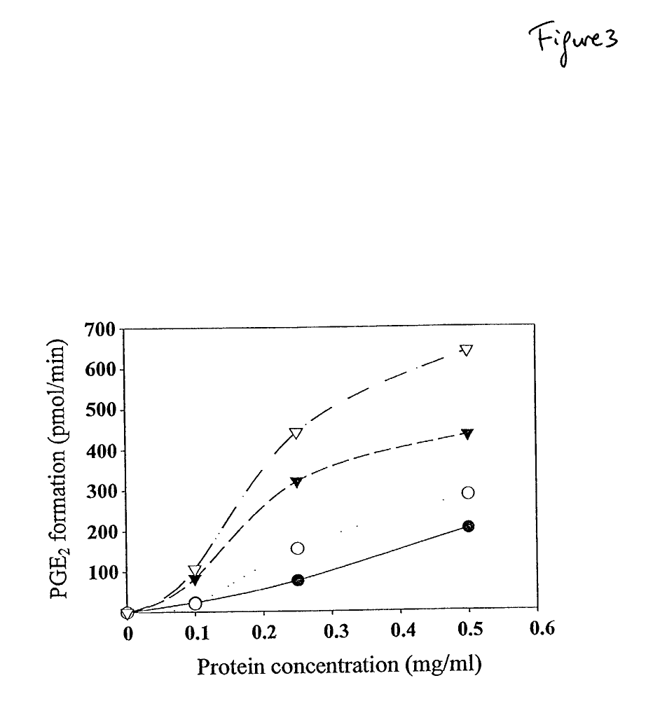

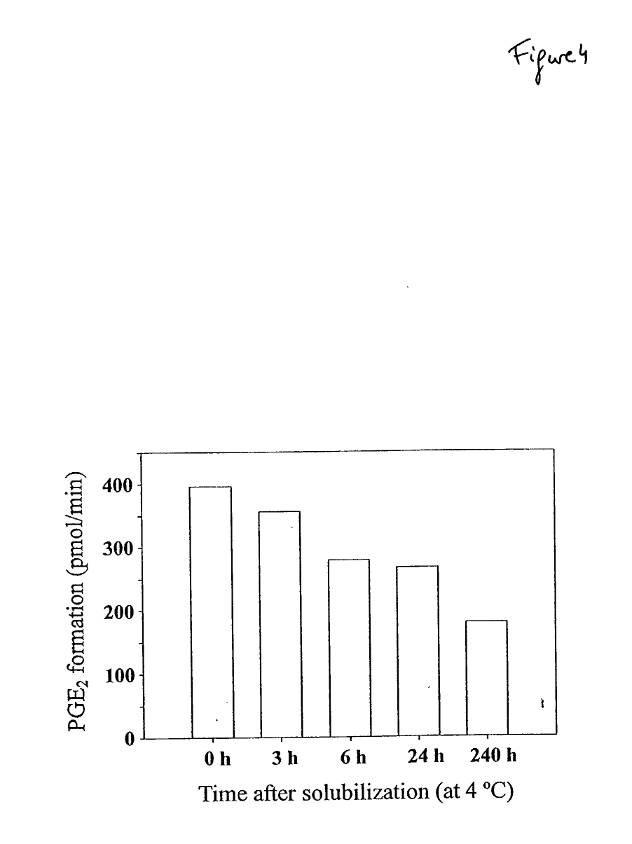

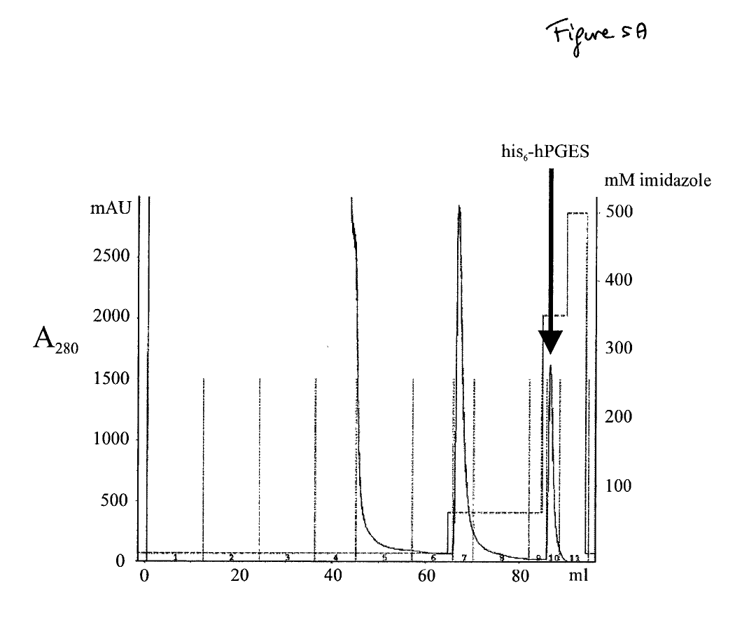

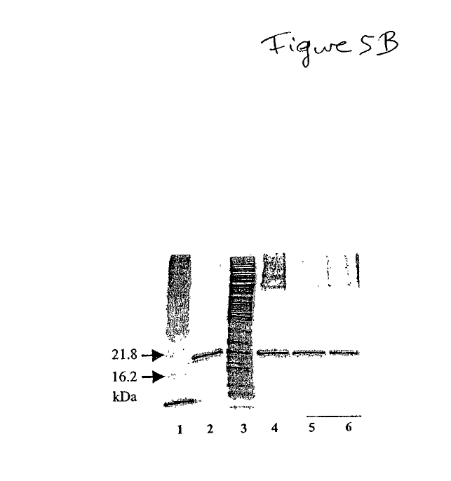

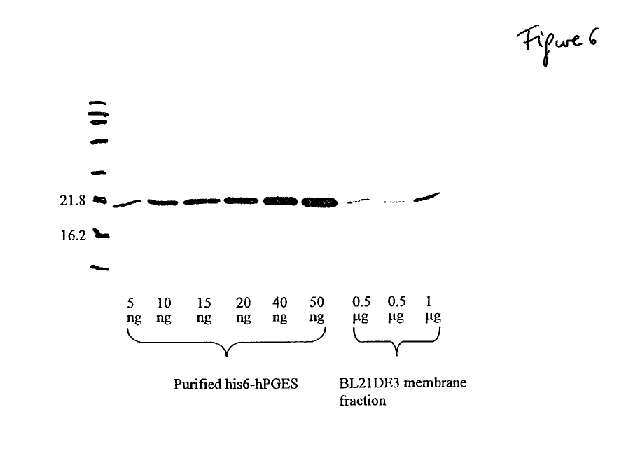

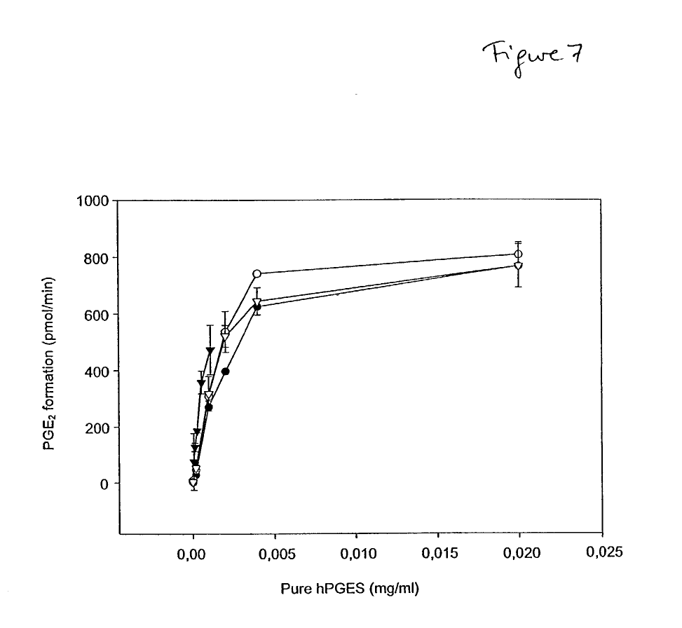

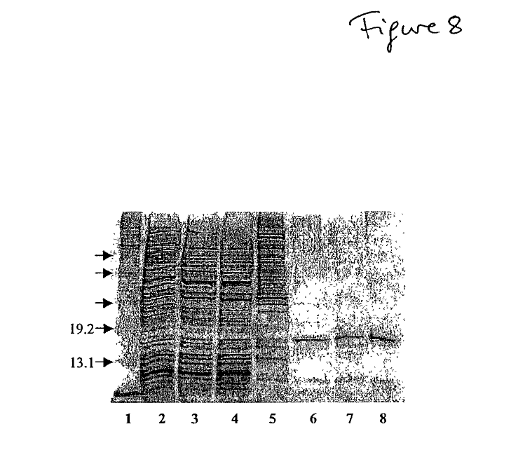

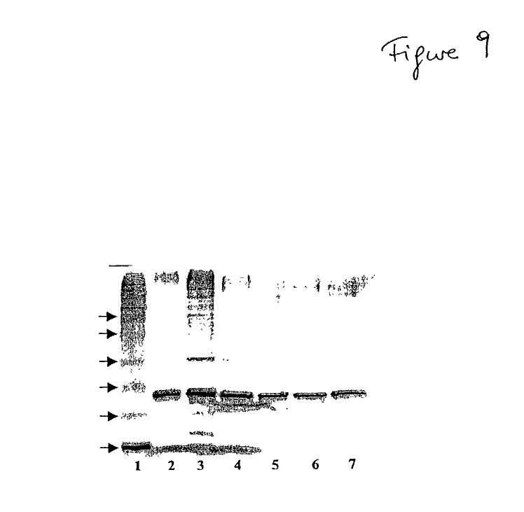

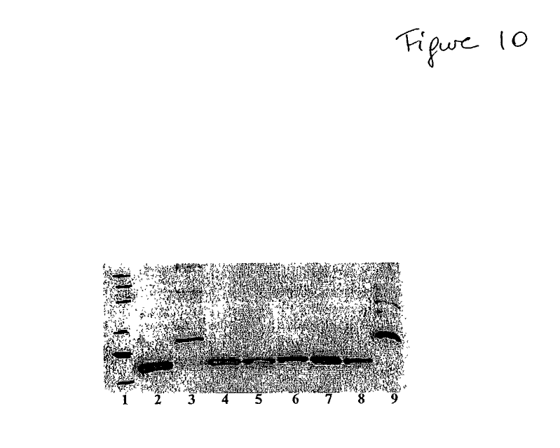

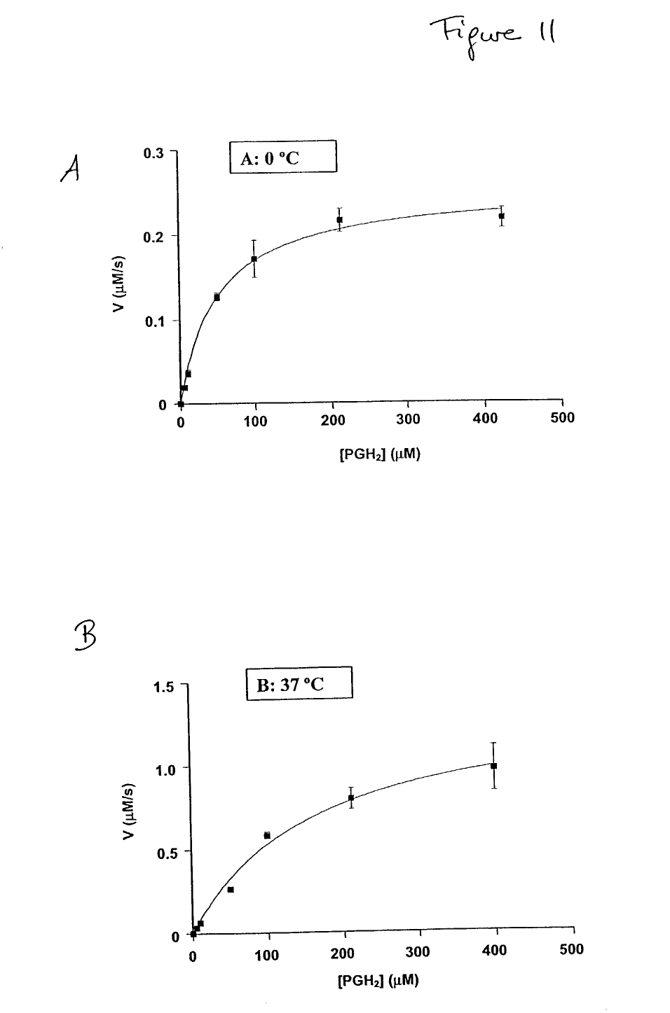

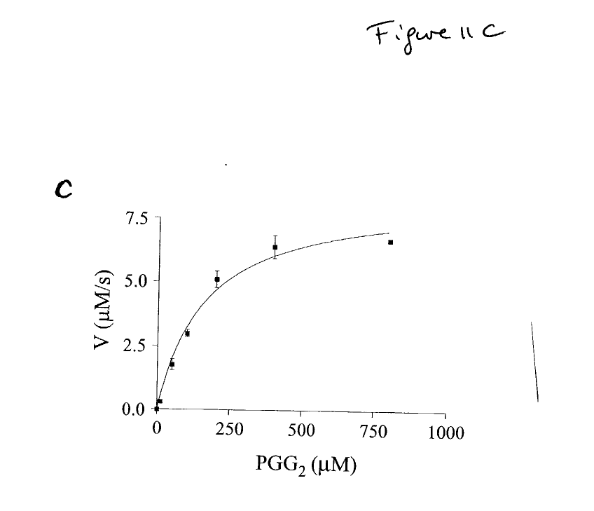

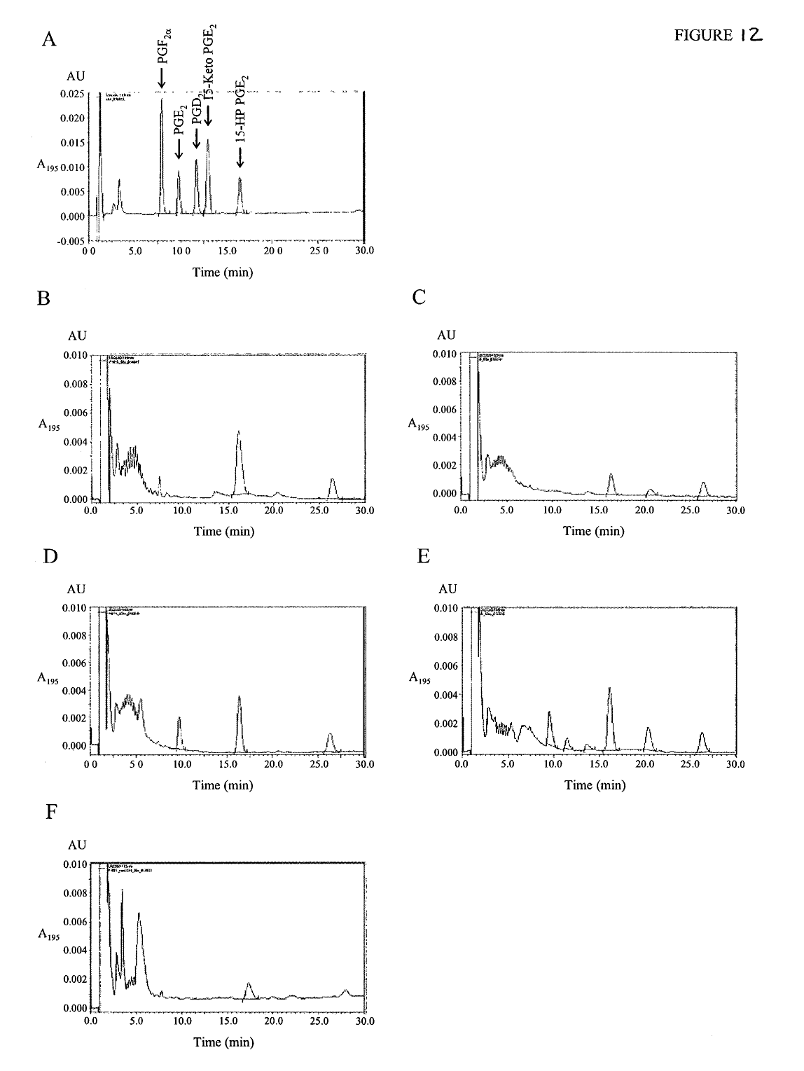

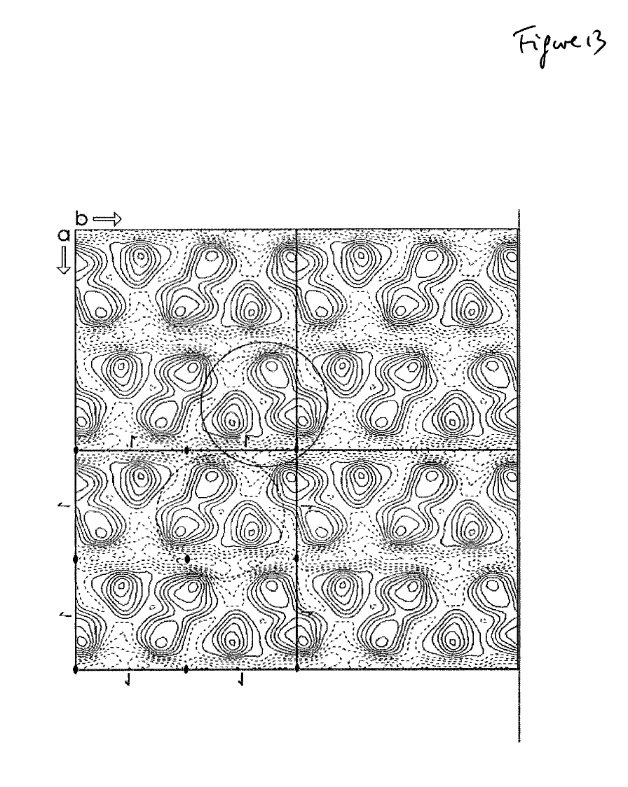

The invention is based at least on the discovery of a method that allows the preparation of highly pure PGE synthase (PGES). Several figures are provided that help to further describe the invention. A person of skill in the art will appreciate from FIG. 2C, that recombinant PGES can be efficiently extracted from the bacterial membrane and solubilized in the described buffer composition without loss of protein; from FIG. 3, that the preservation of PGES enzymatic activity during/after solubilization is improved by the presence of GSH and glycerol; from FIG. 4, that the activity of solubilized PGES is preserved for a prolonged time by the presence of GSH and glycerol (in the absence of GSH and glycerol all detectable activity is lost within a few hours); from FIG. 5, that recombinant PGES can be purified to apparent homogeneity from solubilized membrane fractions by the method described in Example 3; from FIG. 6, that the amount of PGES in a crude fraction can be determined with quantitative Western blots using purified protein as standard; from FIG. 7, that the enzymatic activity of the purified PGES in solution is equal to the activity determined in unsolubilized membranes, demonstrating that the protein can be solubilized and purified in a functional state using the actual method, and that the purified, solubilized PGES is resistant to freeze-thawing without loss of enzymatic activity thus allowing for long-term storage of the protein in the actual buffer/detergent composition; from FIG. 8, that recombinant PGES can also be purified to apparent homogeneity from bacterial whole cell lysate without first isolating the membrane fraction; from FIG. 9, that recombinant PGES can be purified to apparent homogeneity from a larger amount of material using correspondingly larger columns as described in Example 8, and that the recovery of purified PGES; from FIG. 12, that the expression of PGES is induced by IL-1β in concordance with an up-regulation of COX-2; from FIG. 13, that the increase of microsomal PGES enzymatic activity upon incubation with IL-1β is proportional to the induction of protein expression.

The terms used herein have their usual meaning in the art, however, to even further clarify the present invention, for convenience, the meaning of certain terms and phrases employed in the specification, examples, and appended claims are provided below.

“PGES” or “PGE synthase” refers to Prostaglin E synthase, an enzyme that catalyzes the conversion of a cyclic endoperoxide substrate into a 9-keto, 11 a hydroxy form of the substrate. PGES catalyzes, e.g., the conversion of precursor molecules into PGE₁, PGE₂ and PGE₃ and analogs thereof, e.g., synthetic analogs. For example, PGES catalyzes the conversion of PGH₂ into PGE₂.

A “PGES substrate” is any molecule having a cyclic endoperoxide structure, which can be converted into a 9-keto, 1 la hydroxy form by PGES. Exemplary PGES substrates include PGH₁, which is converted by PGES into PGE₁; PGH₂, which is converted by PGES into PGE₂; PGH₃, which is converted by PGES into PGE₃; PGG₁, which is converted by PGES into 15 (S) hydroxyperoxy PGE₁; PGG₂, which is converted by PGES into 15 (S) hydroxyperoxy PGE₂; and PGG₃, which is converted by PGES into 15 (S) hydroxyperoxy PGE₃. (see WO 00/28022).

“PGES nucleic acid” is a nucleic acid encoding a PGES polypeptide, as defined herein.

“PGES agonist” refers to an agent that mimics PGES bioactivity.

“PGES antagonist” refers to an agent that inhibits PGES bioactivity, such as by competing with wild-type PGES.

“Wild-type PGES” refers to a naturally-occurring PGES gene (or polypeptide encoded thereby) which is the most common form (i.e., allele) of the gene.

“PGES polypeptides or analogs or fragments thereof” is intended to include PGES polypeptides, analogs of PGES polypeptides; fragments of PGES polypeptides and fragments of PGES analogs.

“Biological activity” or “bioactivity” or “activity” or “biological function”, which are used interchangeably, for the purposes herein means an effector function performed by a PGES polypeptide, e.g., the ability to catalyze the formation of PGE₂ from PGH₂.

“Bioactive fragment of a PGES polypeptide” refers to a fragment of a full-length PGES polypeptide, wherein the fragment specifically mimics or antagonizes the activity of a wild-type PGES polypeptide.

“Cells,” “host cells” or “recombinant host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein. Cells include eukaryotic and prokaryotic cells. Preferred cells are E. coli bacteria.

A “chimeric polypeptide” or “fusion polypeptide” is a fusion of a first amino acid sequence encoding a PGES polypeptide or analog or fragment thereof with a second amino acid sequence that is different from (i.e., heterologous to) the PGES polypeptide, and referred to herein as “heterologous polypeptide” or “foreign polypeptide.” The PGES polypeptide or analog or fragment thereof can be located at, e.g., fused to, the N-terminus or at the C-terminus end of the heterologous polypeptide. The heterologous polypeptide can also be internal to the PGES polypeptide. A fusion polypeptide may present a foreign domain which is found in an organism which also expresses the first polypeptide (albeit in a different polypeptide), or it may be an “interspecies”, “intergenic”, etc. fusion of polypeptide structures expressed by different kinds of organisms. In a preferred embodiment, a fusion polypeptide can be represented by the general formula X-subject polypeptide-Y, wherein the subject polypeptide represents a PGES polypeptide or analog or fragment thereof, and X and Y are independently absent or represent amino acid sequences which are not significantly related to a PGES sequence.

“Agent” refers to a chemical compound, a mixture of chemical compounds, a biological macromolecule (e.g., DNA, RNA, polypeptides or lipids), or an extract made from biological materials such as bacteria, plants, fungi, or animal (particularly mammalian) cells or tissues. Agents are evaluated, e.g., for potential activity as modulators of PGES by inclusion in screening assays and methods of identification described herein.

“Naturally-occurring” refers to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring.

The following terms are used to describe the sequence relationships between two or more polynucleotides: “reference sequence”, “comparison window”, “sequence identity”, “percentage of sequence identity”, and “substantial identity.” A “reference sequence” is a defined sequence used as a basis for a sequence comparison; a reference sequence may be a subset of a larger sequence, for example, as a segment of a full-length cDNA or gene sequence given in a sequence listing, such as a polynucleotide sequence of SEQ ID NO: 1, or may comprise a complete cDNA or gene sequence. Generally, a reference sequence is at least 20 nucleotides in length, frequently at least 25 nucleotides in length, and often at least 50 nucleotides in length. Since two polynucleotides may each (1) comprise a sequence (i.e., a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window”, as used herein, refers to a conceptual segment of at least 20 contiguous nucleotide positions wherein a polynucleotide sequence may be compared to a reference sequence of at least 20 contiguous nucleotides and wherein the portion of the polynucleotide sequence in the comparison window may comprise additions or deletions (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman (1981) Adv. Appl. Math. 2: 482, by the homology alignment algorithm of Needleman and Wunsch (1970) J. Mol. Biol. 48: 443, by the search for similarity method of Pearson and Lipman (1988) Proc. Natl. Acad. Sci. (U.S.A.) 85: 2444, by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of homology over the comparison window) generated by the various methods is selected. The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparision (i.e., the window size), and multiplying the result by 100 to yield the percentage of sequence identity. The terms “substantial identity” as used herein denotes a characteristic of a polynucleotide sequence, wherein the polynucleotide comprises a sequence that has at least 85 percent sequence identity, preferably at least 90 to 95 percent sequence identity, more usually at least 99 percent sequence identity as compared to a reference sequence over a comparison window of at least 20 nucleotide positions, frequently over a window of at least 25-50 nucleotides, wherein the percentage of sequence identity is calculated by comparing the reference sequence to the polynucleotide sequence which may include deletions or additions which total 20 percent or less of the reference sequence over the window of comparison. The reference sequence may be a subset of a larger sequence, for example, as a segment of the full-length human PGES polynucleotide sequence shown in SEQ ID NO: 1 or the full-length murine or bovine PGES cDNA sequence. An “unrelated” or “non-homologous” sequence shares less than 40% identity, though preferably less than 25% identity, with one of the PGES sequences of the present invention.

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity or more (e.g., 99 percent sequence identity). Preferably, residue positions which are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine.

“PGES fragment” as used herein refers to a polypeptide that has an amino-terminal and/or carboxy-terminal deletion, but where the remaining amino acid sequence is identical to the corresponding positions in the PGES sequence, e.g., SEQ ID NO: 2. PGES fragments typically are at least 14 amino acids long, preferably at least 20 amino acids long, usually at least 50, 60, 70, 80, 90, 100, 110, 120, 130, 140 or 150 amino acids long or longer.

“PGES analog” or “PGES variant” of a PGES polypeptide refers to naturally- or non-naturally-occurring polypeptides which have a certain homology to a PGES polypeptide, e.g., an amino acid sequence homology or a structural homology. Some PGES analogs may lack biological activity but may still be employed for various uses, such as for raising antibodies to PGES epitopes, as an immunological reagent to detect and/or purify PGES antibodies by affinity chromatography, or as a competitive or noncompetitive agonist, antagonist, or partial agonist of native PGES protein function.

PGES polypeptides and analogs thereof, as well as fragments of the PGES polypeptides or of the analogs, are referred to as “PGES-type polypeptides.”

A “PGES polypeptide” refers to a human polypeptide having SEQ ID NO: 2, naturally-occurring alleles thereof (including the sequence set forth in AF010316), paralogs, and to any non-human naturally-occurring polypeptide that is an ortholog of PGES (e.g., a non-human primate, a feline, a canine, an ovine or a bovine) or a paralog. Depending on the context, a “PGES polypeptide” can also refer to a composition comprising essentially only PGES polypeptides or preparations of PGES polypeptides.

“Cognate” or “ortholog” as used herein refers to a gene sequence or protein encoded thereby that is evolutionarily and functionally related between species. For example but not limitation, in the human genome, the human CD4 gene is the cognate gene to the mouse CD4 gene, since the sequences and structures of these two genes indicate that they are highly homologous and both genes encode a protein which functions in signaling T cell activation through MHC class II-restricted antigen recognition. Thus, the cognate murine gene to the human PGES gene is the murine gene which encodes an expressed protein which has the greatest degree of sequence identity to the human PGES protein and which exhibits an expression pattern similar to that of the human PGES(e.g., expressed in T lineage cells). Preferred cognate PGES genes are: rat PGES, rabbit PGES, canine PGES, nonhuman primate PGES, porcine PGES, bovine PGES, and hamster PGES.

As used herein, the terms “label” or “labeled” refers to incorporation of a detectable marker, e.g., by incorporation of a radiolabeled amino acid or nucleotide or attachment to a polypeptide or nucleic acid of biotinyl moieties that can be detected by marked avidin (e.g., streptavidin containing a fluorescent marker or enzymatic activity that can be detected by optical or colorimetric methods). Various methods of labeling polypeptides, nucleic acids, and glycoproteins are known in the art and may be used. Examples of labels for polypeptides include, but are not limited to, the following: radioisotopes (e.g., ³H, ¹⁴C, ³⁵s, ¹²⁵I, ¹³¹I), fluorescent labels (e.g., FITC, rhodamine, lanthanide phosphors), enzymatic labels (e.g., horseradish peroxidase, β-galactosidase, luciferase, alkaline phosphatase), biotinyl groups, predetermined polypeptide epitopes recognized by a secondary reporter (e.g., leucine zipper pair sequences, binding sites for secondary antibodies, metal binding domains, epitope tags). In some embodiments, labels are attached by spacer arms of various lengths to reduce potential steric hindrance.

A “pharmaceutical composition of an agent” comprises an agent and a suitable carrier, diluent or vehicle.

“Prostaglandin” and “PG” are generally used to describe a class of compounds which are analogues and derivatives of prostanoic acid (1). PG's may be further classified, for example, according to their 5-membered ring structure, using a letter designation; PGs of A-J series are known. PGs may be further classified based on the number of unsaturated bonds on the side chain, e.g., PG₁ s (13,14-unsaturated), PG₂ (13,14- and 5,6-unsaturated), and PG₃'s (13,14-,5,6- and 17,18-unsaturated). See U.S. Pat. No. 5,631,287.

“PGE” refers to prostaglandin E, e.g., PGE₁, PGE₂, PGE₃ and analogs thereof. PGE₁, PGE₂, PGE₃ are also referred to as PGE-1, PGE-2, PGE-3.

“PGH” refers to prostaglandin H, e.g., PGH₁, PGH₂, PGH₃ and analogs thereof.

“Substantially pure” refers to an object species is the predominant species present (i.e., on a molar basis it is more abundant than any other individual species in the composition), and preferably a substantially purified fraction is a composition wherein the object species comprises at least about 50 percent (on a molar basis) of all macromolecular species present. Generally, a substantially pure composition will comprise more than about 80 to 90 percent of all macromolecular species present in the composition. Most preferably, the object species is purified to essential homogeneity (contaminant species cannot be detected in the composition by conventional detection methods) wherein the composition consists essentially of a single macromolecular species.

“Isolated” as used herein with respect to nucleic acids, such as DNA or RNA, refers to molecules separated from other DNAs, or RNAs, respectively, that are present in the natural source of the macromolecule. For example, an isolated nucleic acid encoding one of the subject PGES polypeptides includes no more than the entire gene (including the promoter), usually no more than 10 kilobases (kb) of nucleic acid sequence which naturally immediately flanks the PGES gene in genomic DNA, preferably no more than 5 kb of such naturally occurring flanking sequences, and more preferably less than 1.5 kb of such naturally occurring flanking sequence. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides.

“Non-human animals” include mammals such as rodents, non-human primates, ovines, bovines, canines, felines, chickens, amphibians, reptiles, etc.

“Nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides.

“Promoter” means a DNA sequence that regulates expression of a selected DNA sequence operably linked to the promoter, and which effects expression of the selected DNA sequence in cells. The term encompasses constitutive and inducible (i.e. expression levels can be controlled) promoters.

“Protein,” “polypeptide” and “peptide” are used interchangeably herein when referring to a gene product.

“Recombinant protein” refers to a polypeptide that is produced by recombinant DNA techniques, wherein generally, DNA encoding a protein, e.g., a PGES polypeptide, is inserted into a suitable expression vector which is in turn used to transform a host cell to produce the protein.

“Small molecule” as used herein, is meant to refer to a composition, which has a molecular weight of less than about 5 kD and most preferably less than about 4 kD. Small molecules can be nucleic acids, peptides, polypeptides, peptidomimetics, carbohydrates, lipids or other organic (carbon containing) or inorganic molecules. Many pharmaceutical companies have extensive libraries of chemical and/or biological mixtures, often fungal, bacterial, or algal extracts, which can be screened with any of the assays of the invention, e.g., to identify compounds that modulate a PGES activity.

“Transcriptional regulatory sequence” is a generic term used throughout the specification to refer to DNA sequences, such as initiation signals, enhancers, and promoters, which induce or control transcription of protein coding sequences with which they are operably linked. In preferred embodiments, transcription of one of the subject genes is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally-occurring gene.

The term “subject polypeptide” or “subject protein” or “subject peptide” refers to a PGES polypeptide or analog or fragment thereof, which is a modulator, e.g., an agonist or antagonist, of a wild-type PGES polypeptide. The subject polypeptide may be a fusion polypeptide.

The term “subject nucleic acid” or “subject gene” refers to a nucleic acid or gene, respectively, encoding a subject PGES polypeptide.

The term “treating” as used herein is intended to encompass preventing, curing, and/or ameliorating at least one symptom of a condition or disease.

A nucleic acid encoding human PGES is described in Jakobsson et al. (1999) PNAS 96:7220 and in published PCT application WO 00/28022 by Jakobsson et al. The nucleotide sequence encoding the full length human PGES (as disclosed in Jakobsson et al., supra and in WO 00/28022) is set forth as SEQ ID NO: 1 and the amino acid sequence of human PGES is set forth as SEQ ID NO: 2. The nucleotide sequence encoding human PGES is also included in GenBank Accession Number R76492, which is the nucleotide sequence of expressed sequence tag clone 143735. GenBank Accession No. AF027740 consists of a portion of the sequence of R76492 including the coding sequence of human PGES (which corresponds to nucleotides 19-477 of AF027740). This sequence refers to the encoded protein as microsomal glutathione S-transferase 1-like 1 (MGST1-L1). Human PGES polypeptide consists of 152 amino acids (SEQ ID NO: 2) and is encoded by a nucleotide sequence consisting of 456 nucleotides (SEQ ID NO: 1).

A nucleic acid encoding human protein that differs from SEQ ID NO: 2 in one amino acid, i.e., the addition of a glycine between amino acids 53 and 54 was described in published PCT application WO 99/14356 by Vogelstein et al., in Polyak et al. (1997) Nature 389:300 and in GenBank Accession No. AF010316, all of which refer to the protein as p53-induced PIG12. The described protein of Vogelstein et al. may be an allelic or mutant form of human PGES or result from a sequencing error, and such a polypeptide is also included in the definition of PGES, and can be used according to the present invention.

A bovine PGES has been described in Filion et al., (2001) J. Biol. Chem., manuscript M103709200. A rat and a mouse PGES polypeptides have been described in Murakami et al., (2000) J. Biol. Chem. 275: 32783; Mancini et al. (2001) J. Biol. Chem. (in press); and Satoh et al. (2000) Neurosci. Lett. 283:221. The bovine polypeptide is more than 77% identical to human (SEQ ID NO: 2), mouse (Murakami et al., (2000) J. Biol. Chem. 275: 32783) and rat (Murakami et al., supra; Mancini et al. (2001) J. Biol. Chem. (in press); and Satoh et al. (2000) Neurosci. Lett. 283:221).

It is likely that the active site of PGES is located between about residues 30 to about 130. Indeed, the relevant catalytic region of the PGE synthase protein is expected to be in the central segment of SEQ ID NO: 2 based on analogy with MGST1 and LTC₄ synthase. Amino acids 1-41 can be removed from MGST1 without loss of function (Andersson et al. (1994) Biochem. Biophys. Acta 1204:298). C-terminal fragments can be exchanged between LTC₄ synthase and FLAp without alteration of protein function (Lam et al. (1997) J. Biol. Chem. 272:13923).

Furthermore, Murakami et al. (supra) have shown that Arg110 in human PGES is important for catalytic function. An alignment of the sequences of human, mouse, rat and bovine PGES (Filion et al., supra) indicates that this argining is conserved in all four species, further suggesting its role in catalytic activity. Also, two internal regions Arg39 to Ala51 and Asp63 to Pro97 of the bovine protein (i.e., Arg38 to Ala50 and Asp62 to Pro96, respectively, of SEQ ID NO: 2) show 100% identity across these four species, and therefore may contain important structural/regulatory domains. The N-terminal region (Met1 to Leu14) and carboxyl terminal region (Lys12l to Leu153) of the bovine protein (i.e., Met1 to Leu13 and Lys12O to Leu152 of SEQ ID NO: 2, respectively) appear much less conserved, with an average of 59 to 61% identity, respectively, when comparing the bovine PGES with the corresponding rodent enzymes (see Filion et al., supra).

Human PGES contains two hydrophilic loops, located at about amino acids 36 to 78 and 119 to 30 of SEQ ID NO: 2, respectively. These loops are most likely part of the active site of the enzyme. The region between the two loops, i.e., amino acids 79 to 118, contains hydrophobic regions and may also contain functional elements of the enzyme.

The invention provides purified polypeptides comprising a PGES polypeptide or analog or fragment thereof, e.g., fused to a heterologous polypeptide (referred to herein as “subject fusion polypeptide” or “PGES fusion polypeptide”). In a preferred embodiment, the PGES polypeptide or fusion polypeptide is substantially free of other cellular material, e.g., proteins. The term “substantially pure or purified preparations of a PGES polypeptide” refers to preparations of the PGES polypeptides having less than about 20% (by dry weight) contaminating cellular material, e.g., nucleic acids, proteins, and lipids, and preferably having less than about 5% contaminating cellular material. Preferred preparations of the subject fusion polypeptide have less than about 2% contaminating cellular material; even more preferably less than about 1% contaminating cellular material and most preferably less than about 0.5; 0.2; 0.1; 0.01; 0.001% contaminating cellular material.

Polypeptide preparations that are “substantially free of other cellular proteins” (also referred to herein as “contaminating proteins”) refer to preparations of polypeptides having less than about 20% (by dry weight) contaminating protein, and preferably having less than about 5% contaminating protein. Preferred preparations of the subject polypeptides have less than about 2% contaminating protein; even more preferably less than about 1% contaminating protein and most preferably less than about 0.5; 0.2; 0.1; 0.01; 0.001% contaminating proteins.

Those skilled in the art will appreciate that the purity of the polypeptide preparation of the invention can be determined by various methods. A preferred method for determining the amount of contaminating proteins in a polypeptide preparation comprises subjecting the polypeptide preparation to gel electrophoresis, e.g., polyacrylamide electrophoresis, in the presence of specific amounts of molecular markers, and staining the gel after the electrophoresis with a protein dye. A comparison of the intensity of the band of the subject polypeptide with the molecular markers indicates the purity of the subject polypeptide preparation. Other methods for determining the amount of contaminating proteins include mass spectrometry, gel filtration and peptide sequencing according to methods known in the art.

A preferred method for determining the amount of contaminating cellular material in a polypeptide preparation comprises gel electrophoresis and silver staining of the gel.

Other methods for determining the purity of a polypeptide preparation include mass spectrometry according to methods known in the art. Yet other measurements of the purity of a polypeptide preparation include a measure of the activity of the polypeptide, as further described herein.

Protein concentrations can be determined according to the following methods: Lowry-Folin-Ciocalteau reagent; UV absorption at 280 nm (aromatic band) or 205-220 nm (peptide band); dye binding (e.g., Coomassie Blue G-250); or bis-cinchonic acid (BCA; Pierce Chemicals (Rockford, Ill.)) reagent. All of these methods are described in, e.g., Robert K. Scopes, Protein Purification, Principles and Practice, Third Ed., Springer Verlag N.Y., 1993, and references cited therein. Briefly, the well-known Lowry method is a relatively sensitive method giving a good color with 0.1 mg/ml or protein or less. The method using Coomassie Blue G-250 is very sensitive, fast and at least as accurate as the Lowry method. The procedure consists in mixing a polypeptide sample with the reagent and measure the blue color at 595 nm.

Those skilled in the art will understand that the preferred method for determining exact protein amounts is by dry weight determination, since it provides a suitably accurate measurement of protein amount. Thus, in a preferred embodiment for determining the amount of the purified polypeptide of the invention, the dry weight of a highly pure preparation of the polypeptide of the invention is determined, and this preparation is then used as a standard for determining the protein concentration of other preparations of polypeptides of the invention.

As those skilled in the art will understand, the percent recovery and degree of purity of a preparation of polypeptide of the invention can be calculated from the total amount of protein recovered after purification and the amount and/or activity of the polypeptide of interest.

The subject fusion polypeptides comprise a PGES polypeptide or analog or fragment thereof and a polypeptide which is heterologous thereto.

As used herein, a PGES polypeptide refers to a human polypeptide having SEQ ID NO: 2, naturally-occurring alleles thereof, and to any non-human naturally-occurring polypeptide that is an ortholog of PGES (e.g., a non-human primate, a feline, a canine, an ovine or a bovine). Analogs or variants of a PGES polypeptide refers to naturally- or non-naturally-occurring polypeptides which have a certain homology to a PGES polypeptide, e.g., an amino acid sequence homology or a structural homology. For simplicity, PGES polypeptides and analogs thereof, as well as fragments of the PGES polypeptides or of the analogs, are referred to as “PGES-type polypeptides.” Accordingly, in a preferred embodiment, the invention provides fusion polypeptides comprising a PGES-type polypeptide and a polypeptide that is heterologous thereto.

Preferred PGES-type polypeptides of the invention have an amino acid sequence which are at least about 60%, 70%, 80%, 85%, 90%, or 95% identical or homologous to the amino acid sequence of SEQ ID NO: 2. Even more preferred PGES-type polypeptides comprise an amino acid sequence which is at least about 97, 98, or 99% identical or homologous to the amino acid sequence of SEQ ID NO: 2.

For example, a PGES-type polypeptide can be encoded by a nucleic acid, which is at least 85% homologous and more preferably 90% homologous and most preferably 95% homologous with a nucleotide sequence set forth in SEQ ID NO: 1. Polypeptides which are encoded by a nucleic acid that is at least about 98-99% homologous to SEQ ID NO: 1 are also within the scope of the invention.

Another aspect of the invention provides polypeptides which are encoded by nucleic acids which hybridize under stringent conditions to a nucleic acid represented by the complement of SEQ ID NO: 1. Appropriate stringency conditions which promote DNA hybridization, for example, 6.0×sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y. (1989), 6.3.1-6.3.6. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature of salt concentration may be held constant while the other variable is changed. In a preferred embodiment, a nucleic acid encoding a PGES-type polypeptide hybridizes to the complement of SEQ ID NO: 1 under moderately stringent conditions, for example at about 2.0×SSC and about 40° C. In a particularly preferred embodiment, a nucleic acid encoding a PGES-type polypeptide hybridizes to the complement of SEQ ID NO: 1 under highly stringent conditions, e.g., in 5×SSC at 65° C., followed by a wash in 0.2×SSC, 0.1% SDS at 65° C.

Other PGES-type polypeptides that can be used in the instant fusion polypeptides include PGES-type polypeptides that are evolutionarily related to the human PGES polypeptide having SEQ ID NO: 2. The term “evolutionarily related to”, with respect to amino acid sequences of PGES polypeptides, refers to both polypeptides having amino acid sequences which have arisen naturally, and also to mutational variants of human PGES polypeptides which are derived, for example, by combinatorial mutagenesis.

In a preferred embodiment, a PGES-type polypeptide is a vertebrate PGES-type polypeptide, e.g., a mammalian PGES-type polypeptide, such as a human, non-human primate, rodent (mouse or rat), feline, bovine, or ovine PGES-type polypeptide. In a particularly preferred embodiment the PGES-type polypeptide is a PGES polypeptide, e.g., a human PGES polypeptide having the amino acid sequence set forth as SEQ ID No: 2. A bovine PGES has been described in Filion et al., (2001) J. Biol. Chem., manuscript M103709200. A rat and a mouse PGES polypeptides have been described in Murakami et al., (2000) J. Biol. Chem. 275: 32783; Mancini et al. (2001) J. Biol. Chem. (in press); and Satoh et al. (2000) Neurosci. Lett. 283:221.

Also within the scope of the invention are fusion polypeptides comprising a PGES-type polypeptide which is a fragment of a PGES polypeptide or analog thereof. The fragments can correspond to one or more particular motifs and/or domains or to arbitrary sizes, for example, at least about 5, 10, 25, 50, 75, 100 and 125, amino acids in length. Preferred fragments are those which are biologically active, e.g., fragments including the catalytic site of a PGES polypeptide, or corresponding portion of an analog thereof. Accordingly, exemplary fragments of PGES polypeptide having SEQ ID NO: 2 or ortholog thereof include about amino acids 1 to about amino acids 130; about amino acids 30-152; about amino acids 30-130; about amino acids 50 to 100; about amino acids 38 to 50; and about amino acids 62 to 96. Biological activity of a polypeptide can be determined according to methods described herein, such as an assay for determining the ability of a polypeptide to catalyze the conversion of a PGH into a PGE.

Other preferred PGES-type polypeptides include one or more hydrophilic loops, e.g., from about amino acid 36 to about amino acid 78 and from about amino acid 119 to about amino acid 130 of SEQ ID NO: 2. Preferred PGES-type polypeptides include those comprising an active site or portion thereof, such as one or two hydrophilic loops, and optionally the sequence between the two loops.

PGES-type polypeptides are preferably capable of functioning in one of either role of an agonist or antagonist of at least one biological activity of a PGES polypeptide, such as human PGES having SEQ ID NO: 2. Accordingly, a PGES-type polypeptide can be an enzyme that is capable of catalyzing the conversion of a PGH or analog thereof into a PGE or analog thereof, e.g., the conversion of PGH₂ into PGE₂, e.g., in the presence of reduced glutathione. A PGES-type polypeptide can also be a polypeptide that inhibits the conversion of a PGH into a PGE. Assays for measuring a biological activity of a PGES polypeptide are further described herein.

Preferred agonists of a wild-type PGES polypeptide have a specific activity of from about 1 to about 500 μmol/min/mg measured at either 0° C., room temperature, or 37° C. for the glutathione dependent conversion of PGH₂ to PGE₂. Preferred agonists have a specific activity from about 10 to about 250 μmol/min/mg. The unit refers to μmoles of PGE₂ produced per minute reaction time per milligram of PGES polypeptide. Exemplary PGES polypeptides purified as described herein have an activity of 15 μmol/min/mg measured at 0° C. and of 167 μmol/min/mg measured at 37° C. Preferred agonists have a specific activity of at least about 10, 20, 50, 100, 200, 300 μmol/min/mg. The specific activity of a PGES polypeptide or analog or fragment thereof can be determined by methods described in the Examples, which are also well known in the art.

Preferred agonists of a wild-type PGES polypeptide have a Michaelis constant (Km) for PGH₂ of about 1 to about 500 μM measured at either 0° C., room temperature, or 37° C. for the glutathione dependent conversion of PGH₂ to PGE₂. Preferred agonists have a Km of about 10 to about 200 μM. Even more preferred agonists have a Km of 100 μM, 10 μM, 5 μM, 1 μM, 0.5 μM, 0.1 μM or less. Exemplary PGES polypeptides purified as described herein have a Km of about 50 at 0° C. and of about 163 at 37° C.

Preferred agonists of a wild-type PGES polypeptide have a maximum reaction velocity (Vmax) of about 0.01 to about 10 μM/sec measured at either 0° C., room temperature, or 37° C. for the glutathione dependent conversion of PGH₂ to PGE₂. Preferred agonists have a Vmax of about 0.1 to about 5 μM/sec. Even more preferred agonists have a Vmax of at least about 0.1, 0.5, 1, 1.5, 2, 2.5, or 5 μM/sec.

Preferred PGES agonists and antagonists preparations have a concentration of at about 0.1 to about 20 mg/ml, more preferably from about 0.5 to about 10 mg/ml, and preferably at least about 0.1, 0.2, 0.5, 0.6, 0.7, 0.8, 0.9, 1, 1.2, 1.5, 1.7, 2, 2.5, 5, 7, 10 mg/ml.

Variants and fragments of PGES polypeptides, e.g., modulators such as agonists and antagonists, are useful in order to promote or inhibit only a subset of the biological activities of the naturally-occurring form of the protein. Thus, specific biological effects can be elicited by treatment with homologs of specific function, and with possibly fewer side effects relative to treatment with modulators, e.g, agonists and antagonists, which homolog functions are directed to all of the biological activities.

As those skilled in the art will appreciate, variants of the subject PGES proteins can be generated using conventional techniques, such as mutagenesis, including creating discrete point mutation(s), or by truncation. For instance, mutation can give rise to variants which retain substantially the same, or merely a subset, of the biological activity of a PGES polypeptide from which it was derived. Alternatively, antagonistic forms of the protein can be generated which are able to inhibit the function of the naturally occurring form of the protein, such as by competitively binding to a substrate of PGES.

Variants of PGES polypeptides of the present invention also include homologs of the wild-type PGES polypeptides, such as versions of those polypeptides which are resistant to proteolytic cleavage, as for example, due to mutations which alter ubiquitination or other enzymatic targeting associated with the polypeptide. Such sites are known in the art.

PGES polypeptides may also be chemically modified to create PGES derivatives by forming covalent or aggregate conjugates with other chemical moieties, such as glycosyl groups, lipids, phosphate, acetyl groups and the like. Covalent derivatives of PGES polypeptides can be prepared by linking the chemical moieties to functional groups on amino acid sidechains of the protein or at the N-terminus or at the C-terminus of the polypeptide.

Modification of the structure of the subject PGES polypeptides can be for such purposes as enhancing therapeutic or prophylactic efficacy, stability (e.g., ex vivo shelf life and resistance to proteolytic degradation), or post-translational modifications (e.g., to alter phosphorylation pattern of protein). Such modified peptides, when designed to retain at least one activity of the naturally-occurring form of the protein, or to produce specific antagonists thereof, are considered functional equivalents of PGES polypeptides. Such modified peptides can be produced, for instance, by amino acid substitution, deletion, or addition. The substitutional variant may be a substituted conserved amino acid or a substituted non-conserved amino acid.

For example, it is reasonable to expect that an isolated replacement of a leucine with an isoleucine or valine, an aspartate with a glutamate, a threonine with a serine, or a similar replacement of an amino acid with a structurally related amino acid (i.e. isosteric and/or isoelectric mutations) will not have a major effect on the biological activity of the resulting molecule. Conservative replacements are those that take place within a family of amino acids that are related in their side chains. Genetically encoded amino acids can be divided into four families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. In similar fashion, the amino acid repertoire can be grouped as (1) acidic=aspartate, glutamate; (2) basic=lysine, arginine histidine, (3) aliphatic=glycine, alanine, valine, leucine, isoleucine, serine, threonine, with serine and threonine optionally be grouped separately as aliphatic-hydroxyl; (4) aromatic=phenylalanine, tyrosine, tryptophan; (5) amide=asparagine, glutamine; and (6) sulfur-containing=cysteine and methionine. (see, for example, Biochemistry, 2^{nd} ed., Ed. by L. Stryer, W H Freeman and Co.: 1981). Whether a change in the amino acid sequence of a peptide results in a functional PGES homolog (e.g., functional in the sense that the resulting polypeptide mimics or antagonizes the wild-type form) can be readily determined by assessing the ability of the variant peptide to effect a biological activity of PGES, such as to catalyze the conversion of a PGH into a PGE, in a fashion similar to the wild-type protein, or competitively inhibit such a biological activity. Polypeptides in which more than one replacement has taken place can readily be tested in the same manner.

This invention further provides methods for generating sets of combinatorial mutants of the subject PGES polypeptides as well as truncation mutants, and is especially useful for identifying potential variant sequences (e.g., homologs). A purpose of screening such combinatorial libraries is to generate, for example, novel PGES homologs which can act as modulators and/or possess novel activities all together. Thus, combinatorially-derived homologs can be generated to have an increased potency relative to a naturally occurring form of the protein.

In one embodiment, a variegated library of PGES variants is generated by combinatorial mutagenesis at the nucleic acid level, and is encoded by a variegated gene library. For instance, a mixture of synthetic oligonucleotides can be enzymatically ligated into gene sequences such that the degenerate set of potential PGES sequences are expressible as individual polypeptides, or alternatively, as a set of larger fusion proteins (e.g., for phage display) containing the set of PGES sequences therein.

There are many ways by which such libraries of potential PGES homologs can be generated from a degenerate oligonucleotide sequence. Chemical synthesis of a degenerate gene sequence can be carried out in an automatic DNA synthesizer, and the synthetic genes then ligated into an appropriate expression vector. The purpose of a degenerate set of genes is to provide, in one mixture, all of the sequences encoding the desired set of potential PGES sequences. The synthesis of degenerate oligonucleotides is well known in the art (see for example, Narang, SA (1983) Tetrahedron 39:3; Itakura et al. (1981) Recombinant DNA, Proc 3^{rd} Cleveland Sympos. Macromolecules, ed. A G Walton, Amsterdam: Elsevier pp 273-289; Itakura et al. (1984) Annu. Rev. Biochem. 53:323; Itakura et al. (1984) Science 198:1056; Ike et al. (1983) Nucleic Acid Res. 11:477. Such techniques have been employed in the directed evolution of other proteins (see, for example, Scott et al. (1990) Science 249:386-390; Roberts et al. (1992) PNAS 89:2429-2433; Devlin et al. (1990) Science 249: 404-406; Cwirla et al. (1990) PNAS 87: 6378-6382; as well as U.S. Pat. Nos. 5,223,409, 5,198,346, and 5,096,815).

Likewise, a library of coding sequence fragments can be provided for a PGES clone in order to generate a variegated population of PGES fragments for screening and subsequent selection of bioactive fragments. A variety of techniques are known in the art for generating such libraries, including chemical synthesis. In one embodiment, a library of coding sequence fragments can be generated by (i) treating a double stranded PCR fragment of a PGES coding sequence with a nuclease under conditions wherein nicking occurs only about once per molecule; (ii) denaturing the double stranded DNA; (iii) renaturing the DNA to form double stranded DNA which can include sense/antisense pairs from different nicked products; (iv) removing single stranded portions from reformed duplexes by treatment with S1 nuclease; and (v) ligating the resulting fragment library into an expression vector. By this exemplary method, an expression library can be derived which codes for N-terminal, C-terminal and internal fragments of various sizes.

A wide range of techniques are known in the art for screening gene products of combinatorial libraries made by point mutations or truncation, and for screening cDNA libraries for gene products having a certain property. Such techniques will be generally adaptable for rapid screening of the gene libraries generated by the combinatorial mutagenesis of PGES homologs. The most widely used techniques for screening large gene libraries typically comprises cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Each of the illustrative assays described below are amenable to high through-put analysis as necessary to screen large numbers of degenerate PGES sequences created by combinatorial mutagenesis techniques.

Combinatorial mutagenesis has a potential to generate very large libraries of mutant proteins, e.g., in the order of 1026 molecules. Combinatorial libraries of this size may be may be screened, e.g., by recrusive ensemble mutagenesis (REM), which allows one to avoid the very high proportion of non-functional proteins in a random library and simply enhances the frequency of functional proteins, thus decreasing the complexity required to achieve a useful sampling of sequence space. REM is an algorithm which enhances the frequency of functional mutants in a library when an appropriate selection or screening method is employed (Arkin and Yourvan, 1992, PNAS USA 89:7811-7815; Yourvan et al., 1992, Parallel Problem Solving from Nature, 2., In Maenner and Manderick, eds., Elsevir Publishing Co., Amsterdam, pp. 401-410; Delgrave et al., 1993, Protein Engineering 6(3):327-331).

The heterologous polypeptide that is fused to a PGES polypeptide, analog or fragment thereof, in the subject fusion polypeptides can be any polypeptide that allows affinity purification of the polypeptide attached thereto.

The heterologous polypeptide can be fused to the N-terminus or the C-terminus of a polypeptide. Alternatively, in certain embodiments, it may be appropriate to include the heterologous polypeptide within the PGES polypeptide, analog or fragment thereof, provided as the conformation of the heterologous polypeptide allows binding to an affinity matrix and preferably does not affect the desired biological activity, e.g., ability to catalyze the conversion of a PGH into a PGE, e.g, PGH₂ into PGE₂. Small scale purification tests can be conducted to determine whether purification of the fusion polypeptide is more efficient when the heterologous polypeptide is located at the N- or C-terminus of the PGES polypeptide, analog or fragment thereof.

In a preferred embodiment, the heterologous polypeptide comprises one or more histidine residues, to enable the fusion polypeptide to bind to a metal, e.g., Fe, Co, Ni, Cu, Zn, and Al. A preferred heterologous polyeptide binds to a Ni²⁺ metal resin. The heterologous polypeptides can contain 1, 2, 3, 4, 5, 6, 7, 8, 9, 10, 11, 12 or more histidine residues. In an even more preferred embodiment, the heterologous polypeptide comprises six histidine residues (“His₆ tag”). Certain expression vectors comprising a sequence encoding six or ten histidines are commercially available, and usually comprise one or more restriction sites C-terminal or N-terminal to the sequence encoding the histidines, such that a nucleic acid encoding a polypeptide of interest can be fused in frame thereto, and a fusion polypeptide is expressed. Such vectors are available, e.g., from Qiagen (Chatsworth, Calif.) (e.g., pQE-TriSystem Vector) and from Novagen (Madison, Wis.) (pET-vectors). Of course, any other vector can be modified to include a sequence encoding six histidines, using methods well known in the art, and as further described herein.

A His₆ polypeptide tag is uncharged at physiological pH. In addition, it rarely atlers or contributes to protein immunogenicity, and rarely interferes with protein structure or function, and does not interfere with protein secretion (Sisk et al. (1994) J. Virol. 68:766).

A preferred fusion polypeptide of the invention consists of the full length human PGES fused at its N-terminus to a polypeptide consisting of six histidine residues (see Examples). The amino acid sequence is set forth in SEQ ID NO: 4 and an exemplary nucleic acid encoding the polypeptide is set forth in SEQ ID NO: 3.

In another embodiment, a protease cleavage site sequence or an enterokinase cleavage site sequence can be inserted between the sequence encoding the PGES-type polypeptide and that encoding the heterologous polypeptide. The purification leader sequence can then be subsequently removed by treatment with the protease or enterokinase to provide the purified protein (e.g., see Hochuli et al. (1987) J. Chromatography 411:177; and Janknecht et al. PNAS 88:8972). An exemplary protease recognition site is a Factor Xa protease recognition site. A vector including a sequence encoding a stretch of six histidines and a site susceptible to a prolease is available, e.g., from Qiagen (Chatsworth, Calif.) (pQE-30 Xa Vector containing a Factor Xa Protease recognition site).

Another heterologous polypeptide that can be used includes at least a portion of glutathione-S-transferase (GST). GST fusion proteins can contribute to purification of the fusion polypeptide, as for example by the use of glutathione-derivatized matrices (see, for example, Current Protocols in Molecular Biology, eds. Ausubel et al. (N.Y.: John Wiley & Sons, 1991)). Yet other heterologous polypeptides that can be used for affinity purification is maltose binding protein and protein A. When using these heterologous polypeptides, it may be desirable to include a protease site allowing the removal of the heterolgous polypeptide after affinity purification, since these heterologous polypeptides are larger than a His₆ tag.

In addition to the PGES-type polypeptide and a polypeptide heterologous thereto, a fusion polypeptide of the invention can also comprise other polypeptides, e.g., polypeptides which provide enhanced stability and/or solubility to a polypeptide. For example, immunoglobulin portions can be added to the fusion polypeptide of the invention. PGES-Ig fusion proteins can be prepared as described e.g., in U.S. Pat. No. 5,434,131.

Yet other polypeptide components that can be added to a fusion polypeptide include tags that allow the detection of a polypeptide. Frequently used Tags include myc-epitopes (e.g., see Ellison et al. (1991) J. Biol Chem 266:21150-21157) which includes a 10-residue sequence from c-myc, the pFLAG system (International Biotechnologies, Inc.), the pEZZ-protein A system (Pharmacia, N.J.), and a 16 amino acid portion of the Haemophilus influenza hemagglutinin protein. Furthermore, any polypeptide can be used as a Tag so long as a reagent, e.g., an antibody interacting specifically with the Tag polypeptide is available or can be prepared or identified.

In another embodiment, a signal peptide sequence is added to the construct, such that the fusion polypeptide is secreted from cells. Such signal peptides are well known in the art.

As indicated in the examples set out below, human PGES polypeptide-encoding nucleic acids can be obtained by polymerase chain reaction (PCR), or other method of amplification, from a nucleic acid encoding a PGES polypeptide, e.g., the expressed sequence tag clone 14735. Alternatively, a nucleic acid encoding a PGES polypeptide can be obtained from mRNA present in any of a number of eukaryotic cells, e.g., from A549 and HeLa cells (see Jakobssen et al. (1999) PNAS 96:7220). The method may include the synthesis of a first cDNA strand from the mRNA, followed by PCR amplification using two primers which hybridize to a PGES nucleic acid, and isolation of the amplified product.

A nucleic acid encoding a PGES polypeptide of the invention can also be cloned from any suitable source, e.g., from a cDNA or a genomic library. cDNA encoding a PGES polypeptide or fragment thereof can be obtained by isolating total mRNA from a cell, e.g., a vertebrate cell, a mammalian cell, or a human cell. Double stranded cDNAs can then be prepared from the total mRNA, and subsequently inserted into a suitable plasmid or bacteriophage vector using any one of a number of known techniques.

Nucleic acids encoding non-human PGES can be obtained, e.g., by low-stringency hybridizations with a nucleic acid comprising all or part of SEQ ID NO: 1 or by amplification, e.g., PCR amplification using one or more degenerate primers. Nucleic acid sequences encoding mouse and rat PGES are provided in GenBank under Accession Nos. NP_{—}071860 and NP_{—}067594, respectively. Nucleic acids encoding variants and fragments of PGES polypeptides can be prepared by mutagenesis or PCR methods, both of which are well known in the art.

Nucleic acids encoding PGES polypeptides or analogs or fragments thereof for use in the invention may differ from the wild-type nucleotide sequences, e.g., shown in SEQ ID NO: 1 or complement thereof due to degeneracy in the genetic code. Such nucleic acids encode functionally equivalent polypeptides. For example, a number of amino acids are designated by more than one triplet. Codons that specify the same amino acid, or synonyms (for example, CAU and CAC each encode histidine) may result in “silent” mutations which do not affect the amino acid sequence of a PGES polypeptide.

The nucleic acids encoding PGES polypeptides or fragments thereof can also be allelic variants, e.g., allelic variants of SEQ ID NO: 1. Such allelic variants may encode the polypeptide in SEQ ID NO: 2 or a polypeptide differing therefrom in one or more amino acids.

Preferred variants of nucleic acids encoding a PGES-type polypeptide are those nucleic acids in which certain codons have been changed to codons that are highly used in the particular expression system used for expressing the polypeptide (“codon bias”). Such substitutions are known to improve the expression of polypeptides in particular systems.

Nucleic acids encoding a heterologous polypeptide allowing the purification of a PGES polypeptide, analog or fragment thereof, can be obtained in a similar manner as PGES nucleic acids. Nucleic acids encoding other polypeptides that may be included in a fusion polypeptide, e.g., tags for detection, can also be made according to methods known in the art. Since some of these nucleic acids are short, they can also conveniently be synthesized as oligonucleotides. In this case, two oligonucleotides that are complementary to each are synthesized, following which they are hybridized together and ligated to the nucleic acid encoding the polypeptide of interest. In certain embodiments, double stranded oligonucleotides are first subjected to restriction digestion prior to being ligated.

Techniques for making fusion genes are known to those skilled in the art. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, eds. Ausubel et al. John Wiley & Sons: 1992).

Fusion polypeptides of the invention can be made in cell or in a lysate, e.g., a lysate prepared from cells. The cells, referred to herein as host cells, can be prokaryotic or eukaryotic cells. In a preferred embodiment, the nucleic acid encoding the fusion polypeptide is operably linked to transcriptional control sequences, e.g., a promoter and an enhancer. Generally, such nucleic acids are also incorporated into a plasmid or an expression vector, which is then introduced into a host cell to allow expression of the fusion polypeptide. The type of transcriptional control sequences used will depend on the particular expression system used, e.g., whether the system is prokaryotic (e.g., bacterial) or eukaryotic (e.g., yeast, avian, insect or mammalian). Mammalian transcriptional control elements are described, e.g,. in Transcriptional regulatory sequences are described in Goeddel; Gene Expression Technology: Methods in Enzymology 185, Academic Press, San Diego, Calif. (1990). Expression systems and appropriate transcriptional control sequences are further described below.

Suitable vectors for the expression of PGES-type polypeptides in prokaryotic cells, such as E. coli, include plasmids of the types: pBR322-derived plasmids, pEMBL-derived plasmids, pEX-derived plasmids, pBTac-derived plasmids and pUC-derived plasmids.

In one embodiment, the powerful phage T5 promoter, that is recognized by E. coli RNA polymerase is used together with a lac operator repression module to provide tightly regulated, high level expression or recombinant proteins in E. coli. In this sytem, protein expression is blocked in the presence of high levels of lac repressor. Such vectors are available commercially, e.g., from Qiagen (Chatsworth, Calif.) (QIAexpress pQE vectors).

In another embodiment, the nucleic acid encoding a PGES polypeptide fusion polypeptide is operably linked to a bacterial promoter, e.g., the anaerobic E. coli, NirB promoter or the E. coli lipoprotein llp promoter, described, e.g., in Inouye et al. (1985) Nucl. Acids Res. 13:3101; Salmonella pagC promoter (Miller et al., supra), Shigella ent promoter (Schmitt and Payne, J. Bacteriol. 173:816 (1991)), the tet promoter on Tn10 (Miller et al., supra), or the ctx promoter of Vibrio cholera. Any other promoter can be used in the invention. The bacterial promoter can be a constitutive promoter or an inducible promoter. A preferred inducible promoter is a promoter which is inducible by iron or in iron-limiting conditions. In fact, some bacteria, e.g., intracellular organisms, are believed to encounter iron-limiting conditions in the host cytoplasm. Examples of iron-regulated promoters of FepA and TonB are known in the art and are described, e.g., in the following references: Headley, V. et al. (1997) Infection & Immunity 65:818; Ochsner, U. A. et al. (1995) Journal of Bacteriology 177:7194; Hunt, M. D. et al. (1994) Journal of Bacteriology 176:3944; Svinarich, D. M. and S. Palchaudhuri. (1992) Journal of Diarrhoeal Diseases Research 10:139; Prince, R. W. et al. (1991) Molecular Microbiology 5:2823; Goldberg, M. B. et al. (1990) Journal of Bacteriology 172:6863; de Lorenzo, V. et al. (1987) Journal of Bacteriology 169:2624; and Hantke, K. (1981) Molecular & General Genetics 182:288.

The nucleic acid encoding the PGES fusion polypeptide and the bacterial promoter to which it is operably linked are preferably in a vector or plasmid. As used herein, the term “vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/or expression of nucleic acids contained therein. Vectors capable of directing the expression of genes to which they are operatively linked are referred to herein as “expression vectors.” The term “plasmid” as used herein, refers generally to circular double stranded DNA loops which are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

A plasmid for practicing the invention preferably comprises sequences required for appropriate transcription of the nucleic acid in bacteria, e.g., a transcription termination signal. The vector can further comprise sequences encoding factors allowing for the selection of bacteria comprising the nucleic acid of interest, e.g., gene encoding a protein providing resistance to an antibiotic, sequences required for the amplification of the nucleic acid, e.g., a bacterial origin of replication.

In a preferred embodiment, the DNA is operably linked to a first promoter and the bacterium further comprises a second DNA encoding a first polymerase which is capable of mediating transcription from the first promoter, wherein the DNA encoding the first polymerase is operably linked to a second promoter. In a preferred embodiment, the second promoter is a bacterial promoter, such as those delineated above. In an even more preferred embodiment, the polymerase is a bacteriophage polymerase, e.g., SP6, T3, or T7 polymerase and the first promoter is a bacteriophage promoter, e.g., an SP6, T3, or T7 promoter, respectively. Plasmids comprising bacteriophage promoters and plasmids encoding bacteriophage polymerases can be obtained commercially, e.g., from Promega Corp.(Madison, Wis.) and InVitrogen (San Diego, Calif.), or can be obtained directly from the bacteriophage using standard recombinant DNA techniques (J. Sambrook, E. Fritsch, T. Maniatis, Molecular Cloning: A Laboratory Manual, Cold Spring Laboratory Press, 1989). Bacteriophage polymerases and promoters are further described, e.g., in the following references: Sagawa, H. et al. (1996) Gene 168:37; Cheng, X. et al. (1994) PNAS USA 91:4034; Dubendorff, J. W. and F. W. Studier (1991) Journal of Molecular Biology 219:45; Bujarski, J. J. and P. Kaesberg (1987) Nucleic Acids Research 15:1337; and Studier, F. W. et al. (1990) Methods in Enzymology 185:60). Such plasmids can further be modified according to the specific embodiment of the invention.

In another preferred embodiment, the bacterium further comprises a DNA encoding a second polymerase which is capable of mediating transcription from the second promoter, wherein the DNA encoding the second polymerase is operably linked to a third promoter. In a preferred embodiment, the third promoter is a bacterial promoter. However, more than two different polymerases and promoters could be introduced in a bacterium to obtain high levels of transcription. The use of one or more polymerase for mediating transcription in the bacterium can provide a significant increase in the amount of PGES polypeptide in the bacterium relative to a bacterium in which the DNA is directly under the control of a bacterial promoter. The selection of the system to adopt will vary depending on the specific use of the invention, e.g., on the amount of RNA that one desires to produce.

Generally, a nucleic acid encoding a fusion polypeptide of the invention is introduced into a host cell, such as by transfection, and the host cell is cultured under conditions allowing expression of the fusion polypeptide. Methods of introducing nucleic acids into prokaryotic and eukaryotic cells are well known in the art. Suitable media for mammalian and prokaryotic host cell culture are well known in the art. Generally, the nucleic acid encoding the subject fusion polypeptide is under the control of an inducible promoter, which is induced once the host cells containing the nucleic acid have divided a certain number of times. For example, where a nucleic acid is under the control of a beta-galactose operator and repressor, isopropyl beta-D-thiogalactopyranoside (IPTG) is added to the culture when the bacterial host cells have attained a density of about OD₆₀₀ 0.45-0.60. The culture is then grown for some more time to give the host cell the time to synthesize the polypeptide. Cultures are then typically frozen and may be stored frozen for some time, prior to isolation and purification of the polypeptide.

When using a prokaryotic host cell, the host cell may include a plasmid which expresses an internal T7 lysozyme, e.g., expressed from plasmid pLysSL (see Examples). Lysis of such host cells liberates the lysozyme which then degrades the bacterial membrane.

Other sequences that may be included in a vector for expression in bacterial or other prokaryotic cells include a synthetic ribosomal binding site; strong transcriptional terminators, e.g., t₀ from phage lambda and t₄ from the rrnB operon in E. coli, to prevent read through transcription and ensure stability of the expressed polypeptide; an origin of replication, e.g., Co1E1; and beta-lactamase gene, conferring ampicilin resistance.

Other host cells include prokaryotic host cells. Even more preferred host cells are bacteria, e.g., E. coli. Other bacteria that can be used include Shigella spp., Salmonella spp., Listeria spp., Rickettsia spp., Yersinia spp., Escherichia spp., Klebsiella spp., Bordetella spp., Neisseria spp., Aeromonas spp., Franciesella spp., Corynebacterium spp., Citrobacter spp., Chlamydia spp., Hemophilus spp., Brucella spp., Mycobacterium spp., Legionella spp., Rhodococcus spp., Pseudomonas spp., Helicobacter spp., Vibrio spp., Bacillus spp., and Erysipelothrix spp. Most of these bacteria can be obtained from the American Type Culture Collection (ATCC; 10801 University Blvd., Manassas, Va. 20110-2209).

In a preferred embodiment, a fusion polypeptide, e.g., His₆-PGES (e.g., having SEQ ID NO: 4), is expressed in E. coli, e.g., BL21 (DE3). The E. coli strain optionally comprises plasmid pLysSL. An exemplary plasmid including a nucleotide sequence encoding His₆-PGES having SEQ ID NO: 4 is shown in FIG. 2B.

A number of vectors exist for the expression of recombinant proteins in yeast. For instance, YEP24, YIP5, YEP51, YEP52, pYES2, and YRP17 are cloning and expression vehicles useful in the introduction of genetic constructs into S. cerevisiae (see, for example, Broach et al. (1983) in Experimental Manipulation of Gene Expression, ed. M. Inouye Academic Press, p. 83, incorporated by reference herein). These vectors can replicate in E. coli due the presence of the pBR322 ori, and in S. cerevisiae due to the replication determinant of the yeast 2 micron plasmid. In addition, drug resistance markers such as ampicillin can be used.

Preferred mammalian expression vectors contain both prokaryotic sequences, to facilitate the propagation of the vector in bacteria, and one or more eukaryotic transcription units that are expressed in eukaryotic cells. The pcDNAT/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papillomavirus (BPV-1), or Epstein-Barr virus (pHEBo, pREP-derived and p205) can be used for transient expression of proteins in eukaryotic cells. The various methods employed in the preparation of the plasmids and transformation of host organisms are well known in the art. For other suitable expression systems for both prokaryotic and eukaryotic cells, as well as general recombinant procedures, see Molecular Cloning A Laboratory Manual, 2^{nd} Ed., ed. by Sambrook, Fritsch and Maniatis (Cold Spring Harbor Laboratory Press: 1989) Chapters 16 and 17.

In some instances, it may be desirable to express the subject fusion polypeptides by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the β-gal containing pBlueBac III).

The fusion polypeptide can also be produced in an in vitro system, e.g., in a in vitro translation system, e.g., cell lysate, e.g., a reticulocyte lysate. The term “in vitro translation system”, which is used herein interchangeably with the term “cell-free translation system” refers to a translation system which is a cell-free extract containing at least the minimum elements necessary for translation of an RNA molecule into a protein. An in vitro translation system Met typically comprises at least ribosomes, ^{t}RNAs, initiator methionyl-^{t}RNA, proteins or complexes involved in translation, e.g., eIF₂, eIF₃, the cap-binding (CB) complex, comprising the cap-binding protein (CBP) and eukaryotic initiation factor 4F (eIF₄F). A variety of in vitro translation systems are well known in the art and include commercially available kits. Examples of in vitro translation systems include eukaryotic lysates, such as rabbit reticulocyte lysates, rabbit oocyte lysates, human cell lysates, insect cell lysates and wheat germ extracts. Lysates are commercially available from manufacturers such as Promega Corp., Madison, Wis.; Stratagene, La Jolla, Calif.; Amersham, Arlington Heights, Ill.; and GIBCO/BRL, Grand Island, N.Y. In vitro translation systems typically comprise macromolecules, such as enzymes, translation, initiation and elongation factors, chemical reagents, and ribosomes.

In cases where plant expression vectors are used, the expression of a polypeptide of the invention may be driven by any of a number of promoters. For example, viral promoters such as the 35S RNA and 19S RNA promoters of CaMV (Brisson et al., 1984, Nature, 310:511-514), or the coat protein promoter of TMV (Takamatsu et al., 1987, EMBO J., 6:307-311) may be used; alternatively, plant promoters such as the small subunit of RUBISCO (Coruzzi et al., 1994, EMBO J., 3:1671-1680; Broglie et al., 1984, Science, 224:838-843); or heat shock promoters, eg., soybean hsp 17.5-E or hsp 17.3-B (Gurley et al., 1986, Mol. Cell. Biol., 6:559-565) may be used. These constructs can be introduced into plant cells using Ti plasmids, Ri plasmids, plant virus vectors; direct DNA transformation; microinjection, electroporation, etc. For reviews of such techniques see, for example, Weissbach & Weissbach, 1988, Methods for Plant Molecular Biology, Academic Press, New York, Section VIII, pp. 421-463; and Grierson & Corey, 1988, Plant Molecular Biology, 2d Ed., Blackie, London, Ch. 7-9.

An alternative expression system which can be used to express a polypeptide of the invention is an insect system. In one such system, Autographa californica nuclear polyhedrosis virus (AcNPV) is used as a vector to express foreign genes. The virus grows in Spodoptera frugiperda cells. The PGHS-2 sequence may be cloned into non-essential regions (for example the polyhedrin gene) of the virus and placed under control of an AcNPV promoter (for example the polyhedrin promoter). Successful insertion of the coding sequence will result in inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted gene is expressed. (e.g., see Smith et al., 1983, J. Virol., 46:584, Smith, U.S. Pat. No. 4,215,051).

In a specific embodiment of an insect system, the DNA encoding the subject polypeptide is cloned into the pBlueBacIII recombinant transfer vector (Invitrogen, San Diego, Calif.) downstream of the polyhedrin promoter and transfected into Sf9 insect cells (derived from Spodoptera frugiperda ovarian cells, available from Invitrogen, San Diego, Calif.) to generate recombinant virus. After plaque purification of the recombinant virus high-titer viral stocks are prepared that in turn would be used to infect Sf9 or High Five™ (BTI-TN-5B1-4 cells derived from Trichoplusia ni egg cell homogenates; available from Invitrogen, San Diego, Calif.) insect cells, to produce large quantities of appropriately post-translationally modified subject polypeptide. Although it is possible that these cells themselves could be directly useful for drug assays, the subject polypeptides prepared by this method can be used for in vitro assays.

In another embodiment, the subject polypeptides are prepared in transgenic animals, such that in certain embodiments, the polypeptide is secreted, e.g., in the milk of a female animal.

The fusion polypeptides of the invention, which are expressed in an expression system, e.g., as described above, are isolated from the cell culture medium or the host cells or both. For example, when the host cell is a prokaryotic host cell, e.g., an E. coli host cell, the recombinant polypeptide expressed in the host cell can be isolated from the culture medium if the protein is secreted or from the E. coli host cells or a particular compartment thereof, if the protein is not secreted. If a recombinant protein is attached to the bacterial membrane, it can be purified by first isolating the bacterial membranes. Alternatively, it can be isolated from whole cell lysates.

A preferred method of the invention for isolating recombinant polypeptides from membranes comprises first preparing a membrane lysate. For this, bacterial cells can freeze-thawed; the membranes lysed with lysozyme; and the DNA and RNA can be degraded. The lysate can then be homogenized by sonication at on ice. Debris and unbroken cells can be removed by low-speed centrifugation. Membrane fractions can then be pelleted by ultracentrifugation, followed by suspension of the pellet in buffer, e.g., sodium phosphate buffer. The membrane fraction can be frozen at this point. A recombinant polypeptide present in the membrane fraction can be isolated by solubilizing the membrane proteins in buffer containing a detergent, e.g., Triton X-100, at a temperature varying from zero to about 10° C., peferably on ice or at 4° C. Insoluble material can be separated by ultracentrifugation. This solubilized lysate can then be filtered through a filter, and the protein further purified as described below. Further details on these procedures are provided in the Examples.

Purification of a subject fusion polypeptide from a whole cell prokaryotic lysate, i.e., without prior membrane separation, can be performed as follows. Bacterial cells can be resuspended in a sodium phosphate buffer. The cells can then be lysed, e.g., with lysozyme, and the viscous cell lysate can be sonicated until obtaining a homogeneous mixture. A detergent, such as Triton X-100 can then be added to help solubilization, and ultracentrifugation can be used to remove insoluble material. This solubilized lysate is then ready for further purification described below. Further details on these procedures are provided in the Examples.

Numerous techniques are well known in the art for purifying proteins and any suitable technique can be used. These techniques include ion-exchange chromatography, gel filtration chromatography, ultrafiltration, electrophoresis, and immunoaffinity purification with antibodies specific for such peptide.

A preferred method of purification of the subject polypeptides include an affinity chromatography, such as a Ni affinity chromatography, preceded by a first step of purification. In a preferred embodiment, the first step of purification is a hydroxyapatite chromatography. It has been demonstrated herein that passing a solubilized lysate containing a GPES fusion polypeptide over an affinity chromatography was inefficient at removing a significant amount of impurities, and that an additional purification step prior to the affinity chromatography, e.g., hydroxyapatite chromatography, is necessary to obtain a homogeneously purified preparation of PGES, that can be used, e.g., in crystallographic studies.

Generally, any inorganic adsorbent can be used for the first chromatographic step. Inorganic substances that can be used for protein adsorption, include oxides, insoluble hydroxides, and phosphates. Examples of inorganic materials that have been used for protein purification include Alumina gel Cy (gel and crystalline), Bentonite (a silicaceous powder), Titanium oxide (TiO₂), Zinc hydroxide gel, and calcium phosphate in the form of Aged gel, Brushite or Hydroxyapatite. Inorganic materials are particularly useful for industrial applications, since they generally are cheaper than other types of chromatographic material.

A polypeptide binds to inorganic materials at least through electrostatic interaction. The interaction could also be a polar dipole-dipole bonding.

In a preferred embodiment, the inorganic substance is calcium hydroxyphosphate, which in crystalline form is known as hydroxyapatite. Batch adsorption can be carried out with calcium phosphate gel, which is a gelatinous form of hydroxyapatite. In another embodiment, the polypeptide is purified using another inorganic substance, either before or after the hydroxyapatite purification. For example, a preparation comprising the polypeptide of the invention is incubated with hydroxyapatite, the polypeptide is recovered in the unbound solution, which is then incubated with another inorganic substance, e.g., Bentonite.

Hydroxyapatite can be obtained commercially from several companies, e.g., Bio-Rad. Alternatively, it can be prepared, e.g., by slowly mixing together 0.5M solutions of CaCl₂ and Na₂HPO₄ with a solution of 1M NaCl, which results in the production of brushite, CaHPO₄₀.2H₂O. Boiling of the brushite with NaOH, converts it to hydroxyapatite, Ca₁₀(PO₄)₆(OH)₂ (Bernardi et al. (1971) Methods Enzymol. 22:325).

Inorganic materials for removing unwanted material from a polypeptide solution can be in the form of a batch or in a column-type system. In a preferred embodiment, a polypeptide solution (or preparation) is incubated together with an inorganic solvent in a batch-type system. After a short incubation, preferably on ice, the molecules that are not binding to the inorganic substance are recovered by separating out the inorganic substance from the solution. This can be done, e.g., by centrifugation to pellet the inorganic substance. In other embodiments, the polypeptide of interest may also adsorb to the inorganic substance under certain salt conditions, and can be recovered by incubation of the inorganic substance with higher salt concentrations.

In a preferred embodiment, adsorption is carried out in a pH range of about pH 6 and 9, and even more preferably at pH 8. Adsorption is preferably carried out in low salt concentration, but sufficient buffering should be present to avoid undesirable local pH changes in the highly polar environment of the adsorbent surface. A preferred buffer for using during adsorption is a phosphate buffer. A preferred method for purifying a polypeptide of the invention using hydroxyapatite is set forth in Example 4. It is expected that polypeptides having a similar local of global charge distribution relative to His₆hPGES described in Example 4, will not bind to hydroxyapatite in similar conditions to those described in Example 4, and can be recovered in the non-binding fraction.

Incubation of a polypeptide solution with inorganic substances allows elimination of a variety of undesirable polypeptides, other macromolecules, as well as low molecular weight compounds, which get adsorbed onto the inorganic substance, whereas the polypeptide of interest is not adsorbed. Further information regarding purification of polypeptides using inorganic substances is provided, e.g., in Robert K. Scopes, Protein Purification, Principles and Practice, Third Ed., Springer Verlag New York, 1993.

In a preferred embodiment, the second step of the purification of the polypeptide of the invention is a metal affinity chromatography, e.g., immobilized metal affinity chromatography (IMAC). This chromatography relies on the formation of weak coordinate bonds between metal ions immobilized on a column and basic groups on proteins, mainly histidine residues. The adsorbent can be formed by attaching to the matrix a suitable spacer arm plus a simple metal chelator, usually based on imino diaetate structures, e.g., in the form of EDTA (see Robert K. Scopes, supra). Imino diacetate (IDS) and tris(carboxymethyl) ethylene diamine (TED) can be used. These chelating ligands will bind tightly to metal ions, in particular to the divalent ions of the transition metals Fe, Co, Ni, Cu, and Zn, but also trivalent metal ions (Fe and Al).

Although histidine residues on proteins are most attracted to immobilized metals, other potential electron donating side chains include tryptophan and cysteine (at neutral pH). Binding to an affinity column requires that these amino acids are located on the surface of a polypeptide and the strength of interaction depends on the number of such linkages for the size of the polypeptide.

Metal chelate adsorbents are generally provided without the metal. Accordingly, in a first step, the column is loaded with the metal. In one embodiment, a solution containing, e.g., 50 mM of the metal salt, e.g., CuSO₄, Zn acetate or NiCl₂, is passed through the column until it is saturated with the metal. Excess metal ions are then washed out. This is preferably followed by a wash with a weak complexing agent, such as 1-10 mM imidazole or 0.5M glycine. In an even more preferred embodiment, the column is further washed with a more concentration solution of the weak complexing agent, e.g., from about 10 to about 100 mM imidazole, preferably from about 20 to about 80 mM imidazole, and even more preferably about 60 mM imidazole. It may also be desirable to wash the column with the solution that will be used for eluting the polypeptide of interest from the column to avoid having metal ions leach out into the final preparation.

IMAC is generally performed at high ionic strength, e.g., in 100 mM-1M NaCl, e.g., to avoid ion exchange effects. Application buffers generally are at pH 6 to 8, not containing complexing agents. The polypeptide solution is slowly passed through the column, generally at 4° C., e.g., at a flow rate of about 1 ml/minute. The column is then washed with start buffer until all unbound proteins are eluted. Elution of the polypeptide of interest can be achieved by either of two method. According to one method, the poypeptide is displayed by a stronger complexing agent, e.g., imidazole or EDTA. In the other method, the polypeptide is eluted by lowering the pH of the buffer, such that, e.g., the histidines on the polypeptide become protonated, and so are unable to coordinate with the metal ions. In certain embodiments, a combination of methods is used for eluting the polypeptide of the invention from the metal affinity matrix.

Accordingly, in a preferred embodiment, a solubilized lysate containing a fusion polypeptide comprising a PGES polypeptide, or analog or fragment thereof, fused to six histidines, is mixed with hydroxyapatite. The solution can also comprise Triton X-100. After a short incubation, preferably on ice, the hydroxyapatite is pelleted, such as by centrifugation, and the unbound fraction is removed and can be further cleared by centrifugation and filtration. This composition can then be loaded onto a chelating sepharose column charged with NiCl₂. After loading, the column is washed to remove unbound proteins. Another wash including 60 mM imidazole can be made to wash out unspecifically bound proteins. The histidine tagged polypeptide can then be eluted by the addition of about 200 to 400 mM imidazole, preferably about 300-350 mM imidazole. The preferred amount of imidazole may depend on the particular fusion polypeptide that is purified. For example, a fusion polypeptide consisting of the full length human PGES fused at its N-terminus to six histidines is efficiently eluted from the column with 350 mM imidazole.

The imidazole can then be removed from the solution containing the recombinant protein by dialysis or by passing it over a desalting column (see Examples). The preparation of the recombinant protein can further be purified by repeating a Ni affinity chromatography. This second Ni affinity chromatography can be run as the first one. Yet further rounds of purification can be undertaken.

Generally, sodium phosphate buffer can be substituted for potassium phosphate buffer. Other possible buffers include HEPES and Tris-HCl.

A PGES polypeptide without an affinity tag may be purified by hydroxyapatite followed by ion exchange chromatography.

The amount of protein that is eluted from a chromatography column can be determined, e.g., by the Coomassie protein assay according to the manufacturer's instructions (Bio-Rad).

In a preferred embodiment of the invention, the solubilization of proteins including PGES polypeptides (e.g., cell membranes or whole cell lysates) or purification of PGES polypeptides, analogs and fragments thereof, is performed in the presence of glycerol and/or reduced glutathione. As described in the Examples, the presence of glycerol and reduced glutathione (GSH) preserves the activity of solubilized PGES. Accordingly, it is preferable to include from 0.5 to 10 mM reduced glutathione; more preferable to include from about 1 to about 5 mM reduced glutathione; and most preferable to include from about 1 to about 2.5 mM reduced glutathione, in any solution comprising PGES. It is also preferable to include from about 1 to about 30% glycerol; more preferable to include from about 5 to about 15% glycerol; and even more preferable to include about 10% glycerol in any solution containing PGES.

Thus, glycerol and/or reduced glutathione are preferably included in any solution containing PGES, analog or fragments thereof, when it is desired to preserve the biological activity of the enzyme. Where, however, the presence of glycerol or GSH interferes with a particular step of the method used, it may be left out, and added immediately after the step has been performed.

The amount of proteins in a sample can be determined by the Coomassie protein assay according to the manufacturer's instructions (Bio-Rad). The amount and purity of proteins can also be determined by subjecting the protein mixtures to SDS-PAGE, optionally followed by Western blot analysis. SDS-PAGE gels can be stained, e.g., silver or Coomassie blue stained, for visualizing polypeptides. Western blots can also be incubated with a reagent binding specifically to the subject polylpeptide, e.g., an antibody. The inclusion of known amounts of reference proteins permit, by comparison, to estimate the quantity of a particular protein on the Western blot. Protocols for Western blot analysis are provided in the Examples and in Jakobssen et al., supra.

PGE synthase activity of the purified polypeptide, i.e., the ability to catalyze the conversion of a substrate of PGES into a 9-keto, 11-alpha-hydroxy form, e.g., PGH₂ into PGE₂ can be determined as follows. In one embodiment, the assay consists in incubating together a precursor of PGE₂, a reducing agent, preferably reduced glutathione and the PGE synthase. The polypeptide suspected of having PGES activity is incubated in a buffer, e.g., a sodium phosphate buffer in the presence of about 1 to 5 mM, preferably, 2.5 mM glutathione (GSH). The reaction is started by the addition of PGH₂ at about 5 to 20 μM, and preferably 10 uM, and the reaction mixture is then incubated either at 37° C., at room temperature, or preferably, at 0° C., for about one to ten minutes, and preferably, for about one minute. The reaction can be terminated by the addition of a solution reducing the pH to about 3 or 3.2. This can be achieved by the addition of acetonitrile/HCl (as described in Jakobsson et al. (1999) PNAS 96:7220) or by the addition of FeCl₂, citric acid, and/or 11-beta PGE₂, as described in the Examples. The formation of PGF₂ alpha, PGE₂ and PGD₂ can be determined by reverse-phase high pressure liquid chromatograph (HPLC) and UV detection at 195 nm. For subjecting the proteins to HPLC, the reaction mixtures can be subjected to solid phase extraction, e.g., using C₁₈ Chomabond columns; the samples eluted with acetone, dried and dissolved in acetonitrile. The samples are then ready for HPLC. More details of these methods are provided in the Examples, as well as in Jakobssen et al., supra, and in published PCT application No. WO 00/28022 by Jakobsson et al.

In embodiments in which the fusion polypeptide comprises a site that is recognized by a protease or enterokinase, e.g., for cleaving the heterologous polypeptide from the PGES polypeptide, treatment with the protease or enterokinase can be done at any stage of the purification of the peptide. It is preferably done prior to the last stage, however, since an additional step would then be necessary to remove the protease or enterokinase.

In certain assays, it may be desirable to include a labeled precursor, e.g., ³H PGH, such that PGE formed can be detected by the presence of the label, e.g., by using an online beta-RAM detector (Inus System, Tampa, Fla.).

In cases in which the subject fusion polypeptides are not produced in prokaryotes, the methods for purifying the subject fusion polypeptides can be adapted from any suitable methods based on the present description. Generally, a lysate is prepared from the host cell expressing the subject fusion polypeptide, and after some preliminary purification steps, e.g., centrifugations, the lysate is subjected to hydroxyapatite chromatography and then affinity chromatography, e.g., Ni affinity chromatography, as described herein. Similarly, where the subject fusion polypeptide is expressed in a lysate, e.g., a reticulocyte lysate, the fusion polypeptide can be purified as described herein.

In one embodiment, a purified PGES or analog thereof or fragment thereof is used to produce in vitro large quantities of any molecule whose synthesis includes isomerization by PGES. PGES catalyzes the conversion of a cyclic endoperoxide substrate into 9-keto, 11-alpha-hydroxy form of the substrate. PGES catalyzes the stereospecific formation of the core 9-keto, 11-alpha-hydroxy prostaglandin structure that is shared by numerous prostaglandin E molecules, e.g., PGE₁, PGE₂ and PGE₃. Accordingly, the invention provides methods for synthesizing PGE molecules and analogs thereof which comprise the core 9-keto, 111-alpha-hydroxy group.

Prostaglandin E molecules, e.g., PGE₂, can be prepared, e.g., by a scaled up version of the assay described above for testing the ability of a PGES polypeptide to catalyze the conversion of PGH₂ into PGE₂. Large amounts of precursors of PGEs, e.g., PGH₂, and reduced glutathione can be obtained commercially, e.g., from Cayman Chemical Company (Ann Arbor, Mich.) and Biomol Research Laboratories, Inc. (Plymouth Meeting, Pa.). Alternatively, PGH₂ substrate can be provided by incubation of cyclooxygenase and arachidonic acid. The prostaglandin E produced can then be purified according to methods known in the art including, e.g., separation on SP-HPLC with detection at 190-210 nm or separation by thin layer chromatography (TLC) using radioactive tracers as markers.

The invention provides PGES polypeptides, analogs and fragments thereof, of a high purity, which may be in the form of a fusion polypeptide or not (e.g., if the heterologous polypeptide has been cleaved off). These polypeptides can be used in vitro for identifying molecules binding to them and/or modulate an interaction between the polypeptide and its substrate, e.g., PGH₂. These molecules may act as inhibitors or stimulators of PGES activity. Such agents can be used for therapeutic purposes as further described herein.

Thus, the invention provides screening methods for identifying agonist and antagonist compounds of naturally-occurring PGES polypeptide, comprising selecting compounds which are capable of interacting with a PGES polypeptide or with a molecule interacting with a PGES polypeptide, such as a substrate of a PGES polypeptide, and/or selecting compounds which are capable of modulating the interaction of a PGES polypeptide with another molecule, such as a substrate peptide. A molecule which is capable of interacting with a PGES polypeptide is referred to herein as “PGES-binding partner” and can be a substrate, e.g., PGH₁, PGH₂,PGH₃ or an analog thereof or a portion thereof, so long as the analog or portion of the target peptide is capable of binding to a PGES polypeptide. A PGES-binding partner can also be a polypeptide which is not a substrate and which may, e.g., interact with a PGES polypeptide at a site other than the catalytic site.

The compounds of the invention can be identified using various assays depending on the type of compound and activity of the compound that is desired. Set forth below are at least some assays that can be used for identifying PGES therapeutics, i.e., molecules which affect the activity of a naturally-occurring PGES polypeptide. It is within the skill of the art to design additional assays for identifying PGES therapeutics.

In a preferred embodiment, cell-free assays for identifying PGES therapeutics consist essentially in a reaction mixture containing a subject polypeptide (e.g., a PGES polypeptide or analog or fragment thereof optionally fused to a heterologous polypeptide) and a test compound or a library of test compounds in the presence or absence of a binding partner. A preferred binding partner is PGH₂ or portions thereof sufficient for interacting with PGES. A test compound can be, e.g., a derivative of a PGES-binding partner, e.g., a biologically inactive substrate, or a small molecule.

Accordingly, one exemplary screening assay of the present invention includes the steps of contacting a subject polypeptide or functional fragment thereof or a PGES-binding partner with a test compound or library of test compounds and detecting the formation of complexes. For detection purposes, the molecule can be labeled with a specific marker and the test compound or library of test compounds labeled with a different marker. Interaction of a test compound with a subject polypeptide or fragment thereof or PGES-binding partner can then be detected by determining the level of the two labels after an incubation step and a washing step. The presence of two labels after the washing step is indicative of an interaction.

An interaction between molecules can also be identified by using real-time BIA (Biomolecular Interaction Analysis, Pharmacia Biosensor AB) which detects surface plasmon resonance (SPR), an optical phenomenon. Detection depends on changes in the mass concentration of macromolecules at the biospecific interface, and does not require any labeling of interactants. In one embodiment, a library of test compounds can be immobilized on a sensor surface, e.g., which forms one wall of a micro-flow cell. A solution containing the subject polypeptide or PGES-binding partner is then flown continuously over the sensor surface. A change in the resonance angle as shown on a signal recording, indicates that an interaction has occurred. This technique is further described, e.g., in BIAtechnology Handbook by Pharmacia.

Another exemplary screening assay of the present invention includes the steps of (a) forming a reaction mixture including: (i) a subject polypeptide, (ii) a PGES-binding partner (e.g., PGH₂), and (iii) a test compound; and (b) detecting interaction of the subject polypeptide and the PGES-binding protein. A statistically significant change (e.g., potentiation or inhibition) in the interaction of the subject polypeptide, analog or fragment thereof and PGES-binding protein in the presence of the test compound, relative to the interaction in the absence of the test compound, indicates that the test compound is a potential modulator, e.g., an agonist (mimetic or potentiator) or antagonist (inhibitor) of PGES activity. A “statistically significant change” refers to a difference of at least 50% in the affinity constant, preferably at least a factor of 2, even more preferably at least a factor of 5, and even more preferably at least a factor of 10.

Any suitable contacting of the compounds can be employed in the methods of the present invention, e.g., the compounds of this assay can be contacted simultaneously. Alternatively, a subject polypeptide or analog or fragment thereof can first be contacted with a test compound for an appropriate amount of time, following which the PGES-binding partner is added to the reaction mixture. The efficacy of the compound can be assessed by generating dose response curves from data obtained using various concentrations of the test compound. Moreover, a control assay can also be performed to provide a baseline for comparison. In the control assay, isolated and purified subject polypeptide or binding partner is added to a composition containing the PGES-binding partner or subject polypeptide, and the formation of a complex is quantitated in the absence of the test compound.

Complex formation between a subject polypeptide and a PGES-binding partner may be detected by a variety of techniques. Modulation of the formation of complexes can be quantitated using, for example, detectably labeled proteins such as radiolabeled, fluorescently labeled, or enzymatically labeled subject polypeptides or PGES-binding partners, by immunoassay, or by chromatographic detection.

Typically, it will be desirable to immobilize either the subject polypeptide or its binding partner to facilitate separation of complexes from uncomplexed forms of one or both of the proteins, as well as to accommodate automation of the assay. Binding of a subject polypeptide to a PGES-binding partner, can be accomplished in any vessel suitable for containing the reactants. Examples include microtitre plates, test tubes, and micro-centrifuge tubes. In one embodiment, the subject polypeptide or analog or fragment thereof is a fusion protein including a domain that allows the protein to be bound to a matrix. For example, His₆-PGES polypeptides or glutathione-S-transferase/PGES (GST/PGES) fusion proteins can be adsorbed onto Ni- or glutathione sepharose beads (Sigma Chemical, St. Louis, Mo.) or glutathione derivatized microtitre plates, respectively, which are then combined with the PGES-binding partner, e.g. an ³⁵S-labeled PGES-binding partner, and the test compound, and the mixture incubated under conditions conducive to complex formation, e.g. at physiological conditions for salt and pH, though slightly more stringent conditions may be desired. Following incubation, the beads are washed to remove any unbound label, and the matrix immobilized and radiolabel determined directly (e.g. beads placed in scintilant), or in the supernatant after the complexes are subsequently dissociated. Alternatively, the complexes can be dissociated from the matrix, separated by SDS-PAGE, and the level of subject polypeptide or analog or fragment thereof or PGES-binding partner found in the bead fraction quantitated from the gel using standard electrophoretic techniques such as described in the appended examples.

Other techniques for immobilizing proteins on matrices are also available for use in the subject assay. For instance, either subject or its cognate binding partner can be immobilized utilizing conjugation of biotin and streptavidin. For instance, biotinylated subject molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with subject polypeptidecan be derivatized to the wells of the plate, and subject polypeptides trapped in the wells by antibody conjugation. As above, preparations of a PGES-binding protein and a test compound are incubated in the subject polypepitde presenting wells of the plate, and the amount of complex trapped in the well can be quantitated. Exemplary methods for detecting such complexes, in addition to those described above for the GST-immobilized complexes, include immunodetection of complexes using antibodies reactive with the PGES-binding partner, or which are reactive with a subject polypeptide and compete with the binding partner; as well as enzyme-linked assays which rely on detecting an enzymatic activity associated with the binding partner, either intrinsic or extrinsic activity. In the instance of the latter, the enzyme can be chemically conjugated or provided as a fusion protein with the PGES-binding partner. To illustrate, the PGES-binding partner can be chemically cross-linked or genetically fused with horseradish peroxidase, and the amount of polypeptide trapped in the complex can be assessed with a chromogenic substrate of the enzyme, e.g. 3,3′-diamino-benzadine terahydrochloride or 4-chloro-1-napthol. Likewise, a fusion protein comprising the polypeptide and glutathione-S-transferase can be provided, and complex formation quantitated by detecting the GST activity using l-chloro-2,4-dinitrobenzene (Habig et al (1974) J Biol Chem 249:7130).

For processes which rely on immunodetection for quantitating one of the proteins trapped in the complex, antibodies against the protein, such as anti-PGES antibodies, can be used. Alternatively, the protein to be detected in the complex can be “epitope tagged” in the form of a fusion protein which includes, in addition to the subject sequence, a second polypeptide for which antibodies are readily available (e.g. from commercial sources). For instance, the GST fusion proteins described above can also be used for quantification of binding using antibodies against the GST moiety. Other useful epitope tags include myc-epitopes (e.g., see Ellison et al. (1991) J Biol Chem 266:21150-21157) which includes a 10-residue sequence from c-myc, as well as the pFLAG system (International Biotechnologies, Inc.) or the pEZZ-protein A system (Pharmacia, N.J.).

Cell-free assays can also be used to identify compounds which interact with a subject polypeptide and modulate an activity of the subject polypeptide. Accordingly, in one embodiment, a subject polypeptide is contacted with a test compound and the catalytic activity of the subject polypeptide is monitored. In one embodiment, the abililty of the subject polypeptide to bind to and/or to catalyze the conversion of a PGH into a PGE, is determined. The binding affinity of the subject polypeptide to a substrate can be determined according to methods known in the art. Determination of the enzymatic activity of the subject polypeptide can be performed as further described herein.

In a preferred embodiment, the invention provides a screening method which comprises combining a subject polyptide and a substrate together with a test compound in a reaction mixture in conditions sufficient for the subject polypeptide to cleave the target peptide in the absence of the test compound. The method further comprises monitoring the presence of the substrate, the conversion product, such that a difference in the amount of at least one of the substrate or conversion product in the reaction mixture incubated with the test compound relative to a reaction mixture that does not contain the test compound indicates that the test compound is a PGES therapeutic. In an even more preferred embodiment of the invention, the presence and/or the amount of the substrate or the conversion product is determined by spectrometric analysis of the reaction mixture or of a part thereof.

In another embodiment, the invention provides an assay, comprising incubating a purified PGES of the invention with a test compound in the presence of a cyclic endoperoxide substrate of the enzyme under conditions in which PGES catalyzes the conversion of the cyclic endoperoxide substrate into a product which is the 9-keto, 11-alpha-hydroxy form of the substrate; and determining production of said product. A difference in the amount of conversion by PGES in the presence of the test compound relative to a reaction made in the absence of the assay indicates that the test compound is a modulator, e.g., an inhibitor or stimulator, of PGES activity. Product determination may use HPLC, UV Spectrometry, radioactivity detection, or RIA (commercially available for PGE). Product formation may be analyzed by gas chromatography or mass spectrometry or TLC with radioactivity scanning.

Assays can also be developed for identifying compounds which interact and optionally inhibit the generation of, or the conversion of other members of the family of proteins involved in eicosanoid and glutathione metabolism (e.g., the MAPG family). This can be done by a two step screening assay, wherein each step can be conducted as described herein.

Pure PGES polypeptides or analogs or fragments thereof (including fusion polypeptides), e.g., a human naturally-occurring PGES, can be used for crystallization and determination of the crystal structure of the wild-type protein. The coordinates defining the crystal structure can then be loaded onto a computer readable medium and onto a computer, to thereby display the three-dimensional structure of the polypeptide. Accordingly, in a preferred embodiment, the invention provides methods for visualizing the three-dimensional structural of a PGES polypeptide, comprising obtaining crystals of a PGES polypeptide, subjecting the crystals to X-ray diffraction to obtain a set of coordinates defining the three-dimensional structure of the polypeptide; and loading the coordinates onto a computer that has the capability of displaying the three-dimensional structure based on the coordinates. The display of the three dimensional structure on a computer can also be used in the rational design of drugs that may inhibit or stimulate PGES polypeptides. These drugs can then be used for therapeutic or prophylactic treatments, further described below.

Pure PGES polypeptides may also be cocrystallized with a substrate. The three dimensional image of such a complex is useful for designing drugs which modulate the interaction between PGES and a substrate.

The coordinates may also help obtain the three-dimensional structure of a polypeptide related to the PGES polypeptide.

Those skilled in the art will appreciate that the purified PGES-type polypeptides or fusion polypeptides of the invention can be used for diagnostic purposes. For example, they can be used to detect and quantify PGES polypeptides in samples from subjects.

The polypeptides of the invention can also be used as standards in assays for determining the amount of PGES in a sample. Thus, the polypeptides could be part of kits for determining the amount of PGES in a sample, e.g., in a subject.

In another embodiment, the polypeptides of the invention are used to produce antibodies, either monoclonal or polyclonal antibodies. Such antibodies can be used, e.g., for determining the amount of PGES polypeptide in a sample.

The invention provides methods for treating methods for diseases or conditions in patients in need of modulation of PGES and/or PGE activities. In one embodiment, a patient is treated by administering to the patient a modulator of PGES or PGE activity. In one embodiment, a subject having a disease or condition that could benefit from an increase in PGE activity is treated by administration of a pharmaceutically efficient amount of PGE, e.g. PGE₂, prepared in vitro using purified PGE synthase, as described herein, or by administration of the purified PGE synthase to the subject. In another embodiment, a subject having a disease or condition that could benefit from a decrease in PGE activity is treated by administration to the subject of a pharmaceutically efficient amount of an inhibitor of a PGE synthase, identified according to methods provided by the present invention. The compositions can be administered to an animal host, including a human patient, in any suitable manner, e.g., by itself, or in pharmaceutical compositions where it is mixed with suitable carriers or excipient(s) at doses therapeutically effective to treat or ameliorate a variety of disorders, including those characterized by excessive or insufficient PGE activity or PGES activity. A therapeutically effective dose further refers to that amount of the compound sufficient to result in amelioration of symptoms associated with such disorders. Techniques for formulation and administration of the compounds of the instant application may be found in “Remington's Pharmaceutical Sciences,” Mack Publishing Co., Easton, Pa., latest edition.

Prostaglandins have been reported to modulate immune reactions, modulate blood pressure, modulate platelet aggregation, modulate cyclic AMP levels, to inhibit gastric secretion, dilate bronchi, inhibit lipolysis, antagonize vasopressin-induced anti-diarrhesis, constrict the pupil, induce cortical and trabecular bone formation, increase and decrease the intraocular pressure and produce contraction of the uterus. (Ganong, William F., Review of Medical Physiology, 7th ed. (1975), p. 226 (hereinafter “Ganong”)). The naturally occurring prostaglandins all appear to be capable of affecting the control of vascular and other smooth muscle contractions. In the central nervous system, prostaglandins are known to modify responses to certain synaptic transmitters. They have been reported to mimic the actions of some hormones and to inhibit the actions of certain others. (Ganong, p. 226.). Thus, PGE₂ has various activities such as pain inducing activity, inflammatory activity, uterine contractile activity, a promoting effect on digestive peristalsis, an awaking activity, a suppressive effect on gastric acid secretion, hypotensive activity, blood platelet inhibition activity, bone-resorbing activity, angiogenic activity, or the like.

The compounds and compositions of the invention are useful for treating inflammation and pain, e.g., in joint and muscle (e.g., rheumatoid arthritis, rheumatoid spondylitis, osteoarthritis, gouty arthritis, juvenile arthritis, etc.), inflammatory skin condition (e.g., sunburn, burns, eczema, dermatitis, etc.), inflammatory eye condition (e.g., conjunctivitis, etc.), lung disorder in which inflammation is involved (e.g., asthma, bronchitis, pigeon fancier's disease, farmer's lung, etc.), condition of the gastrointestinal tract associated with inflammation (e.g., aphthous ulcer, Chrohn's disease, atopic gastritis, gastritis varialoforme, ulcerative colitis, coeliac disease, regional ileitis, irritable bowel syndrome, etc:), gingivitis, and tumescence after operation or injury, pyrexia, and other conditions associated with inflammation, allergic disease, systemic lupus erythematosus, scleroderma, polymyositis, tendinitis, bursitis, periarteritis nodose, rheumatic fever, Sjogren's syndrome, Behcet disease, thyroiditis, type I diabetes, diabetic complication (diabetic microangiopathy, diabetic retinopathy, diabetic neohropathy, etc.), nephrotic syndrome, aplastic anemia, myasthenia gravis, uveitis contact dermatitis, psoriasis, Kawasaki disease, sarcoidosis, Hodgkin's disease, Alzheimer's disease, kidney dysfunction (nephritis, nephritic syndrome, etc), liver dysfunction (hepatitis, cirrhosis, etc.), gastrointestinal dysfunction (diarrhea, inflammatory bowel diseases, etc.) shock, bone disease characterized by abnormal bone metabolism such as osteoporosis (especially, postmenopausal osteoporosis), hyper-calcemia, hyperparathyroidism, Paget's bone diseases, osteolysis, hypercalcemia of malignancy with or without bone metastases, rheumatoid arthritis, periodontitis, osteoarthritis, ostealgia, osteopenia, cancer cachexia, calculosis, lithiasis (especially, urolithiasis), and cell growth abnormalities, e.g., cancer, such as solid caricinoma, or the like in mammals, preferably in a human.

As those skilled in the art will appreciate, certain biological activities are mediated preferentially by certain of the PGE₂ receptors. PGE₂-sensitive receptors have been sub-divided into four subtypes, EP1, EP2, EP3 and EP4, and these receptors have a wide distribution in various tissues. The effects associated with EP1 and EP3 receptors may be considered as excitatory, and are believed to be mediated by stimulation of phosphatidylinositol turnover or inhibition of adenyl cyclase activity, with resulting decrease in intracellular levels of cyclic AMP. In contrast, the effects associated with EP2 and EP4 receptors may be considered as inhibitory, and are believed to be associated with a stimulation of adenyl cyclase and an increase in levels of intracellular cyclic AMP. Especially, EP4 receptor may be considered to be associated with smooth muscle relaxation, anti-inflammatory or pro-inflammatory activities, lymphocyte differentiation, antiallergic activities, mesangial cell relaxation or proliferation, gastric or enteric mucus secretion, or the like.

A number of disorders in addition to inflammation and pain have been characterized by insufficient or excessive PGE, e.g., PGE₂ activity. In addition, several physiological states which may, from time to time be considered undesired, are also associated with PGE₂ activity. By way of example, but not by way of limitation, such disorders and physiological states which may be treated with the compounds and polypeptides of the invention include but are not limited to neurologic disorders such as Alzheimer's disease, stroke, and acute head injury; colorectal carcinoma; ovulation; preterm labor; endometriosis; implantation; and pulmonary fibrosis.

For example, in obstetrics, prostaglandins have been used for cervical ripening, labor induction and control of post-partum hemorrhage. (Catanzarite, Valerian A. and Gary Aisenbrey, Contemporary OB/GYN (October 1987), p. 29). For cervical ripening, PGE₂ had been given intravenously, orally and vaginally, but the preferred route is intracervically. A PGE₂ gel is now commercially available in Scandinavia, and another PGE₂ gel is being investigated in the United States for Ob/Gyn applications. The PGE₂ gel can also be used for labor induction (3-5 mg of PGE₂, prepared by blending a 20 mg suppository with 60 mL of lubricating jelly and using 9-15 mL of the mixture, is placed in the vagina). (Catanzarite, p. 32). Prostaglandins have also been utilized to control post-partum hemorrhage.

Prostaglandins have been used in gynecology for pregnancy termination. Preparing the cervix with prostaglandin suppository has been found to reduce the incidence of cervical laceration and significant bleeding. (Catanzarite, p. 22). Synthetic analogues of prostaglandin PGE₂, such as 16-16-dimethyl PGE₂ and 9-methylene PGE₂, have proven useful for the induction of first trimester abortions. Such procedures typically use vaginal suppositories containing 20 milligrams PGE₂ into the amniotic sac. Prostaglandin E₂, also known as the “Prostin E2” brand of “dynoprostone,” or Prepidil^{RTM} is available from Upjohn Company in the form of a vaginal suppository. Indications and usage reported by Upjohn are (i) termination of pregnancy from the 12th through the 20th gestational week, (ii) evacuation of the uterine contents in the management of missed abortion or intrauterine fetal death up to 28 weeks of gestational age, and (iii) in the management of non-metastic gestational trophoblastic disease (benign hydatidiform mole). (The Upjohn Co., Prostin E2 product description 810 994 009, October, 1990). Forest Labs provides Cervidil^{RTM} for PEE₂ for similar purposes.

Experimental evidence indicates that prostacyclin and PGE may protect the lungs from injury by endotoxin, such as occurs in the clinical syndrome called adult respiratory distress syndrome (ARDS) Brigham et al., Prostaglandin E₂ attenuation of sheep lung responses to endotoxin, J. Appl. Physiol 64:2568 (1988). Prostacyclin dilates blood vessels in the lung and has been used in therapeutic trials in the treatment of primary pulmonary hypertension, a disease for which there is no acceptable treatment at the present time. Robin, L., Clin. Pulmonary Circ. In Health and Disease; Will, Davison, Weir and Buebrier, eds. Acad. Press, N.Y. pp 491-498 (1987). Prostanoids also dilate airway smooth muscle (bronchodilators) and therefore could be therapeutic in asthma.

PGE can also be administered for the treatment of impotence. U.S. Pat. No. 5,981,593 describes the administration of prostaglandin PGE₂ for treatment of impotance.

Furthermore, PGE can also be administered for treating hypertension. For example, PGE₂ has been shown to dilate blood vessels, i.e., to act as a vasodilator. Since PGH₂ is a vasoconstrictor, administration of PGE synthase to a subject suffering from hypertension would simultaneously increase the amount of vasodilator and decrease the amount of vasoconstrictor.

Set forth below are diseases and conditions that may benefit from the administration of an inhibitor of a wild-type PGES, such as an analog of PGES which is an antagonist (e.g., dominant negative mutant), or a small molecule or other compound identified by a screening assay or by rational drug design, as described herein. For example, inhibition of prostaglandin production would be beneficial in reducing pain in a subject or in reducing immune reactions.

Another disease that may benefit from an inhibitor of prostaglandin production is Alzheimer's Disease (AD), which is characterized by neuritic amyloid plaques, neurofibrillary tangles, neuronal cell loss, loss of synapses, and marked gliosis. Recent findings suggest that the “inflammatory processes” associated with gliosis represent a potential target for therapeutic intervention in the disease. In particular, Joe Rogers and colleagues have presented both retrospective and prospective evidence that non-steroidal anti-inflammatory agents can significantly slow the progress of AD (McGeer and Rogers, 1992, Neurology, 42:447-449; Rogers et al., 1993, Neurology, 43:1609-1611). Indeed, these results have prompted the initiation of anti-inflammatory therapy trials for AD.

Evidence for an “inflammatory component” to gliosis in AD includes increased expression of proinflammatory cytokines such as IL-1.beta. and TNF.alpha. (Griffin et al., 1989, Proc. Nat'l. Acad. Sci. USA, 88:7611-7615; Dickson et al., 1993, Glia, 7:75-83; Lapchak and Araujo, 1993, Soc. Neurosci. Abstr., 19:191) and the presence of activated complement components (McGeer et al., 1989, Neurosci. Let., 107: 341-346; Johnson et al., 1992, Neurobiol. Aging, 13:641-648; Walker and McGeer, 1992 Mol. Brain Res., 14:109-116). It should be noted that gliosis and the presence of proinflammatory cytokines are not limited to AD. Rather, they are a feature of many insults to and disease of the central nervous system including (but not limited to) acute head injury, stroke, spinal cord injury, multiple sclerosis, HIV infection of the brain and other viral encephalopathies, and most neurodegenerative disorders (e.g. Huntington's disease, Parkinson's disease, and amyotrophic lateral sclerosis).

Colorectal carcinoma is a leading cause of death in westernized countries. Prostaglandins have been correlated with carcinogenesis in general and more specifically with colorectal cancer, Marnett, 1992, Cancer Research, 52:5575-5589. In several clinical trials, aspirin use was associated with decreased colon tumor growth and death, Thun et al., 1991, N. Engl. J. Med., 325:1593-6; Kune, et al., 1988, Cancer Res., 48:439-404. Accordingly, administration of inhibitors of PGES may help treating carcinomas, e.g., colorectal carcinoma.

Ovulation has in a broad sense can be viewed as an inflammatory process initiated by the LH surge during the menstrual cycle, Espey, 1980, Biol. Reprod, 22:73-106. NSAIDs have been shown to inhibit ovulation in a number of model systems, Espey, 1982, Prostaglandin, 23:329-335. By inhibiting prostaglandin formulation and interrupting the inflammatory response ovulation is halted. Accordingly, inhibitors of PGES polypeptides may be used for blocking ovulation, and for use, e.g., as a contraceptive.

As described above, prostaglandins induce labor, which, when non-desired, e.g., in preterm labor, which is a significant clinical problem. Kelly, 1994, Endocrine Reviews, 15(5):684-706. Prostaglandins are intimately involved in myometrium contraction of normal labor, Williams Obstetrics, Cunningham, MacDonald, Gant, Leveno, and Gilstrap (eds) Williams Obstetrics 19th Ed. Appleton and Lange, Norwalk Conn., 1993. Accordingly, inhibitors of PGES may be used to prevent preterm labor, and complications arising therefrom.

Dysmenorrhea and endometriosis are common, painful problematic conditions for women. It is well known that NSAIDs are extremely effective at treating dysmenorrhea and endometriosis pain by inhibiting prostaglandin production. Accordingly, inhibitors of PGES could prevent the production of prostaglandins and alleviate these conditions.

As described above, PGE₂ is a vasodilator. Accordingly, local application of inhibitors of PGE could prevent dilation of blood vessels and consequences resulting therefrom.

The invention also provides methods of treating diabetes, other autoimmune conditions, and conditions involving dysfunctional apoptotic processes. Further embodiments of the subject invention provide to therapeutic methods based on modulation of cell death mechanisms and events. In a specific embodiment, individuals at risk for cancer or to develop autoimmune diseases will display a prostaglandin related resistance to cell death upon stimulation of cells by chemical factors including, but not limited to, TNF-alpha and FAS ligand. By intervening in this process by, for example, the administration of inhibitors of PGES activity, it is possible to facilitate the completion of cell death events to eliminate inappropriate cells from the biological milieu. In this way, autoimmune T-cells can be removed through the apoptic mechanism upon stimulation by appropriate chemical signals or immunization with disease-related target antigens such as insulin or GAD. Similarly, cancer cells can also proceed to appropriate cell death, thereby reducing or prevening tumors and/or other inappropriate cellular proliferation. Thus, a further specific embodiment of the subject invention concerns the administration of inhibitors of PGES, or its biological activities, to effect a modulation of programmed cell death such that self-destructive T-cells and/or cancer cells are removed to treat autoimmune or cancer conditions.

The compounds of the invention may be targeted to specific sites, e.g., sites of inflammation, by direct injection to those sites, e.g., joints, in the case of arthritis. Compounds designed for use in the central nervous system should be able to cross the blood brain barrier or be suitable for administration by localized injection. Similarly, compounds specific for the bladder can be directly injected therein. Compounds may also be designed for confinement in the gastrointestinal tract for use against disorders such as colorectal carcinoma. In addition, the compounds of the invention which remain within the vascular system may be useful in the treatment of vascular inflammation which might arise as a result of arteriosclerosis, balloon angioplasty, catheterization, myocardial infarction, vascular occlusion, and vascular surgery. Such compounds which remain within the bloodstream may be prepared by methods well known in the art including those described more fully in McIntire, 1994, Annals Biomed. Engineering, 22:2-13.

Pharmaceutical compositions suitable for use in the present invention include compositions wherein the active ingredients are contained in an effective amount to achieve its intended purpose. More specifically, a therapeutically effective amount means an amount effective to treat the development of or to alleviate the existing symptoms of the subject being treated. Determination of the effective amounts is well within the capability of those skilled in the art, especially in light of the detailed disclosure provided herein.

For any compound used in the method of the invention, e.g., a small molecule or a polypeptide, the therapeutically effective dose results in suitable treatment of the patient can be estimated initially from cell culture assays. For example, a dose can be formulated in animal models to achieve a circulating concentration range that includes the IC50 (the dose where 50% of the cells show the desired effects) as determined in cell culture. Such information can be used to more accurately determine useful doses in humans.

A therapeutically effective dose refers to that amount of the compound that results in treatment of symptoms or a prolongation of survival in a patient. As those skilled in the art will recognize, the dosage may vary within this range depending upon the dosage form employed and the route of administration utilized. The exact formulation, route of administration and dosage can be chosen by the individual physician in view of the patient's condition. (See e.g. Fingl et al., 1975, in “The Pharmacological Basis of Therapeutics”, Ch. 1 p1). Dosage amount and interval may be adjusted individually to provide plasma levels of the active moiety which are sufficient to maintain the desired effects.

In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

Pharmaceutical compositions of the present invention may be manufactured in a suitable manner, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in any conventional manner using one or more physiologically acceptable carriers comprising excipients and auxiliaries which facilitate processing of the compounds into preparations which can be used pharmaceutically. Proper formulation is dependent upon the route of administration chosen.

For injection, the agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the compounds may be dissolved or suspended in suitable liquids, such as fatty oils, liquid paraffin, or liquid polyethylene glycols. In addition, stabilizers may be added. All formulations for oral administration should be in dosages suitable for such administration.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multidose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the compounds in water-soluble form. Additionally, suspensions of the compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds of the invention is a cosolvent system comprising benzyl alcohol, a nonpolar surfactant, a water-miscible organic polymer, and an aqueous phase. Naturally, the proportions of a co-solvent system may be varied considerably without destroying its solubility characteristics. Furthermore, the identity of the co-solvent components may be varied.

Alternatively, other delivery systems for hydrophobic pharmaceutical compounds may be employed. Liposomes and emulsions are well known examples of delivery vehicles or carriers for hydrophobic drugs. Liposomes can be targeted to specific sites in the body by modifying the outer surface of the lipose, e.g., by linking an antibody that recognizes an antigen on the surface of target cells. Certain organic solvents such as dimethylsulfoxide also may be employed. Additionally, the compounds may be delivered using a sustained-release system, such as semipermeable matrices of solid hydrophobic polymers containing the therapeutic agent. Various of sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Compositions including prostaglandins preferably contain at least one surfactant in order to help solubilize or disperse the prostaglandin in the composition. Surfactants also inhibit or prevent the adsorption of the prostaglandin on to the container walls. The surfactant may be any pharmaceutically acceptable surfactant, such as pharmaceutically acceptable cationic, anionic or nonionic surfactants. Examples of suitable surfactants include polyethoxylated castor oils, such as those classified as PEG-2 to PEG-200 castor oils, as well as those classified as PEG-5 to PEG-200 hydrogenated castor oils. Such polyethoxylated castor oils include those manufactured by Rhone-Poulenc (Cranbury, N.J.) under the Alkamuls™ brand, those manufactured by BASF (Parsippany, N.J.) under the Cremophor™ brand, and those manufactured by Nikko Chemical Co., Ltd. (Tokyo, Japan) under the Nikkol brand. Preferred polyethoxylated castor oils are those classified as PEG-15 to PEG-50 castor oils, and more preferred are PEG-30 to PEG-35 castor oils. It is most preferred to use those polyethoxylated castor oils known as Cremophor™ EL and Alkamuls™ EL-620. Preferred polyethoxylated hydrogenated castor oils are those classified as PEG-25 to PEG-55 hydrogenated castor oils. The most preferred polyethoxylated hydrogenated castor oil is PEG-40 hydrogenated castor oil, such as Nikkol HCO-40.

Many of the compounds of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents that are the corresponding free base forms.

Suitable routes of administration may, for example, include topical, oral, rectal, transmucosal, transdermal, or intestinal administration; parenteral delivery, including intramuscular, subcutaneous, intramedullary injections, as well as intrathecal, direct intraventricular, intravenous, intraperitoneal, intranasal, or intraocular injections.

Alternatively, one may administer the compound in a local rather than systemic manner, for example, via injection of the compound directly into an affected area, often in a depot or sustained release formulation.

Furthermore, one may administer the drug in a targeted drug delivery system, for example, in a liposome coated with an antibody specific for affected cells. The liposomes will be targeted to and taken up selectively by the cells.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. Compositions comprising a compound of the invention formulated in a compatible pharmaceutical carrier may also be prepared, placed in an appropriate container, and labelled for treatment of an indicated condition. Suitable conditions indicated on the label may include treatment of a disease such as one characterized by insufficient, aberrant, or excessive PGES activity or PGE₂.

The invention provides kits for in vitro preparation of PGES polypeptides, which may comprise a nucleic acid encoding a PGES polypeptide, bacteria for expressing the nucleic acid, chromatographic material for purifying PGES polypeptide, and/or control PGES polypeptide. Other kits comprise one or more components for preparing prostaglandin in vitro. Such kits may comprise purified preparation(s) of a PGES polypeptide or analog or fragment thereof and a PGH and optionally buffers.

The invention further comprises kits for diagnostic or therapeutic purposes. Therapeutic kits may comprise a compound identified by methods of the invention, or a therapeutic amount of a polypeptide of the invention and, e.g., a pharmaceutical vector for topical or other type of administration. Other kits may comprise one or more pharmaceutical compositions comprising a PGE synthesized according to the methods of the invenion. Diagnostic kits may comprise purified polypeptide of the invention for measuring the amount of antibody against PGES in a sample.

Other kits contain a purified polypeptide of the invention to serve as a standard (or positive or negative control) in an assay for testing the activity of a PGES polypeptide, such as a polypeptide of cells of a subject. Similarly, the purified polypeptide can serve as a standard for determining the presence and/or amount of PGES polypeptide present in a sample. Accordingly, exemplary kits comprise a solution of purified PGES polypeptide of the invention, e.g., in a buffer comprising glycerol and/or reduced glutathione. The kit may comprise different solutions of different concentrations or activity. The kit may comprise several samples, which may be kept frozen or at 4° C.

The present invention is further illustrated by the following examples which should not be construed as limiting in any way. The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2^{nd} Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization(B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

### EXAMPLES

### Example 1

### Cloning, Expression and Purification of Histidine-Tagged Human Prostaglandin E Synthase (His₆-hPGES) in E. coli

The coding nucleotide sequence of human PGES (hPGES) having SEQ ID NO: 1 was amplified by PCR from the expressed sequence tag clone 143735 and inserted into the expression vector pSP19T7LT (Jakobsson et al. (1999) PNAS 96:7220). A sequence of six histidine residues had been introduced in the vector at the NdeI site where the end coding for the N-terminal of hPGES was inserted. The nucleic acid sequence encoding the fusion polypeptide is set forth in SEQ ID NO: 3 and encodes the polypetides set forth in SEQ ID NO: 4 (both of which are also shown in FIG. 2A). Recombinant His₆-hPGES was expressed in E. coli BL21(DE3) (which harbored the plasmid pLysSL (Studier (1991) J. Mol. Biol. 219:37) as follows. An overnight culture of BL21(DE3) cells in LB broth containing ampicillin (100 μg/ml) and chloramphenicol (20 μg/ml) was diluted 1:100 into 1-2L of Terrific roth medium containing ampicillin and chlormaphenicol (100 and 20 μg/ml, respectively). The culture was grown at 37° C. in Terrific Broth medium until OD₆₀₀ was 0.45-0.60 when expression of hPGES was induced by 3 mM isopropyl β-D-thiogalactopyranoside (IPTG). The culture was thereafter grown for another 3 h at 37° C. followed by harvesting by centrifugation and stored at −20° C. until further processing.

Frozen BL21 (DE3) cell pellets were thawed on ice and suspended in a solution containing 15 mM Tris-HCl, pH 8.0, 0.25 M sucrose, 0.1 mM EDTA, 1 mM GSH (20 ml for a pellet of 1L culture. The cells were lysed upon freeze-thawing by the extrusion of internal T7 lysozyme expressed from the pLysSL plasmid (Studier et al., supra). NA was hydrolyzed by the addition of 10 mM MgCl₂ and 0.4 μg/ml Dnase and incubation on ice for 30 minutes. The lysate was thereafter sonicated by six 30-s sonication pulses from a MSE Soniprep 150 sonicator (MSE Scientific Instruments, Sussex, UK) at 60% power. Unbroken cells and debris were separated by low speed centrifugation at 5,000 g for 10 minutes. Membrane fraction was pelleted by ultracentrifugation at 250,000 g for one hour, and resuspended in 20 mM sodium phosphate buffer, pH 8.0, at a concentration of 10-20 mg/ml membrane protein. The membrane fractions were stored at −70° C. A 11 expression culture yielded 60-100 mg of total membrane protein.

Frozen E. coli BL21(DE3) membrane fraction containing recombinant His₆-hPGES was thawed and membrane proteins were solubilized in 10 or 50 mM sodium phosphate buffer, pH 8.0, 150 mM NaCl, 4% Triton X-100, for 30 min. on ice with stirring at a protein concentration of 5 mg/ml. Insoluble material was separated by ultracentrifugation at 100,000 g for 30 min. The cleared supernatant was filtered through a 0.45 μm filter.

Proteins were visualized by Western blot analysis. 1 or 10 μg of protein samples (as indicated in FIG. 2B) were separated by SDS-PAGE and electrophoretically transferred to a PVDF membrane. Nonspecific sites on the membrane were blocked with 5% nonfat dry milk in TBST. The membrane was probed with polyclonal PGES peptide antiserum (Jakobsson et al., supra) followed by horseradish peroxidase conjugated donkey anti-rabbit secondary antibodies (Amersham Pharmacia Biotech). Bands were detected with ECL-Plus enhanced chemiluminescence staining kit (Amersham Pharmacia Biotech). This procedure is described in Jakobssen et al., supra.

The results, which are shown in FIG. 2B, illustrate that the present method provides for efficient extraction and solubilization of the PGES. Accordingly, hPGES was completely extracted by 4% Triton X-100 with preserved enzymatic activity in the solubilized extract (see below).

### Example 2

### Solubilization and PGES Activity

Membrane fractions were solubilized as described above in different buffer compositions to investigate suitable conditions for solubilization of the hPGES in an active state for the subsequent purification experiments. The following four different media were tested:

1. 50 mM sodium phosphate buffer, pH 8.0, 150 mM NaCl, 4% Triton X-100

2. 50 mM sodium phosphate buffer, pH 8.0, 150 mM NaCl, 4% Triton X-100+10% glycerol

3. 50 mM sodium phosphate buffer, pH 8.0, 150 mM NaCl, 4% Triton X-100+1 mM reduced glutathione

4. 50 mM sodium phosphate buffer, pH 8.0, 150 mM NaCl, 4% Triton X-100+1 mM reduced glutathione+10% glycerol

PGES activity was assayed in 50 mM sodium phosphate buffer, pH 7.4, in the presence of 2.5 mM reduced glutathione (GSH; obtained from Sigma Chemical Co. (St. Louis, Mo.) and either zero; 0.2; 0.4 or 0.6 mg/ml of purified hPGES. The reaction was started by the addition of 10 μM PGH₂, and the samples were incubated for 1 minute at 0° C. until terminated by 20 mM FeCl₂, 40 mM citric acid and 2.1 μM 11-β. PGE₂, lowering the pH to 3. PGH₂ was prepared by brief incubation of arachidonic acid with suspensions of the microsomal fraction of homogenate of the sheep vesicular gland essentially as described in Hamberg et al. (1974) PNAS 71:345 or Hecker et al. (1987) Biochem. Pharmacol. 36:851. L-tryptophan (5 mM) was used as electron donor. Purification was accomplished by straight-phase HPLC using a solvent system of 2.5% 2-propanol-hexane containing 0.002% acetic acid. Solid phase extraction using C₁₈ Chromabond columns was performed immediately. The samples were eluted with acetone, dried and dissolved in acetonitrile (⅓ by vol. in H₂O) and the formation of PGE₂ was analyzed by reverse-phase HPLC on a C₁₈ (3.9×150 mm, 4 μM particle size) column and UV detection at 195 nm.

The results, which are shown in FIG. 3, show that it is advantageous to include GSH and glycerol in the solubilization medium for preserving the activity of the solubilized hPGES.

### Example 3

### Stability of PGES Activity in Solubilized Membranes in the Presence of Glycerol and GSH

This Example describes conditions for preserving the activity of PGES.

Membrane fractions were solubilized in 50 mM sodium phosphate buffer, pH 8.0, 150 mM NaCl, 4% Triton X-100, 10% glycerol and 1 mM GSH and stored at 4° C. Samples were assayed for PGES activity as described above after 0, 3, 6, 24 and 240 h (10 days) at 4° C.

The results, which are shown in FIG. 4, demonstrate that the activity of the solubilized recombinant hPGES is preserved for a prolonged time in the presence of glycerol and GSH. After 24 h at 4° C., 70% of the conversion remains and even after such a long time as 10 days, 40% of the original activity remains. On the contrary, in the absence of glycerol and GSH, the PGES activity in BL21 (DE3) membranes rapidly declines within a period of 24 h.

### Example 4

### Purification of hPGES

This Example demonstrates that recombinant hPGES of the invention can be purified from isolated membrane fractions by a two-step combination of hydroxyapatite-followed by immobilized metal ion chromatography.

Frozen E. coli BL21(DE3) membrane fractions containing recombinant His₆-hPGES were thawed and solubilized as described above by 4% Triton X-100 in 10 mM sodium phosphate buffer, pH 8.0, 150 mM NaCl, 10% glycerol, 1 mM reduced glutathione (GSH) at a protein concentration of 5 mg/ml. The phosphate concentration was lowered to 10 mM to promote binding of proteins to the hydroxyapatite in the following step. In this particular experiment a fraction containing 240 mg total membrane protein was used.

The solubilized membrane protein mixture was mixed with hydroxyapatite, (HA), (Bio-Gel HTP, Bio-Rad) 1 g/100 mg membrane protein, that had been equilibrated with 10 mM sodium phosphate buffer, pH 8.0, 150 mM NaCl, 1 mM GSH, 10% glycerol, 10 mM imidazole, 0.2% reduced Triton X-100 (Sigma X-100 RS). Reduced Triton was used to minimize the UV absorption of the buffer to facilitate detection of eluted proteins. After a 10 minutes incubation on ice, the HA was pelleted by a short centrifugation pulse and the unbound fraction (flow-through) containing a large part of the His₆-hPGES was removed and cleared by centrifugation (1500 g, 3 min) and filtration (0.45 mm).

The cleared, unbound fraction from the HA was immediately loaded on a 1 ml HiTrap Chelating Sepharose column (Amersham-Pharmacia Biotech) (a 1 ml column was used for up to 300 mg membrane proteins as start material) that had been charged with NiCl₂ and equilibrated with 10 mM sodium phosphate buffer, pH 8.0, 150 mM NaCl, 10 mM imidazole, 1 mM GSH, 10% glycerol, 0.2% reduced Triton X-100 (start buffer) at a flow rate of 1 ml/min. After loading, the column was washed with start buffer until all unbound proteins were eluted. Thereafter, 60 mM imidazole was added to wash out unspecifically bound proteins. Finally, the histidine-tagged protein that had bound to the affinity column was eluted by the addition of 300 or 350 mM imidazole. The elution profile is shown in FIG. 5A. The eluted peak was immediately desalted into 20 mM Na-Phosphate-buffer pH 7.5, 50 mM NaCl, 10% glycerol, 1 mM GSH and 0.2% Triton X-100 on a HiPrep 26/10 Desalting column (Amersham-Pharmacia Biotech) at a flow rate of 8 ml/min.

To improve purity, the desalted protein peak was reloaded and purified a second round on the Ni-column. All steps were performed identically as above except for the subsequent desalting that was made with PD10 desalting columns.

The recovery after the second Ni-column was 0.49 mg (0.2% of the total amount of start material) pure His₆-PGES distributed in 7 ml with 0.07 mg/ml. After the first Ni-affinity column, 0.57 mg was recovered in 3 ml with 0.19 mg/ml. Thus, only 14% (0.08 mg) was lost in the second Ni-column and part of that were probably impurities such as other unspecifically bound proteins. The purified protein was further analyzed by Western blot and PGES activity determination (see below) with positive results from both experiments.

The amount and identity of proteins eluted from the column was visualized by subjecting samples of eluate fractions to SDS-PAGE (15% gel) and silver staining of the gel. A photograph of the gel is shown in FIG. 5. A 17.5 kDa protein purified to apparent homogeneity was obtained. The yield was 0.2-1 mg purified protein from isolated membrane fractions and 1.0-3.5 mg when purifying from whole cell lysate (see below) per IL BL21(DE3) expression culture. The protein was identified as PGES by immunoblot analysis using rabit polyclonal peptide antiserum directed against PGES and PGES activity determination (see below). The molecular mass of the purified protein was calculated from its electrophoretic mobility relateive to standards to 17.5 kDa, which is in agreement with the therotical molecular mass of the his₆-hPGES (17.9 kDa). Identical results were obtained when calculating from a stsained SDS-PAGE or a Western blot.

A major part of the hPGES did not bind to the HA and could thus be recovered in the flow-through fraction and thereafter be well purified on the Ni-column. The HA step is necessary, however, since it appears that some proteins, that otherwise interfere in the Ni-affinity chromatography, are specifically bound and thus removed by the HA. One-step purification by Ni-affinity chromatography was unsuccessful since many unspecifically bound proteins co-eluted with the His₆-hPGES and a selective exclusion of these could not be achieved.

Another metal-chelating resin (Ni-NTA Superflow from QIAGEN) has been tested for the purification of His₆-hPGES. The Ni-NTA resin was found to have a lower affinity for the his-tagged hPGES than the Chelating Sepharose, however, and all purification experiments are now made using Chelating Sepharose columns. The main reason for the test of another affinity resin was primarily the documented stronger resistance to reducing agents of the Ni-NTA agarose, important when GSH has to be included in all buffers to preserve the functionality of the PGES. It was found, however, that buffers containing 1 mM GSH can be used with Chelating Sepharose columns without any apparent damage to the resin. However, even if not as efficient, the resin from Qiagen (Chatsworth, Calif.) can also be used according to the method of the invention to purify PGES.

### Example 5

### hPGES Content in Membrane Fractions

Since purified PGES protein was now available, quantitative Western blots utilizing the purified His₆-hPGES as standards to determine the amount of recombinant protein expressed in the bacterial membrane could be made. Accordingly, quantitative Western blots were performed to determine the amount of recombinant His₆-hPGES expressed in BL21DE3 cells. Western blot was performed as described above (FIG. 2). The band intensity was determined by densitometry.

A photograph of the Western blot is shown in FIG. 6. The response was linear up to 20 ng. The content of PGES was varying between 0.5-2% of the total membrane protein in different membrane batches. This also illustrates that the purification methods provided by the invention are efficient.

### Example 6

### Activity and Stability of the Purified His₆-hPGES

The activity of the purified His₆-hPGES was assayed on a sample of protein. The remaining protein was divided in two parts, one was stored at 4° C. for 5 days, and one was frozen and stored at −20° C. for the same time. The PGES activity in the three samples was compared with that in a membrane fraction without solubilization. PGES activity was determined, as described above, for various amounts of protein. The samples were incubated for 1 min on ice or at 37° C. before stopping the reaction. The activity of the purified proteins was compared with the activity in BL21(DE3) membrane fraction without solubilization.

The results are shown in FIG. 7. The PGES activity of the recombinant histidine-tagged enzyme was stable and does not appear to be affected by solubilization or freeze-thawing. Thus, PGES protein can be prepared, purified and stored, without significant loss of activity. Larger amounts of PGES may also be produced and stored for a longer period. Storage for up to at least two months at −20° C. does not seem to result in any loss of activity in that batch. After five days of storage at 4° C., the activity was unchanged compared to the freshly prepared enzyme and after 34 days at 4° C., about 50% of the original activity remained. Samples intended for long time storage were kept under nitrogen to prevent damage of the protein by oxidation. PGES in unsolubilized membrane fraction (where the his₆-hPGES content had been determined by quantitative immunoblotting) showed a similar activity as the solubilized, purified enzyme.

The specific activity of the purified His₆-hPGES was calculated to 15 μmol min⁻¹ mg⁻¹ at 0° C. and 167 μmol min⁻¹ mg⁻¹ at 37° C. for the glutathione dependent conversion of PGH₂ to PGE₂. This may be compared with the published Vmax of the recently identified cytosolic PGES (0.19 μmol min⁻¹ mg⁻¹ at 24° C.) (Tanioka et al. (2000) J. Biol. Chem. 275:32775), which is about 1,000 times lower.

Thus, the specific activity of purified his₆-hPGES for the conversion of PGH₂ to PGE₂ is the highest activity so far reported for any PG-synthase (Table 3). The K_{M} for PGH₂ (0.16 mM) is comparable to other PG-synthases reported, whereas the turnover number and catalytic efficiency k_{cat}/K_{M}, are magnitudes higher (Table 3).

### Example 7

### Purification From BL21DE3 Whole Cell Lysate Without Membrane Separation

Since isolation of polypeptides from membrane fractions is usually more tedious than their isolation from whole cell lysates, it was investigated whether it would be possible to extract and purify the recombinant His₆-hPGES directly from a BL21(DE3) whole cell lysate. This should allow the overall time for the purification process to be considerably shortened and the losses minimized.

The following method was used to isolated PGES from a whole cell lysate. Recombinant His₆-hPGES was expressed in E. coli BL21(DE3) cells as described above. A frozen cell pellet from a 1 liter His₆-hPGES expression culture was thawed and resuspended in 20 ml of 10 mM sodium phosphate buffer, pH 8.0, 150 mM NaCl, 10% glycerol, 1 mM GSH. These cells were self-lysed upon freeze-thawing by the extrusion of the internal T7 lysozome that had been expressed by the pLysSL vector. The viscous whole cell lysate was sonicated in an ice water bath by the instrument described above in 15 seconds pulses until homogeneous, solubilized by the addition of an equal volume of 10 mM sodium phosphate buffer, pH 8.0, 150 mM NaCl, 10% glycerol, 1 mM GSH plus 8% Triton X-100 and gentle stirring on ice for 30 minutes. The solubilized lysate was cleared from insoluble material by ultracentrifugation and filtration as described for the membrane fraction. His₆-hPGES was thereafter purified from the solubilized lysate by hydroxyapatite and Ni-affinity chromatography as described for the protein solubilized from the membrane fraction.

Samples of the eluted proteins were subjected to SDS-PAGE (15% gel) and the gel was silver stained. A photograph of the gel is set forth as FIG. 8. The results show that, starting with solubilization of whole cell lysate, without first separating the membranes, worked equally well as with membrane fractions for the purification of His₆-hPGES. Actually, the yield was even higher, 3.4 mg pure His₆-hPGES eluted in two peaks, 300 mM (large, 0.35 mg/ml, lane 6, FIG. 8)) and 500 mM (small, 0.04 mg/ml, Lane 7, FIG. 8) imidazole peak. In later experiments, the concentration of imidazole used for elution of the His₆-hPGES was increased to 350 mM resulting in elution of all the protein in a single peak. PGES activity was determined and was in both peaks equal to that determined with His₆-hPGES purified from membrane fractions.

Thus, a large amount of time is saved by this procedure, making it possible to express and purify considerable amounts of protein from small-scale cultures relatively fast, e.g., in four hours.

### Example 8

### Purification Upscale

In an attempt to scale up the purification process to increase the amount and concentration of the purified His₆-hPGES, an experiment starting with frozen cell pellets from 51 of expression culture was undertaken. All solubilization and chromatography steps were performed substantially as described above for the purification from whole cell lysate with all volumes and column sizes 5 times larger.

A five times scale-up of the purification process did not yield five times more pure protein. The elution peak from the first Ni-column (5 ml Chelating Sepharose) was still containing too many impurities so the peak was reloaded on a 1 ml column to increase protein purity and concentration. Only a part of the His₆-hPGES from the first peak actually bound to the second Ni-column, however, and finally four reloads were made on the Ni-affinity column. All those resulted in elution of a portion of pure His₆-hPGES but the concentration could thus not be increased as much as desired. The total yield of purified His₆-hPGES was 4.9 mg distributed in 5.5 ml of 0.57 mg/ml (2nd Ni-column), 5.5 ml of 0.21 mg/ml (3^{rd} Ni-column) and 4 ml of 0.14 mg/ml (4^{th} Ni-column).

Samples from various peaks were subjected to SDS-PAGE (15% gel) and the gel was then silver stained (FIG. 9) or subject to Western blot analysis (FIG. 10). As can be seen in FIG. 9 (lane 6) and in the Western blot (FIG. 10, lane 7), there is still a large amount of His₆-hPGES unbound in the flow-through fraction even after four runs on Ni-columns. The reason for this is not clear but a probable explanation may be that the small 1 ml-column does not have the capacity to bind more protein and thus becomes overloaded. As can be seen from the Western blot (FIG. 10, lane 9), all the His₆-hPGES is retained by the larger 5 ml Ni-column in the first affinity chromatography step.

### Example 9

### Determination of K_{M} and Vmax for PGH₂

Activities were assayed with purified His₆-hPGES, 1 mg/ml at 0° C. and 0.5 mg/ml at 37° C. in 50 mM sodium phosphate buffer, pH 7.4, as described above with 2.5 mM GSH and varying PGH₂ concentration. All points were made in triplicate. The Michaelis-Menten constants K_{M} and Vmax (maximum velocity) were derived by hyperbolic regression with the computer program GraphPad Prism software 3.0 or 3.02 (GraphPad Software, Inc., San Diego, Calif.).

The graphs are shown in FIG. 11. The K_{M} for PGH₂ was 50±8.9 μM at 0° C. and 163±42 μM at 37° C. Vmax was 0.25±0.013 μM s⁻¹ at 0° C. and 1.39±0.16 μM s⁻¹ at 37° C.

When GSH was substituted by other sulfhydryl compounds such as N-acetylcysteine, dithiothreitol or 2-mercaptoethanol, or when the purified his₆-hPGES was heated at 100° C. for 10 minutes, no PGES-activity could be detected. Pre-incubation of purifed his₆-hPGES with 5 mM N-ethylmaleimide in the absence of GSH for 1 minute reduced the PGES activity by almost 100%.

### Example 10

### Substrate Specificity of the hPGES

The activity of purified His₆-hPGES has been assayed with PGH₂, LTA4, 1-chloro-2,4-dinitrobenzene (CDNB), PGG2 and 5-hydroperoxyeicosatetraenoic acid (5-HPETE).

LTC₄ synthase activity was assayed with up to 100 μg/ml purified His₆-hPGES that was incubated with 30 μM LTA4 and 10 mM GSH at pH 7.4 as described in (Jakobsson et al. (1996) J. Biol. Chem. 271: 22203). No activity above background could be detected with up to 100 μg/ml of the purified enzyme, 100 times more protein than is normally used for assaying PGES activity (see Table I).

Glutathione transferase (GST) activity (glutathione conjugation) was assayed with the classical model substrate for GSTs, p-chloro-2,4-dinitrobenzene (CDNB). Purified His₆-hPGES (0.1 mg/ml) was incubated with 5 mM GSH (glutathione, Sigma Chemical Co., St. Louis, Mo.) and 1 mM CDNB (Sigma Chemical Co., St. Louis, Mo.) in 0.1 M potassium phosphate buffer, pH 6.5, 0.1% Triton X-100 at 30° C. Formation of product was detected at 340 nm (molar absorption coefficient for the product, GS-DNB, is 9.6 mM⁻¹ cm⁻¹).

The results indicate that purified His₆-hPGES does possess a small but significant GSH-CDNB conjugating activity (see Table I). The Km for CDNB could not be determined, since the GST activity was linear up to 2 mM CDNB. When the purified His₆-hPGES was pre-incubated with 5 mM N-ethylmaleimid in the absence of GSH for 1 minute, the enzyme activity was reduced by 50%. When the purified His₆-hPGES was incubated with LTA4 or heated at 100° C. for 10 minutes, no GST activity could be detected.

Other activities related to the MAPEG superfamily were tested. Both MGST2 and MGST3 catalyze the glutathione-dependent reduction of 5-HPETE into 5-hydroxyeicosatetraenoic acid (5-HETE) (Jakobssen et al. (1997) J. Biol. Chem. 272: 22934). Thus, to measure the peroxidase activity of PGES, 5-HPETE (10 μM) (Biomol, Plymouth Meeting, Pa.) was assayed in 100 μl of reaction mixture, containing 0.1 M potassium phosphate buffer, pH 7.4; 5 mM glutathione; 0.005% (w/v) bovine serum albumin and 0.025-0.1 mg/ml of purified His₆-hPGES. The reaction mixture was incubated for 3 minutes at 37° C. The reaction was terminated by addition of 200 μl acetonitrile containing 0.2% acetic acid. 100 μl water was then added and the protein was removed by centrifugation at 14,000 g for 1 minute. An aliquot (200 μl) was then analyzed by RP-HPLC equipped with a Nova-Pak C18 (3.9×150 mm, 4 μm particle size), obtained from Waters. The mobile phase was water, acetonitrile and trifluoroacetic acid (40:60:0.007, by vol.). The flow rate was 1.0 ml/min and the products were quantified based on the peak areas at 236 nm from known amounts of injected 5-HPETE and 5-HETE. The results indicate that PGES has a low but significant glutathione peroxidase activity (Table I). The glutathione-dependent peroxidase activity disappeared when the purified His₆-hPGES was heated at 100° C. for 10 minutes.

His₆-PGES also catalyzed the reduction of 15-hydroperoxy-PGE₂ in the presence of GSH although at a rate magnitude lower than the production of 15-hydroperoxy-PGE2. Furthermore, purified his₆-hPGES catalyzed GSH-dependent peroxidase activity towards cumene hydroperoxide (0.17 μmol/min mg). As a control, the enzyme was boiled for 10 minutes, which abolished all activity. Peroxidase activity with cumene hydroperoxide as substrate was determined by a coupled assay with 0.05 mg/ml purified his₆-hPGES, 1 mM GSH, 0.2 mM NADPH, 0.5 mM cumene hdyroperoxide (dissolved in alcohol) and an excess amount of glutathione reductase as the linear decrease in NADPH absorption at 340 nm (Wendel (1981) Methods Enzymol. 77:325).

It was then invastigated whether purified his₆-hPGES catalyzed the siomerization of PGG₂ into 15-hydroperoxy-PGE₂. PGG₂ was assayed as a substrate of PGES in 100 μl reaction mixture, containing 0.1 M potassium phosphate buffer pH 7.4; 2.5 mM reduced glutathione; 10-8001M PGG₂ and 0.2-2.0 μg/ml of purified His₆-hPGES. PGG₂ was prepared similarly to PGE₂ (described above), omitting the L-tryptophan. The reaction was terminated by lowering the pH to 3 by adding 6 μl HCl (1M). Then, 45 μl acetonitrile was added and centrifugation was performed at 14,000 g, for 1 minute to remove the protein. The stop solution did not contain FeCl₂, since it degrades all the peroxides, including 15-Hydroperoxy PGE₂. In order to determine the formation of PGE₂ and 15-Hydroperoxy PGE₂, an aliquot (100 μl) was analyzed by RP-HPLC, combined with UV detection at 195 nm. The products were quantified based on the peak areas from known amounts of injected prostaglandins.

The results indicate that His₆-hPGES catalyzes the specific conversion of PGG₂ into 15-Hydroperoxy PGE₂ (15-HP PGE₂) in the presence of reduced glutathione. This is presented in a typical chromatogram (FIG. 12B) as compared to the buffer control (FIG. 12C) after 30 seconds of incubation. After 10 minutes of incubation with His₆-hPGES, about half of the 15-Hydroperoxy PGE₂ was converted to PGE₂ (FIG. 12D), whereas the buffer control showed a typical non-enzymatic profile with 3:1 ratio of PGE₂:PGD₂ (FIG. 12E) (Nugteren et al. (1980) dv. In Prostaglandin and Thromboxane Res. 6, edited by B. Samelsson, P. W. Ramwell, and R. Paoletti. Raven Press: NY, 129-137). When His₆-hPGES was incubated with PGG₂ in the absence of reduced glutathione, no reduction of 15-Hydroperoxy PGE₂ took place (FIG. 12F). Boiled enzyme did not contain any activity either. His₆-hPGES was also incubated with 15-HP PGE₂ (as described for PGG₂ with 0.1 mg/ml purified his₆-hPGES and 0.16 mM 15 hydroperoxy-PGE₂ at 37° C.) but no peroxidase activity towards this compound was detected.

K_{M} and kcat for PGG₂ are presented in Table 1 and the specific activity at Vₘₐₓ in Table 2. Furthermore, his₆-PGES catalyzed the reduction of 15-hydroperoxy-PGE₂, although at a very low rate compared to the high-efficient isomerization of PGG₂ to 15-hydroperoxy-PGE₂. Due to the hydrophobic properties of PGG₂ and PGH₂, the K_{M} values were given as apparent.

Thus, purified His₆-hPGES could catalyze the conversion of PGG₂ into 15-HP PGE₂ and 15-HP PGE₂ was then reduced non-enzymatically in a slower step to PGE₂ in the presence of GSH. A person of skill in the art would understand that 15-HP PGE₂ could be an important and extremely short-lived intermediate with yet unknown biological functions. Due to the instability of PGG₂ in aqueous solution and an inadequate stop-solution, an acceptable Michaelis-Menten saturation curve with PGG₂ could not be obtained. However, the highest measured specific activity is presented in Table 2.

TABLE 1
Steady state kinetic parameters for PGES obtained at 37° C.

| Substrate | K_{M} (mM) | kcat (s⁻¹) | kcat/KM (M⁻¹ s⁻¹) |
|---|---|---|---|
| PGG₂ | 0.160 ± 0.03 | 75 ± 4 | 470 × 10³ |
| PGH₂ | 0.160 ± 0.04 | 50 ± 6 | 310 × 10³ |
| GSH | 0.71 ± 0.2 | 21 ± 1 | 30 × 10³ |

TABLE 2
Specific activities determined at Vₘₐₓ with the purified His₆-hPGES
and different substrates

| Substrate | Temp. (° C.) | Specific activity (μmol/min mg) |
|---|---|---|
| PGH₂ | 0 | 15 |
| PGH₂ | 37 | 170 ± 20 |
| LTA₄ | 37 | Not detectable above background |
| CDNB | 30 | 0.3 ± 0.1 |
| 5-HPETE | 37 | 0.043 ± 0.001 |
| PGG₂ | 37 | 250 ± 15 |
| 15-hydroperoxy- | 37 | 0.043 ± 0.019 |
| PGE₂ | | |
| Cumene hydro- | 37 | 0.17 ± 0.02 |
| peroxide | | |

Thus, from the substrates so far tested, a person of skill of art would understand that, hPGES has a high specific activity for PGH₂, and that PGES is also capable of catalyzing the conversion of CDNB; 5-HPETE and PGG₂.

For purposes of comparison, Table 3 lists characteristics of enzymes with PGE synthase activity.

TABLE 3
Kinetic properties of enzymes with PGE synthase activity

| | | MW | Spec. Act. | | | |
|---|---|---|---|---|---|---|
| Enzyme | Source | (kDa) | (μmol.min⁻¹.mg⁻¹) | Km (μM) | k_{cat} (s⁻¹) | k_{cat}/Km |
| GSH - | Bovine, heart¹ | 31 | 0.83 | 24 | 0.43 | 18 |
| independent | | | | | | |
| PGES | | | | | | |
| Cytosolic PGES, | Human, brain² | 24.5 | 0.38 | 147 | 0.27 | 1.8 |
| anionic GST | | | | | | |
| Cytosolic PGES, | Human, brain³ | 26.5 | 0.28 | 141 | 0.18 | 1.28 |
| GST M2-2 | | | | | | |
| Cytosolic PGES, | Human, brain³ | 27.5 | 0.92 | 1500 | 2.17 | 1.44 |
| GST M3-3 | | | | | | |
| Cytosolic PGES | Rat, brain⁴ | 26 | 5 | 15 | 2 | 140 |
| LPS-induced | | | | | | |
| hPGES | Human, | 18 | 167 | 163 | 50 | 310 |
| (MGST1-L1) | recombinant | | | | | |

¹Watanabe et al, BBA 1999
²Ogorochi et al, (1987) J. Neurochem.48:900;
³Beuckmann et al. (2000) Neurochem Res. 25:733;
⁴Tanioka et al. (2000) J. Biol. Chem. 275:32775.

### Example 11

### Electron Crystallography

Purified his₆-hPGES in 1% Triton X-100 was subjected to 2-D crystallization trials by adding phospholipids prior to reduction of the detergent content (Hebert (1998) Biomembrane Structures (Chapman, D., and Haris, P., eds), pp 88-110, IOS Press and Mosser (2001) Micron 32: 517). Specimens showing crystalinity, as judged from analysis of negatively stained samples by transmission electron microscopy and optical diffraction, were subjected to preparation by the back injection method using a 1% tannin solution as stabilizing agent. The specimens were frozen at −175° C. and kept at approximately this temperature in a Gatan 626 cryo holder throughout data collection. (Wang et al. (1991) J. Mol. Biol., 217:691 and Hirai et al. (1999) J. Electron Microsc. 48:653). Electron microgrpahs were recorded on Kodak SO-163 film using a philips CM120 electron microscope operated at 120 kV. Selected areas were digitized in a Zeiss Scai scanner at 7 μm pixel size corresponding to 1.4 Å on the specimen level. The data was subjected to several steps of image processing essentially as described using programs from the MRC suite (Henderson et al. (1986) Ultramicroscopy 19:147 and Cowther et al. (1996) J. Struct. Biol. 116:9).

Two-dimensional crystals of his₆-hPGES had unit cell parameters, a=97.0, b=98.0 Å, γ=90.0°. The present data is consistent with assigning projection symmetry pgg with an overall phase residual of 25.7° between symmetry related diffraction spots to a resolution of 10 Å (Table 4). The glide lines parallel the a- and b-axes of the crystal give rise to horizontal and vertical rows of symmetry related protein units in alternating up and down orientations as depicted in FIG. 12. The projection map was obtained by merging data from 18 crystalline areas.

TABLE 4
Phase residuals from the merged electron crystallographic data set of
his6-hPGES 2-D crystals

| | Phase | Number | Phase deviation | |
|---|---|---|---|---|
| Resolution | deviation from | of | between symmetry | Number of |
| range (Å) | 0° or 180° | spots | related spots* | comparisons |
| 100-20 | 15.2 | 30 | 4.4 | 13 |
| 20-15 | 19.6 | 32 | 11.8 | 15 |
| 15-12 | 27.5 | 36 | 29.9 | 17 |
| 12-10 | 36.9 | 38 | 47.3 | 19 |
| 100-10 | 25.6 | 136 | 25.7 | 64 |

*Phase relationships in projection symmetry pgg: α(h,k) = (h,−k) for h + k = 2n and α(h,k) = α(h,−k) + 180 for h + k = 2n + 1.

The molecular mass of the his₆-hPGES protein in relation to the unit cell isze suggests that each of the protein units in the 2-D map (encircled in FIG. 13) corresponds to the projection of three monomers along the direction perpendicular to the plane of the membrane. The resulting packing density is 22.7 D/Å², which is comparable to 2-D crystals of many other small integral membrane proteins. Thus, PGES constitutes a trimer in the membrane.

### Example 12

### Molecular Weight of the PGES-Triton X-100 Complex

The degree of Triton X-100 binding to the purified his6-hPGES was determined in a series of experiments. The sedimentation coefficient, partial specific volume and stokes radius were determined by the use of sucrose gradients and gel filtration on sephacryl S-300 HR. The molecular weight of a protein-detergent complex can be calculated from the Svedberg equation

$$M = \frac{6 π {η}_{20 , w} {S}_{20 , w} aN}{1 - υ {Q}_{20 , w}}$$

M is the molar mass, η_{20,w} is the viscosity of water at 20° C., S_{20,w} is the sedimentation coefficient, a is the stokes radius, N is Avogadro's number, ν is the partial specific volume and Q_{20,w} is the density of water at 20° C.

The sedimentation coefficient of the PGES-Triton X-100 complex was determined by adding 100 μg of purified his₆-hPGES together with 0.5 mg cytochrome c and 10 mg bovein serum albumin in a totla volume of 450 μl on top of a 10 ml linear gradient containing 5-20% sucrose, 10 mM potassium phosphate buffer pH 7.4, 1 mM GSH, 1% Triton X-100. Cytochrome c and bovine serum albumin were used as standards with sedimentaion coefficients of 1.7S and 4.6S, respectively. Centrifugation was performed as 160 000×gav in a Beckman SW40Ti rotor for 45 hours at 4° C. Fractions were then collected from the bottom of the tubes with a syringe using a pump and 0.4 ml aliquots were saved for activity assays and proteni determination. These fractions were assayed for PGES activity as described herein and the refractive index was determined. Cytochrome c was determined from the absorbance at 405 m and bovine serum albumin localized by measuring protein (Pande et al. (1994) Anal. Biochem. 220:424).

The partial specific volume of the PGES-Triton X-100 compelx was determined by equilibrium density gradient centrifugation. 30 μg of purified his₆-hPGES was added to a 3.8 ml gradient containing 30-60% sucrose, 10 mM potassium phosphate buffer pH 7.4, 1 mM GSH, 1% Triton X-100. The tubes were centrifuged at 246 000 gₐᵥ in a Beckman SW60Ti rotor for 72 hours and 96 hours at 4° C. to ensure that equilibrium had been reached. Fractions of 0.16 ml wre collected and analyzed as described above.

The Stokes radius of the PGES-Triton X-100 complex was determined by gel exclusion chromatography on a column (1.6×60 cm) of Sephacryl S-300 HR. 30-50 μg of purified his₆-hPGES was loaded on the column together with marker enzymes in 1.0 ml elution buffer. Elution was performed with 50 mM soidum phosphate buffer pH 7.4, 150 mM NaCl, 1 mM GSH, 0.2% Triton X-100 and 0.5% glycerol at 10° C. Fractions of 1.0 m. were collected and analyzed as described above. A HMW gel filtration calibration kit from Amersham Pharmacia Biotech was used. The marker enzymes used were albumin, catalase, ferritin and thyroglobulin. Neither of the markers has been shown to bind significant amounts of detergents. The Stokes radius was estimated according to the Gel filtration kit instruction manual from Amersham Pharmacia Biotech.

The sedimenation coefficient was of the PGES-Triton X-100 complex was found to be 3.4±0.2 S (n=2) (FIG. 14A) and the partial specific volume was 0.879±0.006 cm³/g 9n=5), which correspons to a density of 1.14 g/ml (33% sucrose) as seen in FIG. 10B. The PGES-Triton X-100 complex almost co-cromatographed with catalase on the sephacryl S-300 HR column. The square root of −log K_{AV} values were plotted against the known Stokes radii of the marker enzymes and the Stokes radius of the PGES-Triton X-100 complex was shown to be 5.2±0.1 nm (n=2). The gel filtration was repeated with 0.5% reduced Triton X-100 and no significant differences in Stokes radius of the PGES-Triton X-100 complex was found (FIG. 14C). Gel filtration was also performed with marker enzymes alone and they did not seem to be affected by the Triton X-100 in the elution buffer.

These data were the substituted into the Svedberg equation and the molecular weight of the PGES-Triton X-100 complex was found to be 164 000. To determine how much Triton X-100 this complex consists of, it is necessary to know the partial specific volume of both PGES and Triton X-100. Triton X-100 has a partial specific volume of 0.939 cm³/g and the partial specific volumen of PGES is calculated from its known amino acid sequence (0.75 cm³/g). Triton has a mean molecular weight of 625 and subsequent calculations gave a value of 2.15 g Triton X-100 bound/g PGES, which corresponds to three polypeptide chains and 185 detergent molecules per complex.

### Example 13

### Induction of PGE Synthase and Cyclooxygenase-2 in A549 Cells

This Example describes the induction of active PGES polypeptide by IL-1β to A549 cells and its comparison with that of COX-2.

Microsomal fractions of A549 cells cultured with or without IL-1 β 1 ng/ml) for 0, 12, 24, 36 or 72 hours were subjected to SDS-PAGE and Western blot analysis. As positive control, the membrane fraction from bacteria expressing human PGE synthase was included. In all lanes of the gel, 10 μg of total protein was analyzed, except for the human PGE synthase where 2 μg was loaded. The Western blot was then incubated with antipeptide antiserum against PGE synthase. The blot was then stripped and incubated with antiserum against COX-2.

FIG. 15A demonstrates the induction of the microsomal PGE synthase protein as a function of time after addition of IL-1β to A549 cells. FIG. 15B demonstrates the corresponding expression profile of cyclooxygenase-2.

In order to study the possible correlation between the result from the Western blot and PGE synthase activity, the corresponding microsomes were incubated in the presence of PGH₂ and reduced glutathione as described above. Briefly, microsomes (0.03 mg/ml) were incubated with PGH₂ (11 μM) for 30 seconds at 0° C., followed by extraction and analysis by RP-HPLC. In FIG. 16, the PGE synthase activity in microsomes from A549 cells treated with IL-1β was shown to increase over time in contrast to non-treated cells (controls). In addition, no corresponding activity was detected in the cytosolic fractions.

Thus, a person of skill in the art would recognize that the results illustrate a tandem upregulation of COX-2 and PGE synthase proteins and that the microsomal PGE synthase activity correlated to the induction of PGE synthase protein.

Equivalents

Those skilled in the art will recognize, or be able to ascertain using no more than routine experimentation, many equivalents of the specific embodiments of the invention described herein. Such equivalents are intended to be encompassed by the following claims.

1. A substantially pure preparation of Prostaglandin E Synthase (PGES) comprising SEQ ID NO 2, or analog or variant thereof.

2. The preparation of claim 1, wherein the PGES or analog or variant thereof is fused to a heterologous polypeptide, thereby forming a fusion polypeptide.

3. The preparation of claim 1 or 2, having less than about 10% contaminating cellular material.

4. The preparation of claim 3, having less than about 5% contaminating cellular material.

5. The preparation of claim 4, having less than about 1% contaminating cellular material.

6. The preparation of claim 5, having less than about 0.1% contaminating cellular material.

7. The preparation of claim 1 or 2, which is substantially free of other cellular proteins.

8. The preparation of claim 7, having less than about 10% contaminating proteins.

9. The preparation of claim 8, having less than about 5% contaminating proteins.

10. The preparation of claim 9, having less than about 1% contaminating proteins.

11. The preparation of claim 10, having less than about 0.1% contaminating proteins.

12. The preparation of claim 1, wherein the PGES polypeptide, or analog or variant thereof, is capable of catalyzing the conversion of a PGH into a PGE.

13. The preparation of claim 12, wherein the PGES polypeptide, or analog or variant thereof, has a specific activity of at least about 100 μmol/min/mg, when the activity is measured at 37° C.

14. The preparation of claim 12, wherein the PGES polypeptide, or analog or variant thereof has a specific activity of at least about 10 μmol/min/mg, when the activity is measured at 0° C.

15. The preparation of claim 2, wherein the fusion polypeptide is capable of catalyzing the conversion of a PGH into a PGE.

16. The preparation of claim 15, wherein the fusion polypeptide has a specific activity of at least about 100 μmol/min/mg, when the activity is measured at 37° C.

17. The preparation of claim 15, wherein the fusion polypeptide has a specific activity of at least about 10 μmol/min/mg, when the activity is measured at 0° C.

18. The preparation of claim 2, wherein the heterologous polypeptide mediates binding of the fusion polypeptide to an affinity matrix.

19. The preparation of claim 18, wherein the affinity matrix is a metal ion affinity matrix.

20. The preparation of claim 19, wherein the metal ion affinity matrix is a Ni²⁺ chelate affinity matrix.

21. The preparation of claim 20, wherein the heterologous polypeptide comprises from one to twelve consecutive histidines.

22. The preparation of claim 21, wherein the fusion polypeptide comprises SEQ ID NO: 4.

23. The preparation of claims 1 or 2, which comprises about 10% glycerol.

24. The preparation of claims 1 or 2, which comprises about 1-5 mM reduced glutathione.

25. The preparation of claims 1 or 2, which comprises about 10% glycerol and about 1-5 mM reduced glutathione.

26. The preparation of claim 25, which is frozen.

27. A method for obtaining a substantially pure preparation of a PGES fusion polypeptide, comprising
(i) providing a lysate comprising a PGES fusion polypeptide comprising a PGES polypeptide, analog or fragment thereof fused to a heterologous polypeptide capable of binding to a metal ion affinity chromatography;
(ii) subjecting the lysate of (i) to hydroxyapatite (HA) chromatography;
(iii) obtaining the unbound fraction from the HA chromatography;
(iv) subjecting the unbound fraction from the HA chromatography to metal ion affinity chromatography, in conditions appropriate for the fusion polypetide to bind to the metal ion; and
(v) eluting the metal ion affiniyt chromatography, to thereby obtain a substantially pure preparation of a PGES fusion polypeptide.

28. The method of claim 27, wherein the lysate is a whole cell lysate.

29. The method of claim 27, wherein the lysate is a solubilized membrane fraction of a cell.

30. The method of claim 28, wherein the lysate is from a prokaryotic cell.

31. The method of claim 30, wherein the prokaryote is an E. coli bacteria.

32. The method of claim 27, wherein the HA chromatography is performed in the presence of about 10% glycerol and/or about 1-5 mM reduced glutathione.

33. The method of claim 27, wherein the metal ion chromatography is performed in the presence of about 10% glycerol and/or about 1-5 mM reduced glutathione.

34. The method of claim 27, wherein each step of the method is performed in solutions comprising about 10% glycerol and/or about 1-5 mM reduced glutathione.

35. The method of claim 34, wherein metal ion is Ni²⁺.

36. The method of claim 35, wherein the heterologous polypeptide comprises at least six consecutive histidines.

37. The method of claim 27, wherein the fusion polypeptide further comprises a protease recognition site between the PGES polypeptide or analog or fragment thereof and the heterologous polypeptide, and the method further comprises subjecting the fusion polypeptide to the protease recognizing the protease recognition site after the metal ion affinity chromatography, such that the heterologous polypeptide is cleaved from the PGES polypeptide or analog or fragment thereof.

38. A purified PGES fusion polypeptide prepared by the method of claim 27.

39. A purified PGES polypeptide or analog or fragment thereof, prepared by the method of claim 37.

40. A pharmaceutical composition comprising the preparation of claim 1.

41. A method for identifying an agent which modulates the interaction between a PGES polypeptide or analog or fragment thereof and a PGES-binding partner, comprising contacting a substantially pure PGES polypeptide or analog or fragment thereof; a PGES-binding partner; and a test agent, in conditions under which, but for the presence of the test agent, the PGES polypeptide or analog or fragment thereof and the PGES binding partner interact, wherein a difference in the interaction between the PGES polypeptide or analog or fragment thereof and the PGES-binding partner indicates that the test agent modulates their interaction.

42. The method of claim 41, wherein the PGES polypeptide or analog or fragment thereof is fused to a heterologous polypeptide which binds a metal ion affinity chromatography to form a fusion polypeptide.

43. The method of claim 42, wherein the fusion polypeptide is prepared according to the method of claim 27.

44. A method for in vitro preparing PGE₂, comprising contacting PGH₂ and a substantially pure preparation of PGES or analog of fragment thereof of claim 1 or fusion polypeptide of claim 2 and reduced glutathione, under conditions appropriate for the catalysis of the conversion of a PGH into a PGE by PGES.

45. A method for treating a subject in need of modulation of PGES or prostaglandin activities, comprising administering to the subject a pharmaceutically efficient amount of a modulator of PGES, such that the subject is treated.

46. A method for treating a subject in need of prostaglandin, comprising administering to the subject a pharmaceutically efficient amount of prostaglandin prepared by the method of claim 43, such that the subject is treated.

47. A method for treating a subject in need of inhibiting prostaglandin production, comprising administering to the subject a preparation of claim 1 or 2, wherein the PGES polypeptide or analog or fragment thereof is a dominant negative mutant of naturally-occurring PGES.

48. A method for treating a subject in need of inhibiting prostaglandin production, comprising administering to the subject a compound identified by the method of claim 41.

49. A method for determining the three dimensional structure of a PGES polypeptide or analog or fragment thereof, comprising crystalizing the preparation of claim 1; determining the coordinates of the crystal structure; and introducing the coordinates into a computer program allowing the determination of three dimensional structures from coordinates.

50. A method for identifying drugs that modulate the catalytic activity of a naturally-occurring PGES polypeptide, comprising determining the three dimensional structure of PGES polypeptide according to the method of claim 47 and selecting drugs that interact with the three dimensional structure.

51. An isolated nucleic acid encoding a human PGES polypeptide comprising the amino acid sequence set forth in SEQ ID NO: 2 or which differs from the sequence in 1 to about 20 amino acids, which PGES polypeptide is fused to a His₆ tag.

52. The isolated nucleic acid of claim 51, wherein the PGES polypeptide consists of SEQ ID NO: 2.

53. A vector comprising the isolated nucleic acid of claim 51.

54. The vector of claim 53, having a nucleotide sequence which is at least about 95% identical to the sequence set forth in FIG. 2.

55. An isolated cell comprising the nucleic acid of claim 51.


SEQUENCE LISTING (6 sequences)

SEQ ID NO: 1 - DNA, 459 bp, Homo sapiens
  CDS (1)..(459)
atgcctgcccacagcctggtgatgagcagcccggccctcccggccttcctgctctgcagcacgctgctggtcatcaagatgtacgtggtggccatcatcacgggccaagtgaggctgcggaagaaggcctttgccaaccccgaggatgccctgagacacggaggcccccagtattgcaggagtgaccccgacgtggaacgctgcctcagggcccaccggaacgacatggagaccatctaccccttccttttcctgggcttcgtctactcctttctgggtcctaacccttttgtcgcctggatgcacttcctggtcttcctcgtgggccgtgtggcacacaccgtggcctacctggggaagctgcgggcacccatccgctccgtgacctacaccctggcccagctcccctgcgcctccatggctctgcagatcctctgggaagcggcccgccacctgtga

SEQ ID NO: 2 - protein, 152 aa, Homo sapiens
MPAHSLVMSSPALPAFLLCSTLLVIKMYVVAIITGQVRLRKKAFANPEDALRHGGPQYCRSDPDVERCLRAHRNDMETIYPFLFLGFVYSFLGPNPFVAWMHFLVFLVGRVAHTVAYLGKLRAPIRSVTYTLAQLPCASMALQILWEAARHL

SEQ ID NO: 3 - DNA, 480 bp, Homo sapiens
  CDS (1)..(480)
atgcatcaccatcaccatcatatgcctgcccacagcctggtgatgagcagcccggccctcccggccttcctgctctgcagcacgctgctggtcatcaagatgtacgtggtggccatcatcacgggccaagtgaggctgcggaagaaggcctttgccaaccccgaggatgccctgagacacggaggcccccagtattgcaggagtgaccccgacgtggaacgctgcctcagggcccaccggaacgacatggagaccatctaccccttccttttcctgggcttcgtctactcctttctgggtcctaacccttttgtcgcctggatgcacttcctggtcttcctcgtgggccgtgtggcacacaccgtggcctacctggggaagctgcgggcacccatccgctccgtgacctacaccctggcccagctcccctgcgcctccatggctctgcagatcctctgggaagcggcccgccacctgtga

SEQ ID NO: 4 - protein, 159 aa, Homo sapiens
MHHHHHHMPAHSLVMSSPALPAFLLCSTLLVIKMYVVAIITGQVRLRKKAFANPEDALRHGGPQYCRSDPDVERCLRAHRNDMETIYPFLFLGFVYSFLGPNPFVAWMHFLVFLVGRVAHTVAYLGKLRAPIRSVTYTLAQLPCASMALQILWEAARHL

SEQ ID NO: 5 - DNA, 4956 bp, Homo sapiens
actcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagagcttgcaattcgcgcgcgaaggcgaagcggcatttacgttgacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgggtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctaatacgactcactatagggaaagctggtacgcctgcaggtaccggtccggaattcccgggtcgacgagctcactagtcggcggccgctctagaaataattttgtttaactttaagaaggagatatacaaatgcatcaccatcaccatcatatgcctgcccacagcctggtgatgagcagcccggccctcccggccttcctgctctgcagcacgctgctggtcatcaagatgtacgtggtggccatcatcacgggccaagtgaggctgcggaagaaggcctttgccaaccccgaggatgccctgagacacggaggcccccagtattgcaggagcgaccccgacgtggaacgctgcctcagggcccaccggaacgacatggagaccatctaccccttccttttcctgggcttcgtctactcctttctgggtcctaacccttttgtcgcctggatgcacttcctggtcttcctcgtgggccgtgtggcacacaccgtggcctacctggggaagctgcgggcacccatccgctccgtgacctacaccctggcccagctcccctgcgcctccatggctctgcagatcctctgggaagcggcccgccacctgtgaagcttgagtattctatagtgtcacctaaatcccagcttgatccggctgctaacaaagcccgaaaggaagctgagttggctgctgccaccgctgagcaataactagcataaccccttggggcctctaaacgggtcttgaggggttttttgctgaaaggaggaactatatccggataacctggcgtaatagcgaagaggcccgcaccgatcgcccttcccaacagttgcgcagcctgaatggcgaatggaaattgtaaacgttaatattttgttaaaattcgcgttaaatatttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcacccaaatcaagttttttgcggtcgaggtgccgtaaagctctaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccaaacccgccgcgcttaatgcgccgctacagggcgcgtcgcctgatgcggtattttctccttacgcatctgtgcggtatttcacaccgcatatatggtgcactctcagtacaatctgctctgatgccacatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtcatacactattatcccgtattgacgccgggcaagagcaactcggtcgccgggcgcggtattctcagaatgaattggttgagt

SEQ ID NO: 6 - protein, 6 aa, Artificial Sequence
  Description of Artificial Sequence Synthetic 6X-His tag
HHHHHH